(19) United States
(12) Patent Application Publication
(10) Pub. No.: US 2010/0022514 A1
(43) Pub. Date: Jan. 28, 2010

(54) Heterocycle Compounds and Methods of Use Thereof

(75) Inventors: Young Shin Cho, Cambridge, MA (US); Lei Jiang, Cambridge, MA (US); Michael Shultz, Cambridge, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: NORVARTIS AG

(21) Appl. No.: 12/519,319

(22) PCT Filed: Dec. 17, 2007
(86) PCT No.: PCT/US07/87707

§ 371 Date: Jun. 15, 2009

Related U.S. Application Data
(60) Provisional application No. 60/870,176, filed on Dec. 15, 2006.

(51) Int. Cl. A61K 31/4035; C07D 209/44; C07D 403/06; C07D 401/12; C07D 471/04; C07D 217/04; A61K 31/55; A61K 31/4439; A61K 31/4178; A61K 31/437; A61K 31/472; A61K 31/4725; A61P 35/00; A61P 37/00; A61P 25/00

(52) U.S. Cl. 514/213.01; 548/482; 548/455; 540/594; 540/593; 546/277.1; 548/312.1; 546/121; 546/146; 514/416; 514/414; 514/217.01; 514/339; 514/397; 514/300; 514/307

(57) ABSTRACT

The invention relates to the use of compounds in the treatment of deacetylase-associated diseases and for the manufacture of pharmaceutical preparations for the treatment of said diseases.

### BACKGROUND

The present application claims priority to U.S. Ser. No. 60/870,176, filed Dec. 15, 2006, herein incorporated by reference. In all eukaryotic cells, genomic DNA in chromatin associates with histones to form nucleosomes. Each nucleosome consists of a protein octamer made up of two copies of each histone: H2A, H2B, H3 and H4. DNA winds around this protein core, with the basic amino acids of the histones interacting with the negatively charged phosphate groups of the DNA. The most common post translational modification of these core histones is the reversible acetylation of the ε-amino groups of conserved highly basic N-terminal lysine residues. The steady state of histone acetylation is established by the dynamic equilibrium between competing histone acetyltransferase(s) and histone deacetylase(s) herein referred to as HDAC.

The dynamic equilibrium between histone acetylation and deacetylation is essential for normal cell growth. Inhibition of histone deacetylation results in cell cycle arrest, cellular differentiation, apoptosis and reversal of the transformed phenotype. Therefore, HDAC inhibitors can have great therapeutic potential in the treatment of cell proliferative diseases or conditions. For example, butyric acid and its derivatives, including sodium phenylbutyrate, have been reported to induce apoptosis in vitro in human colon carcinoma, leukemia and retinoblastoma cell lines. However, butyric acid and its derivatives are not useful pharmacological agents because they tend to be metabolized rapidly and have a very short half-life in vivo. Other inhibitors of HDAC that have been widely studied for their anti-proliferative activities are trichostatin A and trapoxin. Trichostatin A is an antifungal and antibiotic and is a reversible inhibitor of mammalian HDAC. Trapoxin is a cyclic tetrapeptide, which is an irreversible inhibitor of mammalian HDAC. Although trichostatin and trapoxin have been studied for their anti-cancer activities, their in vivo instability makes them less suitable as anti-cancer drugs.

Other deacetylases act upon other proteins, including, e.g., p53, Hif-1α, tubulin, HSP90. Therefore, agonists and antagonists of such deacetylases would be useful in modulating acetylation on these and a diverse range of proteins that also are associated with diseases and disorders.

In view of the above, there is an ongoing need for inhibitors and/or antagonists of HDAC and other deacetylases.

### SUMMARY OF THE INVENTION

The present invention provides deacetylase-inhibitor compounds. In particular, the present invention provides compounds of formula I. In a particular embodiment, the present invention provides isoindoline derivatives. In another embodiment, the present invention provides tetrahydro-isoquinoline derivatives. In another embodiment, the present invention provides tetrahydro-benzazepine derivatives. The compounds of the invention, also referred to herein as deacetylase-inhibitor compounds, are suitable for treating deacetylase-associated disorders, e.g., for the treatment of a proliferative disease, a hyperproliferative disease, a disease of the immune system, a disease of the central or peripheral nervous system, or a disease associated with misexpression of a gene. The present invention includes heterocyclic compounds that are efficacious deacetylase inhibitors.

In one aspect, the invention provides a method of treating a deacetylase-associated disorder. The method includes administering to a subject in need thereof a pharmaceutically acceptable amount of a heterocyclic compound such that the deacetylase-associated disorder is treated. In one embodiment, the heterocyclic compound is of the Formula I:

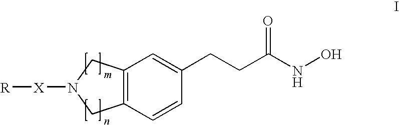

In yet another aspect, the invention provides a method of treating a proliferative disease by administering to a subject in need thereof a pharmaceutically acceptable amount of a compound of the Formula I, such that the proliferative disease is treated.

In still another aspect, the invention provides a packaged deacetylase-associated disorder treatment. The treatment includes a deacetylase-modulating compound of the Formula I, packaged with instructions for using an effective amount of the deacetylase-modulating compound to treat a deacetylase-associated disorder.

### DETAILED DESCRIPTION OF THE INVENTION

The present invention provides deacetylase-inhibitor compounds. In a particular embodiment, the present invention provides isoindoline compounds. In another embodiment, the present invention provides tetrahydro-isoquinoline compounds. In a particular embodiment, the invention provides tetrahydro-benzazepine compounds. A function of these compounds includes, for example, inhibition of deacetylases or inhibition of histone Deacetylases, and the use of such modulators for treatment of deacetylase-associated disorders, e.g., for the treatment of a proliferative disease, a hyperproliferative disease, a disease of the immune system, a disease of the central or peripheral nervous system, or a disease associated with misexpression of a gene.

In one aspect, the compounds of the invention are of the Formula I:

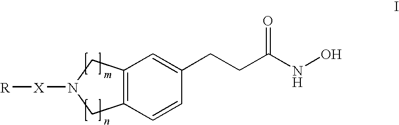

wherein

the dashed line indicates a single or double bond,

n and m are each, independently, 1, 2, or 3, and the sum of n and m is 2, 3 or 4;

wherein X is (CH₂)ⱼ wherein each CH₂ may be independently replaced one or more times with C(O), S(O)₂, S(O), O, or NR², wherein R² is selected from the group consisting of H, alkyl, aryl, heterocycle, C₁₋₄-alkyl, and C₃₋₆-cycloalkyl;

j is an integer between 0 and 6.

R is selected from the group consisting of C₁₋₄-alkyl, C₃₋₆-cycloalkyl and aryl, wherein cycloalkyl and aryl may be further independently substituted one or more times with aryl, heterocycle, C₁₋₄-alkyl, C₁₋₄-alkoxy, halogen, amino, nitro, cyano, pyrrolidinyl or CF₃.

Preferred embodiments of the compounds of the invention, also referred to herein as deacetylase-inhibitor compounds, (including pharmaceutically acceptable salts thereof) are shown below in Table A, and are also considered to be “compounds of the invention.”

TABLE A

| | | | HDAC-1 | p21 | LCMS |
|---|---|---|---|---|---|
| Example | Structure | IUPAC Name | IC₅₀ | AC₅₀ | (M + 1) |
| 1 | 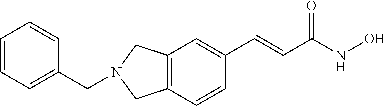 | (E)-3-(2-Benzyl-2,3-dihydro-1H- isoindol-5-yl)-N-hydroxy- acrylamide | ** | * | 295.02 |
| 2 | 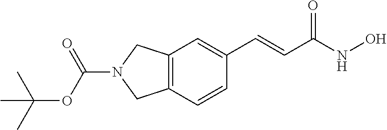 | 5-((E)-2-Hydroxycarbamoyl-vinyl)- 1,3-dihydro-isoindole-2-carboxylic acid tert-butyl ester | ** | * | 305.06 |
| 3 | 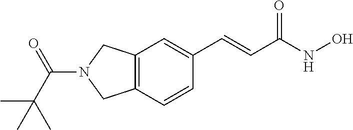 | (E)-3-[2-(2,2-Dimethyl-propionyl)- 2,3-dihydro-1H-isoindol-5-yl]-N- hydroxy-acrylamide | ** | * | 289.1 |
| 4 | 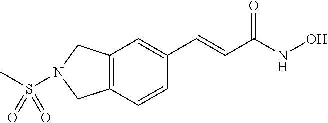 | (E)-N-Hydroxy-3-(2- methanesulfonyl-2,3-dihydro-1H- isoindol-5-yl)-acryl amide | ** | * | 283.0 |
| 5 | 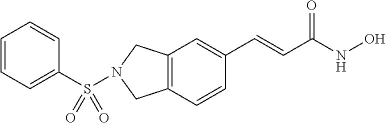 | (E)-3-(2-Benzenesulfonyl-2,3- dihydro-1H-isoindol-5-yl)-N- hydroxy-acrylamide | ** | * | 345.0 |
| 6 | 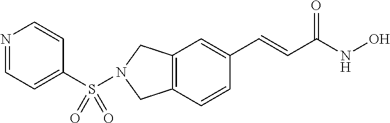 | (E)-N-Hydroxy-3-[2-(pyridine-4- sulfonyl)-2,3-dihydro-1H-isoindol- 5-yl]-acrylamide | ** | * | 310.0 |
| 7 | 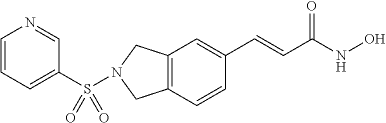 | (E)-N-Hydroxy-3-[2-(pyridine-3- sulfonyl)-2,3-dihydro-1H-isoindol- 5-yl]-acrylamide | ** | * | 346.0 |
| 8 | 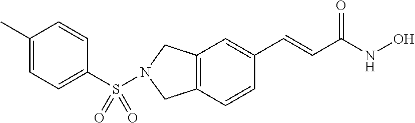 | (E)-N-Hydroxy-3-[2-(toluene-4- sulfonyl)-2,3-dihydro-1H-isoindol- 5-yl]-acrylamide | *** | * | 359.01 |
| 9 | 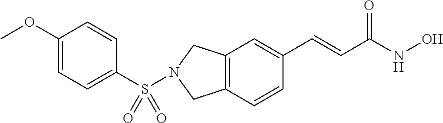 | (E)-N-Hydroxy-3-[2-(4-methoxy- benzenesulfonyl)-2,3-dihydro-1H- isoindol-5-yl]-acrylamide | *** | * | 375.0 |
| 10 | 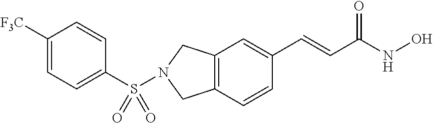 | (E)-N-Hydroxy-3-[2-(4- trifluoromethyl-benzenesulfonyl)- 2,3-dihydro-1H-isoindol-5-yl]- acrylamide | ** | * | 413.1 |
| 11 | 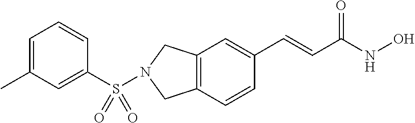 | (E)-N-Hydroxy-3-[2-(toluene-3- sulfonyl)-2,3-dihydro-1H-isoindol- 5-yl]-acrylamide | *** | * | 359.01 |
| 12 | 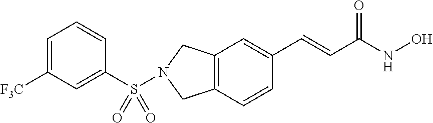 | (E)-N-Hydroxy-3-[2-(3- trifluoromethyl-benzenesulfonyl)- 2,3-dihydro-1H-isoindol-5-yl]- acrylamide | ** | * | 412.98 |
| 13 | 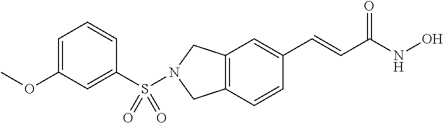 | (E)-N-Hydroxy-3-[2-(3-methoxy- benzenesulfonyl)-2,3-dihydro-1H- isoindol-5-yl]-acrylamide | *** | * | 375.0 |
| 14 | 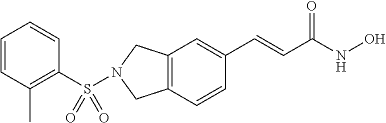 | (E)-N-Hydroxy-3-[2-(toluene-2- sulfonyl)-2,3-dihydro-1H-isoindol- 5-yl]-acrylamide | ** | * | 359.0 |
| 15 | 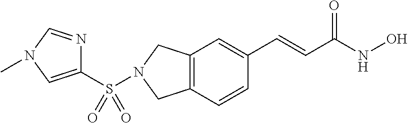 | (E)-N-Hydroxy-3-[2-(1-methyl-1H- imidazole-4-sulfonyl)-2,3-dihydro- 1H-isoindol-5-yl]-acrylamide | ** | * | 349.0 |
| 16 | 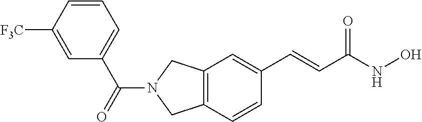 | (E)-N-Hydroxy-3-[2-(3- trifluoromethyl-benzoyl)-2,3- dihydro-1H-isoindol-5-yl]- acrylamide | ** | * | 377.0 |
| 17 | 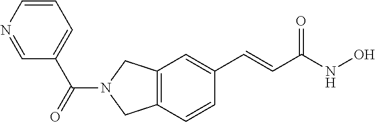 | (E)-N-Hydroxy-3-[2-(pyridine-3- carbonyl)-2,3-dihydro-1H-isoindol- 5-yl]-acrylamide | ** | * | 310.0 |
| 18 | 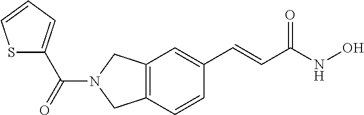 | (E)-N-Hydroxy-3-[2-(thiophene-2- carbonyl)-2,3-dihydro-1H-isoindol- 5-yl]-acrylamide | *** | * | 351.97 |
| 19 | 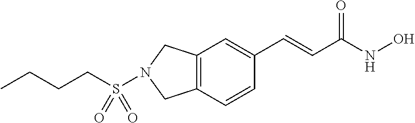 | (E)-3-[2-(Butane-1-sulfonyl)-2,3- dihydro-1H-isoindol-5-yl]-N- hydroxy-acrylamide | ** | * | 325.1 |
| 20 | 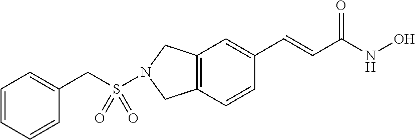 | (E)-N-Hydroxy-3-(2- phenylmethanesulfonyl-2,3- dihydro-1H-isoindol-5-yl)- acrylamide | ** | * | 359.1 |
| 21 | 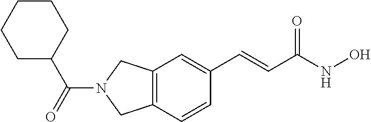 | (E)-3-(2-Cyclohexanecarbonyl-2,3- dihydro-1H-isoindol-5-yl)-N- hydroxy-acrylamide | ** | * | 313.1 (M − 1) |
| 22 | 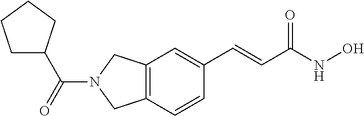 | (E)-3-(2-Cyclopentanecarbonyl-2,3- dihydro-1H-isoindol-5-yl)-N- hydroxy-acrylamide | ** | * | 301.1 |
| 23 | 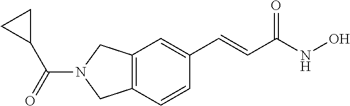 | (E)-3-(2-Cyclopropanecarbonyl- 2,3-dihydro-1H-isoindol-5-yl)-N- hydroxy-acrylamide | ** | * | 273.1 |
| 24 | 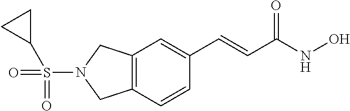 | (E)-3-(2-Cyclopropanesulfonyl-2,3- dihydro-1H-isoindol-5-yl)-N- hydroxy-acrylamide | ** | * | 309.1 |
| 25 | 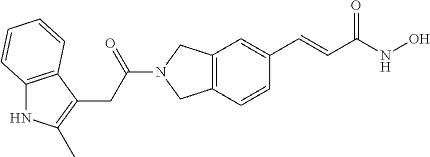 | (E)-N-Hydroxy-3-{2-[2-(2-methyl- 1H-indol-3-yl)-acetyl]-2,3-dihydro- 1H-isoindol-5-yl}-acrylamide | *** | * | 376.1 |
| 26 | 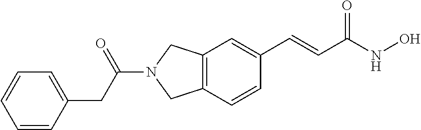 | (E)-N-Hydroxy-3-(2-phenylacetyl- 2,3-dihydro-1H-isoindol-5-yl)- acrylamide | ** | * | 323.2 |
| 27 | 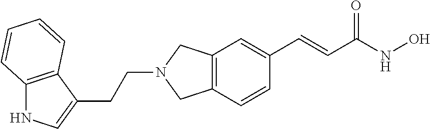 | (E)-N-Hydroxy-3-{2-[2-(1H-indol- 3-yl)-ethyl]-2,3-dihydro-1H- isoindol-5-yl}-acrylamide | *** | * | 348.1715 |
| 28 | 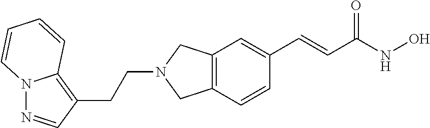 | (E)-N-Hydroxy-3-[2-(2- pyrazolo[1,5-a]pyridin-3-yl-ethyl)- 2,3-dihydro-1H-isoindol-5-yl]- acrylamide | *** | * | 349.1661 |
| 29 | 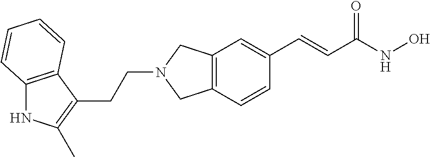 | (E)-N-Hydroxy-3-{2-[2-(2-methyl- 1H-indol-3-yl)-ethyl]-2,3-dihydro- 1H-isoindol-5-yl}-acrylamide | *** | ** | 362.1 |
| 30 | 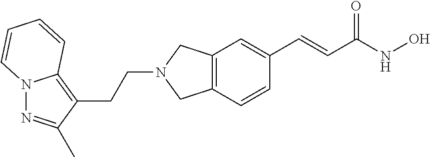 | (E)-N-Hydroxy-3-{2-[2-(2-methyl- pyrazolo[1,5-a]pyridin-3-yl)-ethyl]- 2,3-dihydro-1H-isoindol-5-yl}- acrylamide | | | 363.0 |
| 31 | 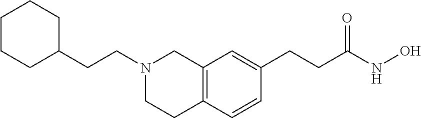 | (E)-3-[2-(2-Cyclohexyl-ethyl)- 1,2,3,4-tetrahydro-isoquinolin-7- yl]-N-hydroxy-acrylamide | ** | * | 331.1 |
| 32 | 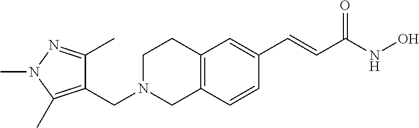 | (E)-N-Hydroxy-3-[2-(1,3,5- trimethyl-1H-pyrazol-4-ylmethyl)- 1,2,3,4-tetrahydro-isoquinolin-6- yl]-acrylamide | * | * | 341.2 |
| 33 | 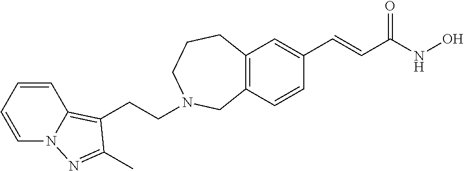 | (E)-N-Hydroxy-3-{2-[2-(2-methyl- pyrazolo[1,5-a]pyridin-3-yl)-ethyl]- 2,3,4,5-tetrahydro-1H- benzo[c]azepin-7-yl}-acrylamide | *** | * | 391.1 |
| 34 | 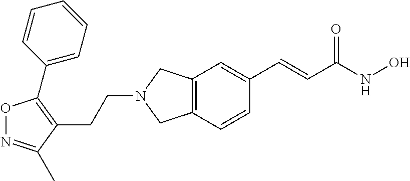 | (E)-N-Hydroxy-3-{2-[2-(3-methyl- 5-phenyl-isoxazol-4-yl)-ethyl]-2,3- dihydro-1H-isoindol-5-yl}- acrylamide | ** | * | 390.18 |
| 35 | 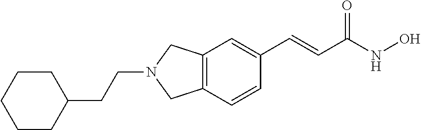 | (E)-3-[2-(2-Cyclohexyl-ethyl)-2,3- dihydro-1H-isoindol-5-yl]-N- hydroxy-acrylamide | ** | * | 315.1 |
| 36 | 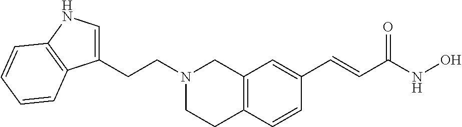 | (E)-N-Hydroxy-3-{2-[2-(1H-indol- 3-yl)-ethyl]-1,2,3,4-tetrahydro- isoquinolin-7-yl}-acrylamide | ** | * | 362.1866 |
| 37 | 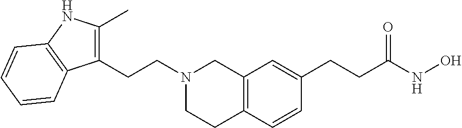 | N-Hydroxy-3-{2-[2-(2-methyl-1H- indol-3-yl)-ethyl]-1,2,3,4- tetrahydro-isoquinolin-7-yl}- propionamide | * | * | 392.0 |
| 38 | 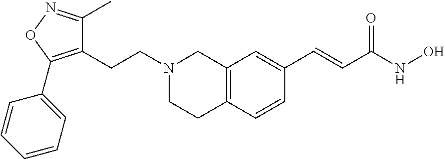 | (E)-N-Hydroxy-3-{2-[2-(3-methyl- 5-phenyl-isoxazol-4-yl)-ethyl]- 1,2,3,4-tetrahydro-isoquinolin-7- yl}-acrylamide | *** | * | 404.0 |
| 39 | 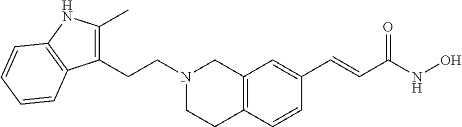 | (E)-N-Hydroxy-3-{2-[2-(2-methyl- 1H-indol-3-yl)-ethyl]-1,2,3,4- tetrahydro-isoquinolin-7-yl}- acrylamide | *** | ** | 376.0 |
| 40 | 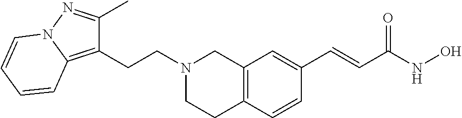 | (E)-N-Hydroxy-3-{2-[2-(2-methyl- pyrazolo[1,5-a]pyridin-3-yl)-ethyl]- 1,2,3,4-tetrahydro-isoquinolin-7- yl}-acrylamide | *** | * | 377.1 |
| 41 | 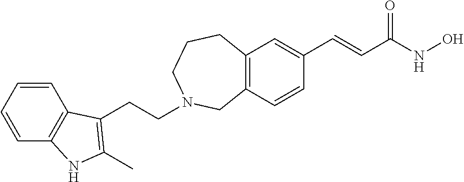 | (E)-N-Hydroxy-3-{2-[2-(2-methyl- 1H-indol-3-yl)-ethyl]-2,3,4,5- tetrahydro-1H-benzo[c]azepin-7- yl}-acrylamide | *** | * | 390.2182 |
| 42 | 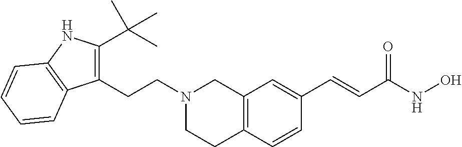 | (E)-3-{2-[2-(2-tert-Butyl-1H-indol- 3-yl)-ethyl]-1,2,3,4-tetrahydro- isoquinolin-7-yl}-N-hydroxy- acrylamide | *** | ** | 418.2480 |
| 43 | 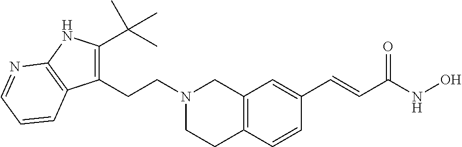 | (E)-3-{2-[2-(2-tert-Butyl-1H- pyrrolo[2,3-b]pyridin-3-yl)-ethyl]- 1,2,3,4-tetrahydro-isoquinolin-7- yl}-N-hydroxy-acrylamide | *** | * | 419.2437 |
| 44 | 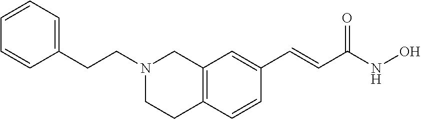 | (E)-N-Hydroxy-3-(2-phenethyl- 1,2,3,4-tetrahydro-isoquinolin-7- yl)-acrylamide | ** | * | 323.1756 |
| 45 | 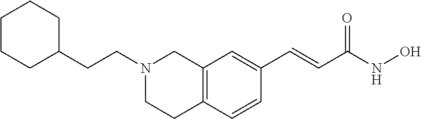 | (E)-3-[2-(2-Cyclohexyl-ethyl)- 1,2,3,4-tetrahydro-isoquinolin-7- yl]-N-hydroxy-acrylamide | ** | * | 329.2229 |
| 46 | 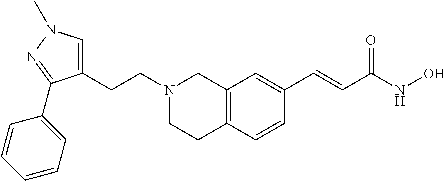 | (E)-N-Hydroxy-3-{2-[2-(1-methyl- 3-phenyl-1H-pyrazol-4-yl)-ethyl]- 1,2,3,4-tetrahydro-isoquinolin-7- yl}-acrylamide | ** | * | 403.2128 |
| 47 | 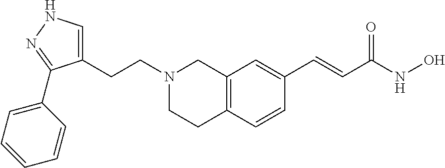 | (E)-N-Hydroxy-3-{2-[2-(3-phenyl- 1H-pyrazol-4-yl)-ethyl]-1,2,3,4- tetrahydro-isoquinolin-7-yl}- acrylamide | ** | * | 389.2 |
| 48 | 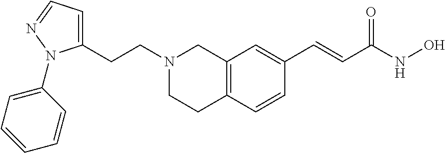 | (E)-N-Hydroxy-3-{2-[2-(2-phenyl- 2H-pyrazol-3-yl)-ethyl]-1,2,3,4- tetrahydro-isoquinolin-7-yl}- acrylamide | ** | * | 389.1972 |
| 49 | 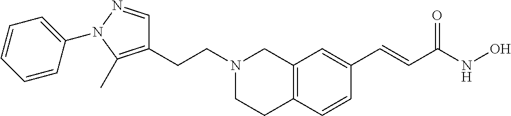 | (E)-N-Hydroxy-3-{2-[2-(5-methyl- 1-phenyl-1H-pyrazol-4-yl)-ethyl]- 1,2,3,4-tetrahydro-isoquinolin-7- yl}-acrylamide | ** | * | 403.1 |
| 50 | 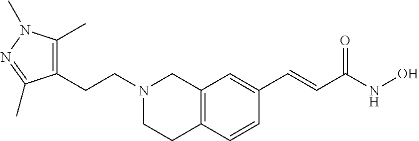 | (E)-N-Hydroxy-3-{2-[2-(1,3,5- trimethyl-1H-pyrazol-4-yl)-ethyl]- 1,2,3,4-tetrahydro-isoquinolin-7- yl}-acrylamide | ** | * | 355.2134 |
| 51 | 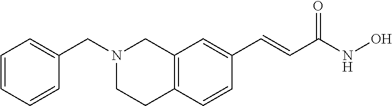 | (E)-3-(2-Benzyl-1,2,3,4-tetrahydro- isoquinolin-7-yl)-N-hydroxy- acrylamide | ** | * | 309.1601 |
| 52 | 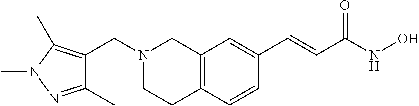 | (E)-N-Hydroxy-3-[2-(1,3,5- trimethyl-1H-pyrazol-4-ylmethyl)- 1,2,3,4-tetrahydro-isoquinolin-7- yl]-acrylamide | ** | * | 341.0 |
| 53 | 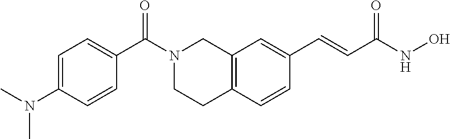 | (E)-3-[2-(4-Dimethylamino- benzoyl)-1,2,3,4-tetrahydro- isoquinolin-7-yl]-N-hydroxy- acrylamide | *** | * | 366.2 |
| 54 | 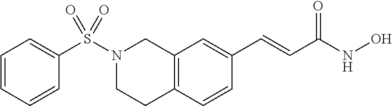 | (E)-3-(2-Benzenesulfonyl-1,2,3,4- tetrahydro-isoquinolin-7-yl)-N- hydroxy-acrylamide | ** | * | 359.0 |
| 55 | 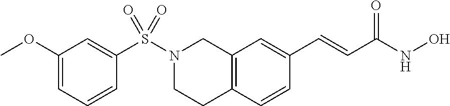 | (E)-N-Hydroxy-3-[2-(3-methoxy- benzenesulfonyl)-1,2,3,4- tetrahydro-isoquinolin-7-yl]- acrylamide | ** | * | 389.1 |
| 56 | 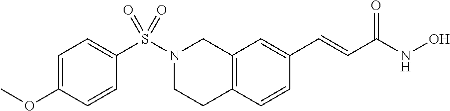 | (E)-N-Hydroxy-3-[2-(4-methoxy- benzenesulfonyl)-1,2,3,4- tetrahydro-isoquinolin-7-yl]- acrylamide | ** | * | 389.0 |
| 57 | 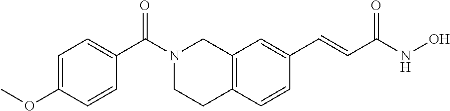 | (E)-N-Hydroxy-3-[2-(4-methoxy- benzoyl)-1,2,3,4-tetrahydro- isoquinolin-7-yl]-acrylamide | *** | * | 353.0 |
| 58 | 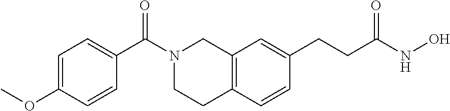 | N-Hydroxy-3-[2-(4-methoxy- benzoyl)-1,2,3,4-tetrahydro- isoquinolin-7-yl]-propionamide | ** | * | 355.1 |
| 59 | 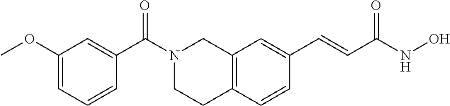 | (E)-N-Hydroxy-3-[2-(3-methoxy- benzoyl)-1,2,3,4-tetrahydro- isoquinolin-7-yl]-acrylamide | ** | * | 353.0 |
| 60 | 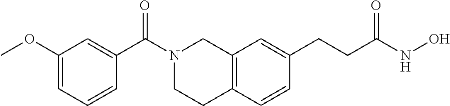 | N-Hydroxy-3-[2-(3-methoxy- benzoyl)-1,2,3,4-tetrahydro- isoquinolin-7-yl]-propionamide | ** | * | 355.1659 |
| 61 | 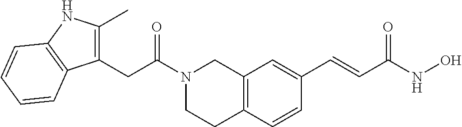 | (E)-N-Hydroxy-3-{2-[2-(2-methyl- 1H-indol-3-yl)-acetyl]-1,2,3,4- tetrahydro-isoquinolin-7-yl}- acrylamide | *** | * | 390.1821 |
| 62 | 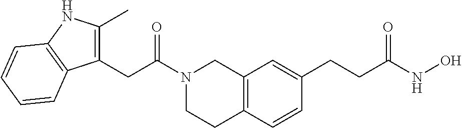 | N-Hydroxy-3-{2-[2-(2-methyl-1H- indol-3-yl)-acetyl]-1,2,3,4- tetrahydro-isoquinolin-7-yl}- propionamide | *** | * | 392.2 |
| 63 | 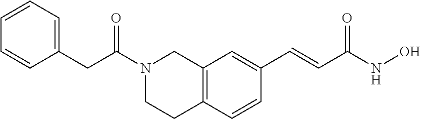 | (E)-N-Hydroxy-3-(2-phenylacetyl- 1,2,3,4-tetrahydro-isoquinolin-7- yl)-acrylamide | *** | * | 337.3 |
| 64 | 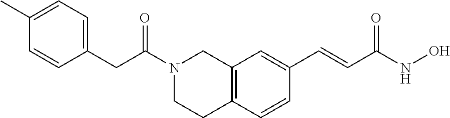 | (E)-N-Hydroxy-3-[2-(2-p-tolyl- acetyl)-1,2,3,4-tetrahydro- isoquinolin-7-yl]-acrylamide | *** | * | 351.1 |
| 65 | 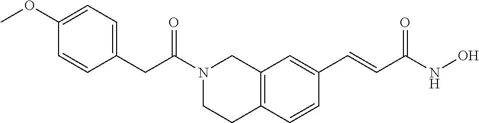 | (E)-N-Hydroxy-3-{2-[2-(4- methoxy-phenyl)-acetyl]-1,2,3,4- tetrahydro-isoquinolin-7-yl}- acrylamide | *** | * | 367.1 |
| 66 | 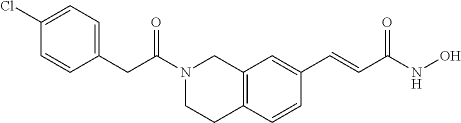 | (E)-3-{2-[2-(4-Chloro-phenyl)- acetyl]-1,2,3,4-tetrahydro- isoquinolin-7-yl}-N-hydroxy- acrylamide | *** | * | 371.1 |
| 67 | 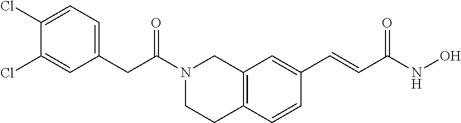 | (E)-3-{2-[2-(3,4-Dichloro-phenyl)- acetyl]-1,2,3,4-tetrahydro- isoquinolin-7-yl}-N-hydroxy- acrylamide | *** | * | 405.1 |
| 68 | 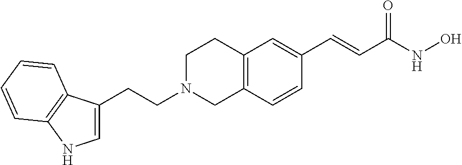 | (E)-N-Hydroxy-3-{2-[2-(1H-indol- 3-yl)-ethyl]-1,2,3,4-tetrahydro- isoquinolin-6-yl}-acrylamide | *** | * | 362.2 |
| 69 | 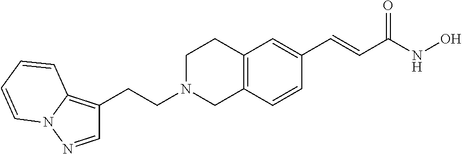 | (E)-N-Hydroxy-3-[2-(2- pyrazolo[1,5-a]pyridin-3-yl-ethyl)- 1,2,3,4-tetrahydro-isoquinolin-6- yl]-acrylamide | *** | * | 363.1 |
| 70 | 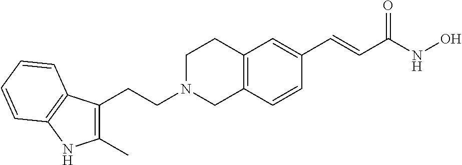 | (E)-N-Hydroxy-3-{2-[2-(2-methyl- 1H-indol-3-yl)-ethyl]-1,2,3,4- tetrahydro-isoquinolin-6-yl}- acrylamide | *** | ** | 376.1999 |
| 71 | 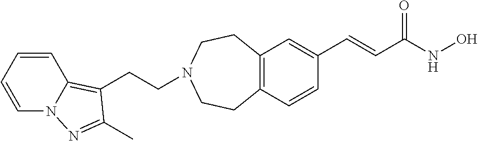 | (E)-N-Hydroxy-3-{3-[2-(2-methyl- pyrazolo[1,5-a]pyridin-3-yl)-ethyl]- 2,3,4,5-tetrahydro-1H- benzo[d]azepin-7-yl}-acrylamide | ** | * | 391.2138 |
| 72 | 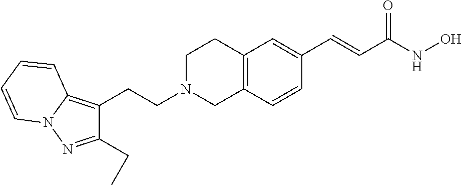 | (E)-3-{2-[2-(2-Ethyl-pyrazolo[1,5- a]pyridin-3-yl)-ethyl]-1,2,3,4- tetrahydro-isoquinolin-6-yl}-N- hydroxy-acrylamide | *** | ** | 391.1 |
| 73 | 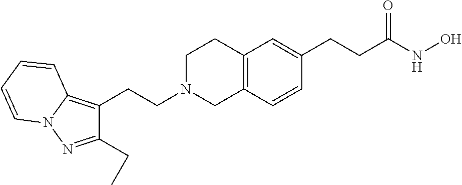 | 3-{2-[2-(2-Ethyl-pyrazolo[1,5- a]pyridin-3-yl)-ethyl]-1,2,3,4- tetrahydro-isoquinolin-6-yl}-N- hydroxy-propionamide | ** | * | 393.2283 |
| 74 | 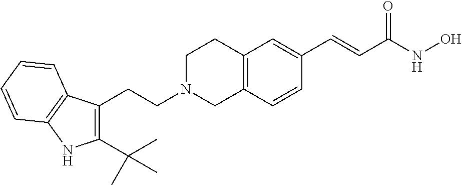 | (E)-3-{2-[2-(2-tert-Butyl-1H-indol- 3-yl)-ethyl]-1,2,3,4-tetrahydro- isoquinolin-6-yl}-N-hydroxy- acrylamide | *** | ** | 418.2493 |
| 75 | 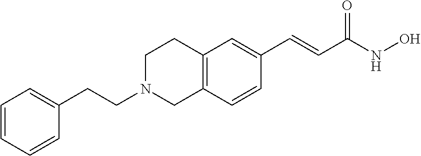 | (E)-N-Hydroxy-3-(2-phenethyl- 1,2,3,4-tetrahydro-isoquinolin-6- yl)-acrylamide | *** | * | 323.1 |
| 76 | 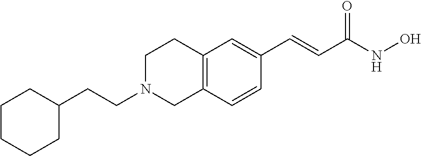 | (E)-3-[2-(2-Cyclohexyl-ethyl)- 1,2,3,4-tetrahydro-isoquinolin-6- yl]-N-hydroxy-acrylamide | *** | * | 329.0 |
| 77 | 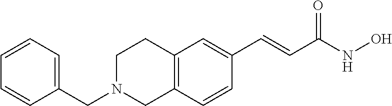 | (E)-3-(2-Benzyl-1,2,3,4-tetrahydro- isoquinolin-6-yl)-N-hydroxy- acrylamide | ** | * | 309.1598 |
| 78 | 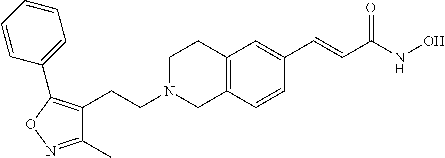 | (E)-N-Hydroxy-3-{2-[2-(3-methyl- 5-phenyl-isoxazol-4-yl)-ethyl]- 1,2,3,4-tetrahydro-isoquinolin-6- yl}-acrylamide | *** | * | 404.1 |
| 79 | 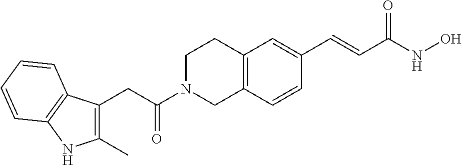 | (E)-N-Hydroxy-3-{2-[2-(2-methyl- 1H-indol-3-yl)-acetyl]-1,2,3,4- tetrahydro-isoquinolin-6-yl}- acrylamide | *** | * | 390.1816 |
| 80 | 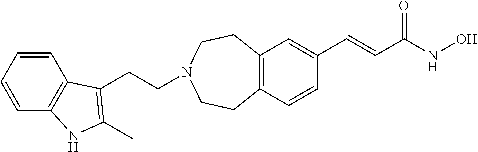 | (E)-N-Hydroxy-3-{3-[2-(2-methyl- 1H-indol-3-yl)-ethyl]-2,3,4,5- tetrahydro-1H-benzo[d]azepin-7- yl}-acrylamide | *** | * | 390.2183 |
| 81 | 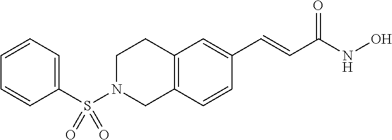 | (E)-3-(2-Benzenesulfonyl-1,2,3,4- tetrahydro-isoquinolin-6-yl)-N- hydroxy-acrylamide | ** | * | 359.0 |
| 82 | 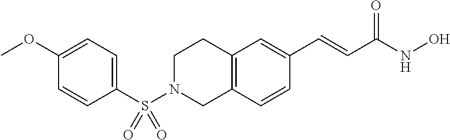 | (E)-N-Hydroxy-3-[2-(4-methoxy- benzenesulfonyl)-1,2,3,4- tetrahydro-isoquinolin-6-yl]- acrylamide | ** | * | 389.0 |
| 83 | 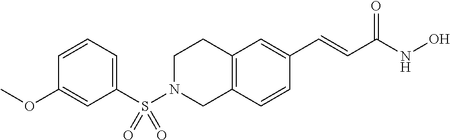 | (E)-N-Hydroxy-3-[2-(3-methoxy- benzenesulfonyl)-1,2,3,4- tetrahydro-isoquinolin-6-yl]- acrylamide | ** | * | 388.9 |
| 84 | 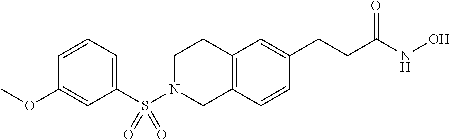 | N-Hydroxy-3-[2-(3-methoxy- benzenesulfonyl)-1,2,3,4- tetrahydro-isoquinolin-6-yl]- propionamide | ** | * | 391.1360 |
| 85 | 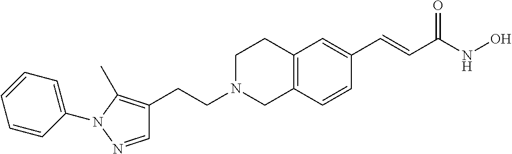 | (E)-N-Hydroxy-3-{2-[2-(5-methyl- 1-phenyl-1H-pyrazol-4-yl)-ethyl]- 1,2,3,4-tetrahydro-isoquinolin-6- yl}-acrylamide | *** | * | 403.0 |
| 86 | 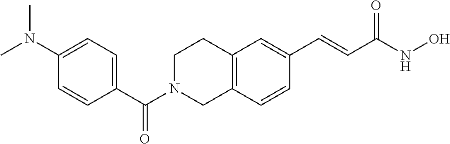 | (E)-3-[2-(4-Dimethylamino- benzoyl)-1,2,3,4-tetrahydro- isoquinolin-6-yl]-N-hydroxy- acrylamide | ** | * | 366.1 |
| 87 | 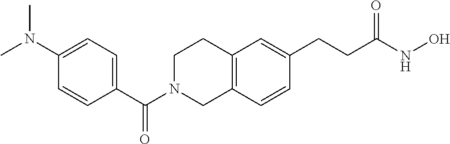 | 3-[2-(4-Dimethylamino-benzoyl)- 1,2,3,4-tetrahydro-isoquinolin-6- yl]-N-hydroxy-propionamide | ** | * | 368.0 |
| 88 | 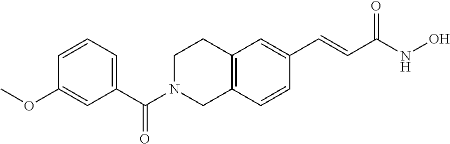 | (E)-N-Hydroxy-3-[2-(3-methoxy- benzoyl)-1,2,3,4-tetrahydro- isoquinolin-6-yl]-acrylamide | ** | * | 353.0 |
| 89 | 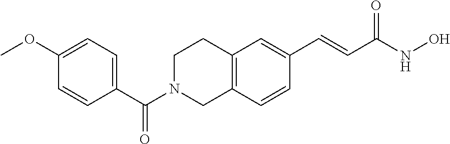 | (E)-N-Hydroxy-3-[2-(4-methoxy- benzoyl)-1,2,3,4-tetrahydro- isoquinolin-6-yl]-acrylamide | ** | * | 353.0 |
| 90 | 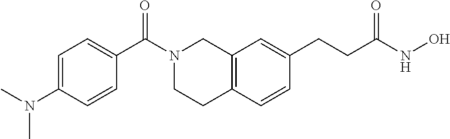 | 3-[2-(4-Dimethylamino-benzoyl)- 1,2,3,4-tetrahydro-isoquinolin-7- yl]-N-hydroxy-propionamide | ** | * | 368.0 |

IC₅₀ KEY
*** IC₅₀ ≦ 100 nM
** IC₅₀ 100 nM to 1 μM
* IC₅₀ ≧ 1 μM

In certain embodiments, a compound of the present invention is further characterized as a modulator of a histone deacetylase (“HDAC”), including a mammalian HDAC, and especially including a human HDAC polypeptide. In a preferred embodiment, the compound of the invention is an HDAC inhibitor.

The terms “HDAC-associated state” or “HDAC-associated disorder” includes disorders and states (e.g., a disease state) that are associated with the activity of HDAC, e.g., deacetylation of histones. HDAC-associated disorders are often associated with abnormal cell growth and abnormal cell proliferation. HDAC-associated states include proliferative diseases, hyperproliferative diseases, diseases of the immune system, diseases of the central nervous system and peripheral nervous system, and diseases associated with misexpression of a gene. An HDAC-associated disorder includes a disease or disorder associated with a mutated HDAC polypeptide, with misregulation of an HDAC polypeptide, or is discovered to respond to inhibition of at least one HDAC polypeptide. The terms “HDAC-associated state” or “HDAC-associated disorder” also include HDAC-dependent diseases.

HDAC-dependent diseases include, e.g., those that depend on activity or misregulation of at least one of HDAC1 (Online Mendelian Inheritance in Man (“OMIM”) accno. 601241), HDAC2, HDAC3 (OMIM accno. 605166), HDAC4 (OMIM accno. 605314), HDAC5 (OMIM accno. 605315), HDAC6, HDAC7, HDAC8 (OMIM accno. 300269), HDAC9 (OMIM accno. 606543), HDAC10 (OMIM accno. 608544), HDAC11 (OMIM accno. 607226), and BRAF35/HDAC complex 80-KD subunit (OMIM accno. 608325), or an HDAC-associated pathway, or a disease dependent on any two or more of the HDACs just mentioned. OMIM is a database of gene-associated diseases maintained by Johns Hopkins University and publicly available through the National Center for Biotechnology Information at the U.S. National Institutes of Health.

The present invention includes treatment of HDAC-associated disorders as described above, but the invention is not intended to be limited to the manner by which the compound performs its intended function of treatment of a disease. The present invention includes treatment of diseases described herein in any manner that allows treatment to occur, e.g., a proliferative disease.

In a related embodiment, the diseases to be treated by the uses and methods of the present invention include diseases and ailments associated with misregulated gene expression. The term “misregulated gene expression” includes altered levels of expression either by increased expression, decreased expression, and includes changes in temporal expression, or a combination thereof, compared to normal.

In embodiments related to these uses and methods, the disease includes a hyperproliferative disease, which includes leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

In certain embodiments, the invention provides a pharmaceutical composition of any of the compounds of the present invention. In a related embodiment, the invention provides a pharmaceutical composition of any of the compounds of the present invention and a pharmaceutically acceptable carrier or excipient of any of these compounds. In certain embodiments, the invention includes the compounds as novel chemical entities.

In one embodiment, the invention includes a packaged HDAC-associated disorder treatment. The packaged treatment includes a compound of the invention packaged with instructions for using an effective amount of the compound of the invention for an intended use.

The compounds of the present invention are suitable as active agents in pharmaceutical compositions that are efficacious particularly for treating deacetylase-associated disorders, including, e.g., cellular proliferative ailments. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially an anti-tumor effect, e.g., inhibition of proliferation of malignant cancer cells, benign tumor cells or other proliferative cells, or of any other deacetylase dependent disease.

In one embodiment, the diseases to be treated by compounds of the invention include, for example, a proliferative disease, preferably a benign or especially malignant tumor, more preferably carcinoma of the brain, kidney, liver, adrenal gland, bladder, breast, stomach (including gastric tumors), esophagus, ovaries, colon, rectum, prostate, pancreas, lung, vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, including those of epithelial character, for example mammary carcinoma, or a leukemia.

In a further embodiment, the disease to be treated is a disease that is triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue.

The compounds of the present invention can also be used for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

In other embodiments, the present invention provides a method for inhibiting a histone deacetylase. The method includes contacting a cell with any of the compounds of the present invention. In a related embodiment, the method further provides that the compound is present in an amount effective to produce a concentration sufficient to selectively inhibit the acetylation of a histone in the cell.

In other embodiments, the present invention provides a use of any of the compounds of the invention for manufacture of a medicament to treat a proliferative or hyperproliferative disease.

In other embodiments, the invention provides a method of manufacture of a medicament, including formulating any of the compounds of the present invention for treatment of a subject.

### DEFINITIONS

The term “treat,” “treated,” “treating” or “treatment” includes the diminishment or alleviation of at least one symptom associated or caused by the state, disorder or disease being treated. In certain embodiments, the treatment comprises the induction of a deacetylase-inhibited state, followed by the activation of the deacetylase-modulating compound, which would in turn diminish or alleviate at least one symptom associated or caused by the deacetylase-associated state, disorder or disease being treated. For example, treatment can be diminishment of one or several symptoms of a disorder or complete eradication of a disorder.

The term “subject” is intended to include organisms, e.g., prokaryotes and eukaryotes, which are capable of suffering from or afflicted with a deacetylase-associated disorder. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a deacetylase-associated disorder, and for diseases or conditions described herein, e.g., proliferative diseases.

The language “deacetylase-modulating compound” refers to compounds that modulate, e.g., inhibit, or otherwise alter, the activity of deacetylase. Examples of deacetylase-modulating compounds include compounds of Formula I and Table A (including pharmaceutically acceptable salts thereof).

Additionally, the method includes administering to a subject an effective amount of a deacetylase modulating compound of the invention, e.g., deacetylase-modulating compounds of Formula I and Table A (including pharmaceutically acceptable salts thereof).

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” also includes alkenyl groups and alkynyl groups. Furthermore, the expression “Cₓ—C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl. Moreover, the term C₃₋₆-cycloalkyl includes, but is not limited to, cyclopropyl, cyclobutyl, cyclopentyl, and cyclohexyl. As discussed below, these alkyl groups, as well as cycloalkyl groups, may be further substituted.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer carbons. Likewise, preferred cycloalkyls have from 4-7 carbon atoms in their ring structure, and more preferably have 5 or 6 carbons in the ring structure.

Moreover, alkyl (e.g., methyl, ethyl, propyl, butyl, pentyl, hexyl, etc.) include both “unsubstituted alkyl” and “substituted alkyl”, the latter of which refers to alkyl moieties having substituents replacing a hydrogen on one or more carbons of the hydrocarbon backbone, which allow the molecule to perform its intended function.

The term “substituted” is intended to describe moieties having substituents replacing a hydrogen on one or more atoms, e.g. C, O or N, of a molecule. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, morpholino, phenol, benzyl, phenyl, piperizine, cyclopentane, cyclohexane, pyridine, 5H-tetrazole, triazole, piperidine, or an aromatic or heteroaromatic moiety.

Further examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. In certain embodiments, a carbonyl moiety (C═O) may be further derivatized with an oxime moiety, e.g., an aldehyde moiety may be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “alkenyl” includes unsaturated aliphatic groups analogous in length and possible substitution to the alkyls described above, but which contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone, such as ketones, aldehydes and imines. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “amine” or “amino” should be understood as being broadly applied to both a molecule, or a moiety or functional group, as generally understood in the art, and may be primary, secondary, or tertiary. The term “amine” or “amino” includes compounds where a nitrogen atom is covalently bonded to at least one carbon, hydrogen or heteroatom. The terms include, for example, but are not limited to, “alkylamino,” “arylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

In a particular embodiment of the invention, the term “amine” or “amino” refers to substituents for the formulas N(R′)R″, CH₂N(R′)R″ and CH(CH₃)N(R′)R″, wherein R′ and R′ are each, independently, selected from the group consisting of —H and —(C₁₋₄alkyl)₀₋₁G, wherein G is selected from the group consisting of —COOH, —H, —PO₃H, —SO₃H, —Br, —Cl, —F, —O—C₁₋₄alkyl, —S—C₁₋₄alkyl, aryl, —C(O)OC₁-C₆-alkyl, —C(O)C₁₋₄alkyl-COOH, —C(O)C₁-C₄-alkyl and —C(O)-aryl.

The term “aryl” includes groups, including 5- and 6-membered single-ring aromatic groups that may include from zero to four heteroatoms, for example, phenyl, pyrrole, furan, thiophene, thiazole, isothiazole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Further examples of alkoxy groups include methanol, ethanol, propanol, iso-propanol and tert-butanol substituents. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties that are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “ester” includes compounds and moieties that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorous.

The term “heterocycle” or “heterocyclic” includes saturated, unsaturated, aromatic (“heteroaryls” or “heteroaromatic”) and polycyclic rings which contain one or more heteroatoms. Examples of heterocycles include, for example, benzodioxazole, benzofuran, benzoimidazole, benzothiazole, benzothiophene, benzoxazole, deazapurine, furan, indole, indolizine, imidazole, isoxazole, isoquinoline, isothiazole, methylenedioxyphenyl, napthridine, oxazole, purine, pyrazine, pyrazole, pyridazine, pyridine, pyrimidine, pyrrole, quinoline, tetrazole, thiazole, thiophene, and triazole. Other heterocycles include morpholino, piprazine, piperidine, thiomorpholino, and thioazolidine. The heterocycles may be substituted or unsubstituted. Examples of substituents include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

It will be noted that the structures of some of the compounds of this invention include asymmetric carbon atoms. It is to be understood accordingly that the isomers arising from such asymmetry (e.g., all enantiomers, stereoisomers, rotamers, tautomers, diastereomers, or racemates) are included within the scope of this invention. Such isomers can be obtained in substantially pure form by classical separation techniques and by stereochemically controlled synthesis. Furthermore, the structures and other compounds and moieties discussed in this application also include all tautomers thereof. Compounds described herein may be obtained through art recognized synthesis strategies.

It will also be noted that the substituents of some of the compounds of this invention include isomeric cyclic structures. It is to be understood accordingly that constitutional isomers of particular substituents are included within the scope of this invention, unless indicated otherwise. For example, the term “tetrazole” includes tetrazole, 2H-tetrazole, 3H-tetrazole, 4H-tetraz+ole and 5H-tetrazole.

In a particular embodiment of the invention, the deacetylase modulating compound of formula (I) is any one of the compounds of Table A.

In yet another embodiment, the invention pertains to pharmaceutical compositions comprising the deacetylase-modulating compounds described herein and a pharmaceutical acceptable carrier.

### Use in Deacetylase-Dependent Diseases

The compounds of the present invention have valuable pharmacological properties and are useful in the treatment of diseases. In certain embodiments, compounds of the invention are useful in the treatment of deacetylase-associated disorders, e.g., as drugs to treat proliferative diseases.

The term “use” includes any one or more of the following embodiments of the invention, respectively: the use in the treatment of deacetylase dependent diseases; the use for the manufacture of pharmaceutical compositions for use in the treatment of these diseases, e.g., in the manufacture of a medicament; methods of use of aminoalkyl derivatives in the treatment of these diseases; pharmaceutical preparations having aminoalkyl derivatives for the treatment of these diseases; and aminoalkyl derivatives for use in the treatment of these diseases; as appropriate and expedient, if not stated otherwise. In particular, diseases to be treated and are thus preferred for use of a compound of the present invention are selected from deacetylase-associated disorders, including those corresponding proliferative diseases, and those diseases that depend on HDAC1, HDAC2, HDAC3, HDAC4, HDAC5, HDAC6, HDAC7, HDAC8, HDAC9, HDAC10, HDAC11, or any combinations thereof, or an HDAC complex (hereinafter “HDACs”) can therefore be used in the treatment of deacetylase dependent diseases. The term “use” further includes embodiments of compositions herein which bind to a deacetylase protein sufficiently to serve as tracers or labels, so that when coupled to a fluorous tag, fluorine tag, or made radioactive, can be used as a research reagent or as a diagnostic or an imaging agent.

In certain embodiments, a compound of the present invention is used for treating deacetylase-associated diseases, i.e., a disease associated with the activity of at least one of the deacetylases as described herein, and use of the compound of the present invention as an inhibitor of any one or more deacetylases. It is envisioned that a use can be a treatment of inhibiting one or a subset of deacetylases, including one or a subset of deacetylases in the group HDAC1, HDAC2, HDAC3, HDAC4, HDAC5, HDAC6, HDAC7, HDAC8, HDAC9, HDAC10, and HDAC11, and does not imply that all of these enzymes are inhibited to an equal extent by any of the compounds herein.

Various embodiments of the compounds of the present invention have valuable pharmacological properties and are useful in the treatment of protein deacetylase-associated diseases, e.g., as drugs to treat proliferative and hyperproliferative diseases, and other deacetylase-associated diseases as listed throughout this disclosure.

### Assays

The inhibition of deacetylase may be measured using the assay described below in the “Biological Activity” section. The inhibition of deacetylase activity may also be measured as follows: The baculovirus donor vector pFB-GSTX3 is used to generate a recombinant baculovirus that expresses the deacetylase polypeptide. Transfer vectors containing the deacetylase coding region are transfected into the DH10Bac cell line (GIBCO) and plated on selective agar plates. Colonies without insertion of the fusion sequence into the viral genome (carried by the bacteria) are blue. Single, white colonies are picked and viral DNA (bacmid) are isolated from the bacteria by standard plasmid purification procedures. Sf9 cells or Sf21 (American Type Culture Collection) cells are then transfected in 25 cm² flasks with the viral DNA using Cellfectin reagent.

Determination of small scale protein expression in Sf9 cells: Virus-containing media is collected from the transfected cell culture and used for infection to increase its titer. Virus-containing media obtained after two rounds of infection is used for large-scale protein expression. For large-scale protein expression 100 cm² round tissue culture plates are seeded with 5×10⁷ cells/plate and infected with 1 mL of virus-containing media (at an approximately MOI of 5). After 3 days, the cells are scraped off the plate and centrifuged at 500 rpm for 5 minutes. Cell pellets from 10-20, 100 cm² plates, are re-suspended in 50 mL of ice-cold lysis buffer (25 mM tris-HCl, pH 7.5, 2 mM EDTA, 1% NP-40, 1 mM DTT, 1 mM P MSF). The cells are stirred on ice for 15 minutes and then centrifuged at 5,000 rpms for 20 minutes.

Purification of GST-tagged proteins: The centrifuged cell lysate is loaded onto a 2 mL glutathione-sepharose column (Pharmacia) and is washed 3× with 10 mL of 25 mM tris-HCl, pH 7.5, 2 mM EDTA, 1 mM DTT, 200 mM NaCl. The GST-tagged proteins are then eluted by 10 applications (1 mL each) of 25 mM tris-HCl, pH 7.5, 10 mM reduced-glutathione, 100 mM NaCl, 1 mM DTT, 10% glycerol and stored at −70° C.

Measure of enzyme activity: Deacetylase assays with purified GST-deacetylase protein, such as a GST-HDAC protein, are carried out in a final volume of 30 μL containing 15 ng of GST-deacetylase protein, 20 mM tris-HCl, pH 7.5, 1 mM MnCl2, 10 mM MgCl2, 1 mM DTT, 3 μg/mL poly(Glu, Tyr) 4:1, 1% DMSO, 2.0 μM ATP(γ-[³³P]-ATP 0.1 μCi). The activity is assayed in the presence or absence of inhibitors. The assay is carried out in 96-well plates at ambient temperature for 15 minutes under conditions described below and terminated by the addition of 20 μL of 125 mM EDTA. Subsequently, 40 μL of the reaction mixture are transferred onto IMMOBILON-PVDF membrane (Millipore) previously soaked for 5 minutes with methanol, rinsed with water, then soaked for 5 minutes with 0.5% H₃PO₄ and mounted on vacuum manifold with disconnected vacuum source. After spotting all samples, a vacuum is connected and each well-rinsed with 200 μL 0.5% H₃PO₄. Membranes are removed and washed 4× on a shaker with 1.0% H₃PO₄, once with ethanol. Membranes are counted after drying at ambient temperature, mounting in Packard TopCount 96-well frame, and addition of 10 μL/well of MICROSCINT TM (Packard). IC₅₀ values are calculated by linear regression analysis of the percentage inhibition of each compound in duplicate, at 4 concentrations (usually 0.01, 0.1, 1 and 10 μM).

IC₅₀ Calculations:

Input: 3×4 μL stopped assay on IMMOBILON membrane, not washed

background (3 wells): assay with H₂O instead of enzyme

positive control (4 wells): 3% DMSO instead of compound

bath control (1 well): no reaction mix

IC₅₀ values are calculated by logarithmic regression analysis of the percentage inhibition of each compound at 4 concentrations (usually 3- or 10-fold dilution series starting at 10 μM). In each experiment, the actual inhibition by reference compound is used for normalization of IC₅₀ values to the basis of an average value of the reference inhibitor:

Normalized IC₅₀=measured IC₅₀ average ref. IC₅₀/measured ref. IC₅₀

Example: Reference inhibitor in experiment 0.4 μM, average 0.3 μM

Test compound in experiment 1.0 μM, normalization: 0.3/0.4=0.75 μM

For example, known deacetylase inhibitors or a synthetic derivative thereof may be used as reference compounds.

### Proliferative Diseases

As discussed above, the compounds of the present invention are useful for treating proliferative diseases. A proliferative disease includes, for example, a tumor disease (or cancer) and/or any metastases). The inventive compounds are useful for treating a tumor which is, for example, a breast cancer, genitourinary cancer, lung cancer, gastrointestinal cancer, esophageal cancer, epidermoid cancer, melanoma, ovarian cancer, pancreas cancer, neuroblastoma, head and/or neck cancer or bladder cancer, or in a broader sense renal, brain or gastric cancer; including (i) a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor (including a hormone-refractory prostate tumor); or (ii) a proliferative disease that is refractory to the treatment with other chemotherapeutics; or (iii) a tumor that is refractory to treatment with other chemotherapeutics due to multidrug resistance.

TABLE 1
HDAC 1-11 genes with O.M.I.M accession
number and chromosomal locus

| Histone Deacetylase | OMIM accession number | Chromosomal locus |
|---|---|---|
| HDAC1 | *601241 | 1p34.1 |
| HDAC2 | *605164 | 6q21 |
| HDAC3 | *605166 | 5q31 |
| HDAC4 | *605314 | 2q37.2 |
| HDAC5 | *605315 | Chr.17 |
| HDAC6 | *300272 | Xp11.23 |
| HDAC7A | *606542 | Chr.12 |
| HDAC8 | *300629 | Xq13 |
| HDAC9 | *606543 | 7p21-p15 |
| HDAC10 | *608544 | 22q13.31-q13.33 |
| HDAC11 | *607226 | 3p25.2 |

A deacetylase-associated disorder includes any pathology related to expression of one or more of the genes encoding one of the deacetylase proteins or deacetylase-associated proteins, or an activity of such protein, in that inhibition of the protein results in remediation of the pathology. The deacetylase genes and proteins are as described in the Online Mendelian Inheritance in Man (O.M.I.M). Inhibition of an HDAC protein provides remediation of an HDAC dependent disease. Table 1 lists the HDAC proteins and the locus of each on the human genome. Table 2 shows HDAC 1-11 GenBank accession numbers for representative amino acid sequences in at least three organismal species when available.

TABLE 2
GenBank accession numbers for exemplary
amino acid sequences of HDAC1-11 proteins

| | GenBank amino acid | |
|---|---|---|
| Histone Deacetylase | sequence accession | |
| protein | number | Source |
| HDAC1 | O60341 | Human |
| | NP_033214 | Mouse |
| | NP_571138 | Zebra fish |
| HDAC2 | NP_032255 | Human |
| | P70288 | Mouse |
| HDAC3 | NP_006302 | Human |
| | NP_034541 | Mouse |
| | NP_957284 | Zebra fish |
| HDAC4 | NP_005648 | Human |
| | NP_989644 | Chicken |
| | AAX52490 | Fruit fly |
| HDAC5 | NP_001015033 | Human |
| | AAS77826 | Porcine |
| | NP_034542 | Mouse |
| HDAC6 | Q9C2B2 | Human |
| | NP_034543 | Mouse |
| | AAH43813 | African clawed frog |
| HDAC7 | NP_057680 | Human |
| | AAK11188 | Norway rat |
| | Q8C2B3 | Mouse |
| HDAC8 | Q9BY41 | Human |
| | Q8VH37 | Mouse |
| | AAH55541 | Zebra fish |
| HDAC9 | Q9UKV0 | Human |
| | NP_07738 | Mouse |
| | NP_957110 | Zebra fish |
| HDAC10 | Q969S8 | Human |
| | Q569C4 | Norway rat |
| | NP_954668 | Mouse |
| HDAC11 | Q96DB2 | Human |
| | Q91WA3 | Mouse |

In certain embodiments, the proliferative disease may furthermore be a hyperproliferative condition such as leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

Where a tumor, a tumor disease, a carcinoma or a cancer are mentioned, also metastasis in the original organ or tissue and/or in any other location are implied alternatively or in addition, whatever the location of the tumor and/or metastasis.

In one embodiment, the compounds described herein are selectively toxic or more toxic to rapidly proliferating cells than to normal cells, including, for example, human cancer cells, e.g., cancerous tumors, the compounds have significant antiproliferative effects and promotes differentiation, e.g., cell cycle arrest and apoptosis. In addition, the compounds induce p21, cyclin-CDK interacting protein, which induces either apoptosis or G1 arrest in a variety of cell lines.

In embodiments described herein, general expression can be replaced by the corresponding more specific definitions provided above and below.

In certain embodiments, the use of compounds of the present invention, tautomers thereof or pharmaceutically acceptable salts thereof, where the deacetylase-associated disorder to be treated is a proliferative disease depending on any one or more of the following deacetylases, including, for example, HDAC1, HDAC2, HDAC6 and HDAC8.

In other embodiments, the deacetylase dependant disease may be a proliferative disease including a hyperproliferative condition, such as leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

In other embodiments, the invention provides a method of treating a deacetylase-associated disorder comprising administering a compound of the present invention, where the disease to be treated is a proliferative disease, including, for example, a benign or malignant tumor, a carcinoma of the brain, kidney, liver, adrenal gland, bladder, breast, stomach (including gastric tumors), esophagus, ovaries, colon, rectum, prostate, pancreas, lung (including SCLC), vagina, thyroid, sarcoma, glioblastomas, multiple myeloma or gastrointestinal cancer, especially colon carcinoma or colorectal adenoma, or a tumor of the neck and head, an epidermal hyperproliferation, including psoriasis, prostate hyperplasia, a neoplasia, including those of epithelial character, including mammary carcinoma, or a leukemia. Also included is a method for the treatment of atherosclerosis, thrombosis, psoriasis, scleroderma and fibrosis.

Compounds of the present invention are able to slow tumor growth, stop tumor growth, or bring about the regression of tumors and to prevent the formation of tumor metastases (including micrometastases) and the growth of metastases (including micrometastases). In addition they can be used in epidermal hyperproliferation (e.g., psoriasis), in prostate hyperplasia, and in the treatment of neoplasias, including that of epithelial character, for example mammary carcinoma. It is also possible to use the compounds of the present invention in the treatment of diseases of the immune system insofar as one or more individual deacetylase protein species or associated proteins are involved. Furthermore, the compounds of the present invention can be used also in the treatment of diseases of the central or peripheral nervous system where signal transmission by at least one deacetylase protein is involved.

Deacetylase inhibitors are also appropriate for the therapy of diseases related to transcriptional regulation of proteins involved in signal transduction, such as VEGF receptor tyrosine kinase overexpression. Among these diseases are retinopathies, age-related macula degeneration, psoriasis, haemangioblastoma, haemangioma, arteriosclerosis, muscle wasting conditions such as muscular dystrophies, cachexia, Huntington's syndrome, inflammatory diseases such as rheumatoid or rheumatic inflammatory diseases, including arthritis and arthritic conditions, such as osteoarthritis and rheumatoid arthritis, or other chronic inflammatory disorders such as chronic asthma, arterial or post-transplantational atherosclerosis, endometriosis, and especially neoplastic diseases, for example so-called solid tumors (including cancers of the gastrointestinal tract, the pancreas, breast, stomach, cervix, bladder, kidney, prostate, esophagus, ovaries, endometrium, lung, brain, melanoma, Kaposi's sarcoma, squamous cell carcinoma of head and neck, malignant pleural mesotherioma, lymphoma or multiple myeloma) and liquid tumors (e.g., leukemias).

HDAC proteins share a set of nine consensus sequences. HDAC proteins are classified into two classes based on amino acid sequence: class I proteins such as HDAC1, HDAC2 and HDAC3 have substantial homology to yeast Rpd3; class II such as HDAC4 and HDAC6 show homology to yeast Hda1. Various facts indicate an association of these proteins with the HDAC dependent diseases.

HDAC1 is a protein having 482 amino acids, and is highly conserved in nature, having 60% identity to a yeast transcription factor. It is found at various levels in all tissues, and is involved in transcriptional regulation and cell cycle progression, particularly G1 checkpoint control. HDAC1 interacts physically with and cooperates with RB1, the retinoblastoma tumor suppressor protein that inhibits cell proliferation, and with nuclear transcription factor NFκB.

HDAC2 is also known as YY1-associated factor (YAF1), as it associates with mammalian zinc finger transcription factor YY1. The locus that encodes this protein on the human genome is 6q21, a region of the genome implicated in childhood acute lymphocytic leukemia (ALL) and ulnar ray limb defect. Further, HDAC2 interacts with and is physically associated with BRCA1 in a complex that includes also HDAC1. The common core of this complex functions to repress genes to a silent condition. A different complex is formed during S phase, and histone is deacetylated into heterochromatin following replication.

HDAC3 is known to be expressed in all human tissues and tumor cell lines. Transfection of a human myeloid leukemia line resulted in accumulation of cells at the G2/M boundary phase with aberrant nuclear morphology and increased cell size. The catalytic domain of HDAC4 interacts with HDAC3.

HDAC4 deacetylase activity acts on all four core histone proteins, and is expressed in prehypertrophic chondrocytes and regulates chondrocyte hypertrophy, endochondral bone formation and skeletogenesis. HDAC4-null mice display premature ossification. With MIR and CABIN1, HDAC4 constitutes a family of calcium-sensitive transcriptions repressors of MEF-2 (myocyte enhancer factor-2).

HDAC5 is expressed in all tissues tested, with lower expression in spleen and pancreas. The 1,123 amino acid sequence of HDAC5 is 51% identical to HDAC4. Five of 29 colon cancer patients tested serologically positive for antibody to HDAC5. MEF-2 protein interacts with HDAC4 and HDAC5.

HDAC6 is a tubulin deacetylase and is localized exclusively in cytoplasm. This enzyme has potent deacetylase activity for assembled microtubules and therapeutic intervention into its expression or activity can be associated with a variety of conditions affecting muscle integrity and muscle wasting, such as Huntington's disease and cachexia. HDAC is also a tubulin and heat shock protein (Hsp90) inhibitor.

HDAC7A transcript is found predominantly in heart and lung tissues, and to a lesser extent in skeleton muscle. The protein co-localizes with HDAC5 in subnuclear regions.

HDAC8 is a 377 amino acid protein which while possessing the typical nine conserved HDAC blocks of consensus sequence, has sequences at each of the amino and carboxy termini that are distinct from those of other HDAC proteins. It is expressed most strongly in brain. Knockdown of expression by RNAi inhibits growth of human lung, colon, and cervical cancer cell lines. The map position of the encoding gene at Xq 13 is located near XIST which is involved in initiation of X chromosome inactivation, and near breakpoints associated with preleukemia conditions. Further, therapeutic intervention into its expression or activity can be associated with a variety of conditions affecting inflammatory diseases such as various arthritic conditions, e.g., rheumatoid arthritis.

HDAC9 is known also as 7B, MITR, and KIAA0744. It is expressed most actively in brain, and to a lesser extent in heart and smooth muscle, and very little in other tissues. This protein interacts with HDAC1 and is a repressor of transcription. A longer isoform contains 1,011 amino acids and a shorter form, known as 9a, contains 879 amino acids, lacking 132 residues at the C-terminus, predominates in lung, liver and skeletal muscle.

HDAC10 is found in two splice variants of 669 and 649 amino acids. The protein represses transcription from a thymidine kinase promoter and interacts with HDAC3.

HDAC11 is a 347 amino acid protein that is expressed most highly in brain, heart, skeletal muscle, kidney and testis. It partitions with nuclear extracts.

Angiogenesis is believed to involve tumor growth beyond a maximum diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size.

Three principal mechanisms play an important part in the activity of angiogenesis inhibitors against tumors: 1) Inhibition of the growth of vessels, especially capillaries, into avascular resting tumors, with the result that there is no net tumor growth owing to the balance that is achieved between apoptosis and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells which normally line the vessels.

The present invention can also be used to prevent or treat diseases that are triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

### Pharmaceutical Compositions

The language “effective amount” of the compound is that amount necessary or sufficient to treat or prevent a deacetylase-associated state, e.g. prevent the various morphological and somatic symptoms of a deacetylase-associated disorder, and/or a disease or condition described herein. In an example, an effective amount of the deacetylase-modulating compound is the amount sufficient to inhibit undesirable cell growth in a subject. In another example, an effective amount of the deacetylase-modulating compound is the amount sufficient to reduce the size of a pre-existing benign cell mass or malignant tumor in a subject. The effective amount can vary depending on such factors as the size and weight of the subject, the type of illness, or the particular compound of the invention. For example, the choice of the compound of the invention can affect what constitutes an “effective amount”. One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the onset of a deacetylase-associated state. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation.

Compounds of the invention may be used in the treatment of states, disorders or diseases as described herein, or for the manufacture of pharmaceutical compositions for use in the treatment of these diseases. Methods of use of compounds of the present invention in the treatment of these diseases, or pharmaceutical preparations having compounds of the present invention for the treatment of these diseases.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats a deacetylase-associated disorder.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### Synthetic Procedure

Compounds of the present invention are prepared from commonly available compounds using procedures known to those skilled in the art, including any one or more of the following conditions without limitation:

Within the scope of this text, a readily removable group that is not a constituent of the particular desired end product of the compounds of the present invention is designated a “protecting group.” The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Salts of compounds of the present invention having at least one salt-forming group may be prepared in a manner known per se. For example, salts of compounds of the present invention having acid groups may be formed, for example, by treating the compounds with metal compounds, such as alkali metal salts of suitable organic carboxylic acids, e.g., the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g., by treating the compounds with an acid or a suitable anion exchange reagent. Internal salts of compounds of the present invention containing acid and basic salt-forming groups, e.g., a free carboxy group and a free amino group, may be formed, e.g., by the neutralisation of salts, such as acid addition salts, to the isoelectric point, e.g., with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

### General Process Conditions

The following applies in general to all processes mentioned throughout this disclosure.

The process steps to synthesize the compounds of the invention can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g., in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany. 2005.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane, heptane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

### Prodrugs

This invention also encompasses pharmaceutical compositions containing, and methods of treating deacetylase-associated states, through administering pharmaceutically acceptable prodrugs of compounds of the compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

Any reference to a compound of the present invention is therefore to be understood as referring also to the corresponding pro-drugs of the compound of the present invention, as appropriate and expedient.

### Combinations

A compound of the present invention may also be used to advantage in combination with other antiproliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors (e.g., velcade or gemcitabine); agents used in the treatment of hematologic malignancies; compounds which target, decrease or inhibit the activity of Flt-3; Hsp90 inhibitors; temozolomide (TEMODAL®); and leucovorin.

The phrase, “aromatase inhibitor” as used herein relates to a compound which inhibits the estrogen production, i.e., the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g., under the trademark AROMASIN. Formestane can be administered, e.g., in the form as it is marketed, e.g., under the trademark LENTARON. Fadrozole can be administered, e.g., in the form as it is marketed, e.g., under the trademark AFEMA. Anastrozole can be administered, e.g., in the form as it is marketed, e.g., under the trademark ARIMIDEX. Letrozole can be administered, e.g., in the form as it is marketed, e.g., under the trademark FEMARA or FEMAR. Aminoglutethimide can be administered, e.g., in the form as it is marketed, e.g., under the trademark ORIMETEN. A combination of the invention comprising a chemotherapeutic agent which is an aromatase inhibitor is particularly useful for the treatment of hormone receptor positive tumors, e.g., breast tumors.

The term “antiestrogen” as used herein relates to a compound that antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g., under the trademark NOLVADEX. Raloxifene hydrochloride can be administered, e.g., in the form as it is marketed, e.g., under the trademark EVISTA. Fulvestrant can be formulated as disclosed in U.S. Pat. No. 4,659,516 or it can be administered, e.g., in the form as it is marketed, e.g., under the trademark FASLODEX. A combination of the invention comprising a chemotherapeutic agent which is an antiestrogen is particularly useful for the treatment of estrogen receptor positive tumors, e.g., breast tumors.

The term “anti-androgen” as used herein relates to any substance which is capable of inhibiting the biological effects of androgenic hormones and includes, but is not limited to, bicalutamide (CASODEX), which can be formulated, e.g., as disclosed in U.S. Pat. No. 4,636,505.

The phrase, “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g., under the trademark ZOLADEX. Abarelix can be formulated, e.g., as disclosed in U.S. Pat. No. 5,843,901.

The phrase, “topoisomerase I inhibitor” as used herein includes, but is not limited to topotecan, gimatecan, irinotecan, camptothecan and its analogues, 9-nitrocamptothecin and the macromolecular camptothecin conjugate PNU-166148 (compound A1 in WO99/17804). Irinotecan can be administered, e.g., in the form as it is marketed, e.g., under the trademark CAMPTOSAR. Topotecan can be administered, e.g., in the form as it is marketed, e.g., under the trademark HYCAMTIN.

The phrase, “topoisomerase II inhibitor” as used herein includes, but is not limited to the anthracyclines such as doxorubicin (including liposomal formulation, e.g., CAELYX), daunorubicin, epirubicin, idarubicin and nemorubicin, the anthraquinones mitoxantrone and losoxantrone, and the podophyllotoxins etoposide and teniposide. Etoposide can be administered, e.g., in the form as it is marketed, e.g., under the trademark ETOPOPHOS. Teniposide can be administered, e.g., in the form as it is marketed, e.g., under the trademark VM 26-BRISTOL. Doxorubicin can be administered, e.g., in the form as it is marketed, e.g., under the trademark ADRIBLASTIN or ADRIAMYCIN. Epirubicin can be administered, e.g., in the form as it is marketed, e.g., under the trademark FARMORUBICIN. Idarubicin can be administered, e.g., in the form as it is marketed, e.g., under the trademark ZAVEDOS. Mitoxantrone can be administered, e.g., in the form as it is marketed, e.g., under the trademark NOVANTRON.

The phrase, “microtubule active agent” relates to microtubule stabilizing, microtubule destabilizing agents and microtublin polymerization inhibitors including, but not limited to taxanes, e.g., paclitaxel and docetaxel, vinca alkaloids, e.g., vinblastine, including vinblastine sulfate, vincristine including vincristine sulfate, and vinorelbine, discodermolides, cochicine and epothilones and derivatives thereof, e.g., epothilone B or D or derivatives thereof. Paclitaxel may be administered e.g., in the form as it is marketed, e.g., TAXOL. Docetaxel can be administered, e.g., in the form as it is marketed, e.g., under the trademark TAXOTERE. Vinblastine sulfate can be administered, e.g., in the form as it is marketed, e.g., under the trademark VINBLASTIN R.P. Vincristine sulfate can be administered, e.g., in the form as it is marketed, e.g., under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Included are Epothilone A and/or B.

The phrase, “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g., under the trademark CYCLOSTIN. Ifosfamide can be administered, e.g., in the form as it is marketed, e.g., under the trademark HOLOXAN.

The phrase, “histone deacetylase inhibitors” or “HDAC inhibitors” relates to compounds which inhibit at least one example of the class of enzymes known as a histone deacetylase, as described herein, and which compounds generally possess antiproliferative activity. Previously disclosed HDAC inhibitors include compounds disclosed in, e.g., WO 02/22577, including N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further includes Suberoylanilide hydroxamic acid (SAHA). Other publicly disclosed HDAC inhibitors include butyric acid and its derivatives, including sodium phenylbutyrate, thalidomide, trichostatin A and trapoxin.

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate, and folic acid antagonists such as pemetrexed. Capecitabine can be administered, e.g., in the form as it is marketed, e.g., under the trademark XELODA. Gemcitabine can be administered, e.g., in the form as it is marketed, e.g., under the trademark GEMZAR. Also included is the monoclonal antibody trastuzumab which can be administered, e.g., in the form as it is marketed, e.g., under the trademark HERCEPTIN.

The phrase, “platin compound” as used herein includes, but is not limited to, carboplatin, cis-platin, cisplatinum and oxaliplatin. Carboplatin can be administered, e.g., in the form as it is marketed, e.g., under the trademark CARBOPLAT. Oxaliplatin can be administered, e.g., in the form as it is marketed, e.g., under the trademark ELOXATIN.

The phrase, “compounds targeting/decreasing a HDAC activity; or a histone deacetylase activity; or further anti-angiogenic compounds” as used herein includes, but is not limited to: HDAC1-11 inhibitors, e.g.: HDAC2, HDAC3 AND HDAC8 inhibitors.

The following list of proteins involved in signal transduction illustrates far reaching effects of modulating transcription by inhibiting HDAC activity:

i) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g., a N-phenyl-2-pyrimidine-amine derivative, e.g., imatinib, SU101, SU6668, and GFB-111;

ii) compounds targeting, decreasing or inhibiting the activity of the fibroblast growth factor-receptors (FGFR);

iii) compounds targeting, decreasing or inhibiting the activity of the insulin-like growth factor receptor I(IGF-IR), such as compounds which target, decrease or inhibit the activity of IGF-IR, especially compounds which inhibit the IGF-IR receptor, such as those compounds disclosed in WO 02/092599; and/or

iv) compounds targeting, decreasing or inhibiting the activity of the c-Met receptor.

Tumor cell damaging approaches refer to approaches such as ionizing radiation. The phrase, “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See, e.g., Hellman, Principles of Radiation Therapy, Cancer, in Principles and Practice of Oncology, Devita et al., Eds., 4th Edition, Vol. 1, pp. 248-275 (1993).

The phrase, “EDG binders” as used herein refers a class of immunosuppressants that modulates lymphocyte recirculation, such as FTY720.

CERTICAN (everolimus, RAD) an investigational novel proliferation signal inhibitor that prevents proliferation of T-cells and vascular smooth muscle cells.

The phrase, “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindoline-1,3-dione derivatives, such as PL-1, PL-2, PL-3, PL-4, PL-5, PL-6, PL-7 or PL-8 mentioned in Nandy et al, Acta Oncologica, Vol. 33, No. 8, pp. 953-961 (1994).

The phrase, “S-adenosylmethionine decarboxylase inhibitors” as used herein includes, but is not limited to the compounds disclosed in U.S. Pat. No. 5,461,076.

Also included are in particular those compounds, proteins or monoclonal antibodies of VEGF disclosed in WO 98/35958, e.g., 1-(4-chloroanilino)-4-(4-pyridylmethyl)phthalazine or a pharmaceutically acceptable salt thereof, e.g., the succinate, or in WO 00/09495, WO 00/27820, WO 00/59509, WO 98/11223, WO 00/27819 and EP 0 769 947; those as described by Prewett et al, Cancer Res, Vol. 59, pp. 5209-5218 (1999); Yuan et al, Proc Natl Acad Sci USA, Vol. 93, pp. 14765-14770 (1996); Zhu et al., Cancer Res, Vol. 58, pp. 3209-3214 (1998); and Mordenti et al., Toxicol Pathol, Vol. 27, No. 1, pp. 14-21 (1999); in WO 00/37502 and WO 94/10202; ANGIOSTATIN, described by O'Reilly et al., Cell, Vol. 79, pp. 315-328 (1994); ENDOSTATIN, described by O'Reilly et al., Cell, Vol. 88, pp. 277-285 (1997); anthranilic acid amides; ZD4190; ZD6474; SU5416; SU6668; bevacizumab; or anti-VEGF antibodies or anti-VEGF receptor antibodies, e.g., rhuMAb and RHUFab, VEGF aptamer e.g., Macugon; FLT-4 inhibitors, FLT-3 inhibitors, VEGFR-2 IgG1 antibody, Angiozyme (RPI 4610) and Avastan.

Photodynamic therapy as used herein refers to therapy that uses certain chemicals known as photosensitizing agents to treat or prevent cancers. Examples of photodynamic therapy include treatment with agents, such as e.g., VISUDYNE and porfimer sodium.

The phrase, “angiostatic steroids” as used herein refers to agents which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to agents, such as e.g., fluocinolone, dexamethasone.

Other chemotherapeutic agents include, but are not limited to, plant alkaloids, hormonal agents and antagonists; biological response modifiers, preferably lymphokines or interferons; antisense oligonucleotides or oligonucleotide derivatives; or miscellaneous agents or agents with other or unknown mechanism of action.

The structure of the active agents identified by code numbers, generic or trade names may be taken from the actual edition of the standard compendium “The Merck Index” or from databases, e.g., Patents International (e.g., IMS World Publications).

The above-mentioned compounds, which can be used in combination with a compound of the present invention, can be prepared and administered as described in the art such as in the documents cited above.

A compound of the present invention may also be used to advantage in combination with known therapeutic processes, e.g., the administration of hormones or especially radiation.

A compound of the present invention may in also be used as a radiosensitizer, including, for example, the treatment of tumors which exhibit poor sensitivity to radiotherapy.

By the term “combination”, is meant either a fixed combination in one dosage unit form, or a kit of parts for the combined administration where a compound of the present invention and a combination partner may be administered independently at the same time or separately within time intervals that especially allow that the combination partners show a cooperative, e.g., synergistic, effect, or any combination thereof.

### Exemplification of the Invention

The invention is further illustrated by the following examples, which should not be construed as further limiting. The assays used throughout the Examples are accepted. Demonstration of efficacy in these assays is predictive of efficacy in subjects.

### General Synthesis Methods

All starting materials, building blocks, reagents, acids, bases, dehydrating agents, solvents, and catalysts utilized to synthesize the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

### Preparation of Aryl Bromide Heterocyclic Building Blocks

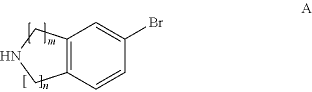

### 5-Bromo-2,3-dihydro-1H-isoindoline

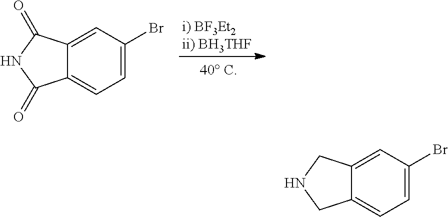

To a solution of 5-bromophthalimide (44.2 mmol; 1.0 equiv) in THF (221 mL) under N₂ atmosphere is added BF₃—OEt₂ (265.5 mmol; 6.0 equiv) and the reaction is stirred for 30 minutes at 25 C. BH₃-THF (353.6 mmol; 8.0 equiv) is added to the reaction mixture which is then heated to 40 C for 24 h. The reaction is cooled to room temperature and quenched with 60 mL MeOH until gas evolution ceases; 400 mL HCl is added and the reaction is refluxed for 3 h. The reaction is then cooled to room temperature and the water layer is washed with ethyl acetate. The water layer is then brought to pH 14 with 6 N NaOH and extracted with ethyl acetate. The combined organic extracts are dried (MgSO₄), filtered, and concentrated to crude product 5-bromo-2,3-dihydro-1H-isoindole, which is carried onto the next step without further manipulation.

### Synthesis of 6-Bromo-1,2,3,4-tetrahydro-isoquinoline

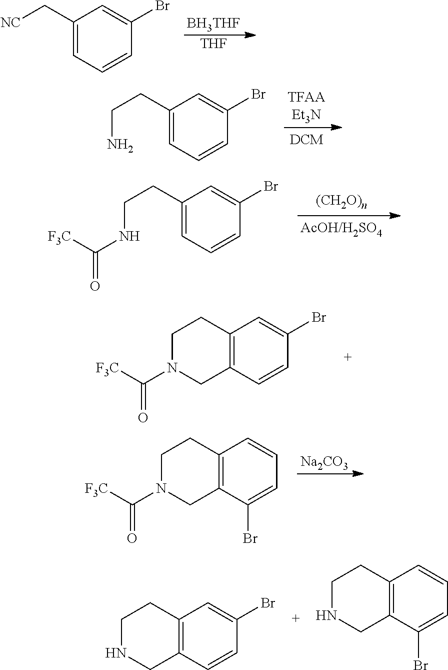

### 2-(3-Bromophenyl)ethylamine

To a solution of 3-bromophenyl acetonitrile (9.8 g, 50 mmol) in THF (20 mL) is added a solution of borane (0.1 M in THF, 150 mL) under a nitrogen atmosphere. The reaction mixture is refluxed overnight. It is cooled to room temperature and treated with 6 N HCl (150 mL). The resulting mixture is washed with ethyl acetate and ethyl acetate is discarded. The aqueous layer is basified with sodium carbonate and then extracted with ethyl acetate several times. The combined organic layer is washed with water, dried over sodium sulfate, and the solvent is removed under reduced pressure to obtain colorless viscous oil without further purification (7.3 g, 73%).

### Synthesis of N-[2-(3-Bromo-phenyl)-ethyl]-2,2,2-trifluoro-acetamide

To a solution of 2-(3-Bromophenyl)ethylamine (7.3 g, 36 mmol) and triethylamine (15 mL, 107 mmol) in dry dichloromethane (70 mL) is added trifluoroacetic anhydride drop-wise and the reaction mixture is stirred at room temperature for 2 h. The reaction mixture is diluted with dichloromethane, washed with water and brine, dried over sodium sulfate and concentrated. The crude product is purified by column chromatography (silica gel, 60-120 mesh) using 10% ethyl acetate in petroleum ether as eluent to obtain the product as light yellow solid (7.8 g, 72%).

### Synthesis of 1-(6-Bromo-3,4-dihydro-1H-isoquinolin-2-yl)-2,2,2-trifluoro-ethanone

To a solution of acetic acid and sulfuric acid are added N-[2-(3-Bromo-phenyl)-ethyl]-2,2,2-trifluoro-acetamide (7.8 g, 26 mmol) and paraformaldehyde (1.3 g, 43 mmol). The reaction mixture is stirred at room temperature for 16 h, poured into cold water and extracted with ethyl acetate. The combined organic layers are washed with sodium hydrogen carbonate solution, dried over sodium sulfate and evaporated. The crude product is purified by column chromatography (silica gel, 60-120 mesh) using 0-4% ethyl acetate in petroleum ether as eluent to obtain the product mixed with 1-(8-bromo-3,4-dihydro-1H-isoquinolin-2-yl)-2,2,2-trifluoro-ethanone as colorless liquid (3.1 g, 37%). MS m/z 308.0 (M+1)

### Synthesis of 6-Bromo-1,2,3,4-tetrahydro-isoquinoline

To a solution of methanol (20 mL) and saturated sodium carbonate solution (20 mL) are added a mixture of 1-(6-Bromo-3,4-dihydro-1H-isoquinolin-2-yl)-2,2,2-trifluoro-ethanone and 1-(8-bromo-3,4-dihydro-1H-isoquinolin-2-yl)-2,2,2-trifluoro-ethanone (3 g, 9.7 mmol). The reaction mixture is refluxed overnight, concentrated and the residue is extracted with dichloromethane. The combined organic layers are washed with water and brine, dried over sodium sulfate and the solvent is evaporated. The crude product is purified by column chromatography (silica gel, 230-400 mesh) using 0-2% methanol in chloroform as eluent to obtain 8-bromo-1,2,3,4-tetrahydro-isoquinoline as colorless viscous oil (first fraction, 0.45 g, 22%) and 6-Bromo-1,2,3,4-tetrahydro-isoquinoline as white solid (second fraction, 1.0 g, 48%). MS m/z 211.9 (M+1)

### Synthesis of 7-Bromo-1,2,3,4-tetrahydro-isoquinoline

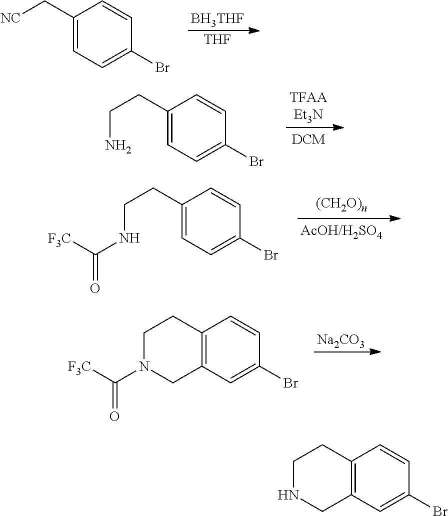

### Synthesis of 2-(4-Bromophenyl)ethylamine

To a solution of 4-bromophenyl acetonitrile (20 g, 102 mmol) in THF (20 mL) is added a solution of borane (1 M in THF, 300 mL) under a nitrogen atmosphere. The reaction mixture is stirred at 75 C overnight. It is cooled to room temperature and treated with 6 N HCl (500 mL) drop-wise and stirred further at room temperature for 5 h. The resulting mixture is washed with ethyl acetate and ethyl acetate is discarded. The aqueous layer is basified with sodium carbonate and then extracted with ethyl acetate several times. The combined organic layer is washed with water, dried (Na₂SO₄), and the solvent was removed under reduced pressure to obtain pale yellow oil without further purification (16 g, 78%).

### Synthesis of N-[2-(4-Bromo-phenyl)-ethyl]-2,2,2-trifluoro-acetamide

To a solution of 2-(4-Bromophenyl)ethylamine (16 g, 80 mmol) and triethylamine (34 mL, 244 mmol) in dry dichloromethane (150 mL) is added trifluoroacetic anhydride drop-wise and the reaction mixture is stirred at room temperature for 2 h. The reaction mixture is diluted with dichloromethane, washed with water and brine, dried (Na₂SO₄), and concentrated. The crude product is purified by column chromatography to obtain the product as off-white solid (15.7 g, 66%).

### Synthesis of 1-(7-Bromo-3,4-dihydro-1H-isoquinolin-2-yl)-2,2,2-trifluoro-ethanone

To a solution of 40% sulfuric acid in acetic acid (150 mL) are added N-[2-(4-Bromo-phenyl)-ethyl]-2,2,2-trifluoro-acetamide (15.5 g, 52.4 mmol) and paraformaldehyde (2.4 g, 80 mmol). The reaction mixture is stirred at room temperature for 18 h, poured into cold water and extracted with ethyl acetate. The combined organic layers are washed with sodium hydrogen carbonate solution, dried (Na₂SO₄), and evaporated under reduced pressure to give the product as pale yellow oily liquid which solidified on standing (14 g, 88%).

### Synthesis of 7-Bromo-1,2,3,4-tetrahydro-isoquinoline

To a solution of methanol (50 mL) and saturated sodium carbonate solution (50 mL) is added 1-(7-Bromo-3,4-dihydro-1H-isoquinolin-2-yl)-2,2,2-trifluoro-ethanone (14.3 g, 46.4 mmol). The reaction mixture is stirred at 60 C for 1 h, concentrated and the residue is extracted with ethyl acetate. The combined organic layers are washed with water and brine, dried (Na₂SO₄), and the solvent is evaporated. The crude product is purified by column chromatography to obtain product as pale yellow solid (8.4 g, 85%). MS m/z 213.8 (M+1)

### Synthesis of 7-Bromo-2,3,4,5-tetrahydro-1H-benzo[d]azepine and 7-Bromo-2,3,4,5-tetrahydro-1H-benzo[c]azepine

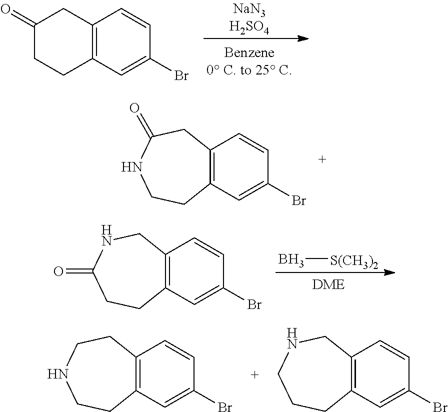

### 7-Bromo-1,3,4,5-tetrahydro-benzo[d]azepin-2-one and 7-Bromo-1,2,4,5-tetrahydro-benzo[c]azepin-3-one

To a cooled (0 C) solution of 6-Bromo-3,4-dihydro-1H-naphthalen-2-one (5.0 g, 22.2 mmol) in benzene is added sodium azide (5.77 g, 88.8 mmol) followed by drop-wise addition of concentrated sulfuric acid (10 mL). The ice bath is removed and the reaction mixture is stirred at room temperature overnight. The reaction mixture is diluted with ethyl acetate, washed with water (2×) and brine, dried (Na₂SO₄), and concentrated to provide the crude mixture of regioisomers (3.76 g, 71% combined yield). The mixture is taken together into the next step without further separation. LC-MS m/z 240 and 242 (M+1).

### 7-Bromo-2,3,4,5-tetrahydro-1H-benzo[d]azepine and 7-Bromo-2,3,4,5-tetrahydro-1H-benzo[c]azepine

To a solution of 7-Bromo-1,3,4,5-tetrahydro-benzo[d]azepin-2-one and 7-Bromo-1,2,4,5-tetrahydro-benzo[c]azepin-3-one mixture (3.76 g) in 1,2-Dimethoxyethane (25 mL) under nitrogen is added a solution of borane-dimethyl sulfide complex (10.0M, 3.13 mL, 31.3 mmol) and the reaction mixture is refluxed overnight. The mixture is quenched with MeOH to remove excess borane. The resulting mixture is concentrated in vacuo and dissolved in hydrogen chloride in methanol solution (HCl 1.25M in methanol). The mixture is stirred at room temperature for 20 minutes and concentrated in vacuo. The residual solid (hydrochloride salt) is taken to next step without further purification. The isomeric compounds can be separated in the next step via column chromomatography. LC-MS: m/z 226 and 228 (M+1).

### Preparation of Acrylic Acid Methyl Ester Heterocyclic Building Blocks

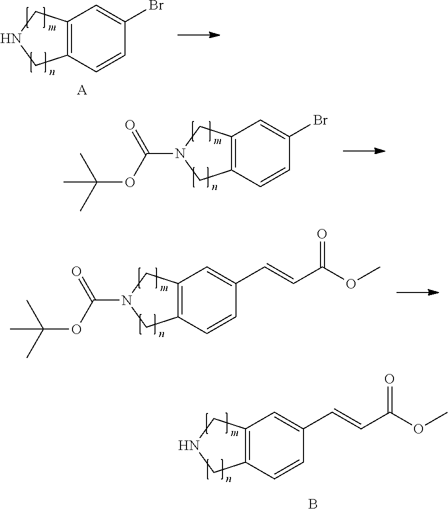

All aryl bromide heterocyclic building blocks can be converted to aryl bromide heterocyclic building blocks in a manner similar to the synthesis of (E)-3-(2,3-Dihydro-1H-isoindol-5-yl)-acrylic acid methyl ester or (E)-3-(1,2,3,4-Tetrahydro-isoquinolin-7-yl)-acrylic acid methyl ester.

### Synthesis of (E)-3-(2,3-Dihydro-1H-isoindol-5-yl)-acrylic acid methyl ester

### 5-Bromo-1,3-dihydro-isoindoline-2-carboxylic acid tert-butyl ester

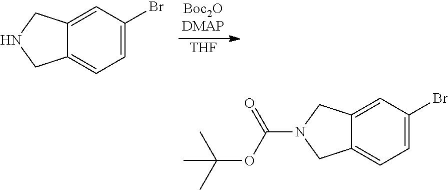

To a solution of 5-bromo-2,3-dihydro-1H-isoindole (8.75 g; 1.0 equiv) in THF (126 mL) are added di-tert-butyldicarbonate (10.8 g; 1.12 equiv) and 4-dimethylaminopyridine (5.4 g; 1.0 equiv) and the reaction is stirred at room temperature for 3 h. The reaction is quenched with saturated sodium hydrogen carbonate and extracted with ethyl acetate. The combined organic extracts are dried (MgSO₄), filtered, and concentrated to afford crude product, which is purified by silica gel chromatography to yield 7.0 g 5-bromo-1,3-dihydro-isoindole-2-carboxylic acid tert-butyl ester (23.49 mmol; 53% yield over two steps). ¹H NMR (400 MHz, CD₃OD): δ 7.39 (dd, J=4.0, 16.0 Hz, 1H), 7.26 (s, 1H), 7.12 (dd, J=20.0, 4.0 Hz), 4.67 (s, 1H), 4.62 (s, 2H), 4.59 (s, 1H), 1.51 (s, 9H).

### 5-(2-Methoxycarbonyl-vinyl)-1,3-dihydro-isoindoline-2-carboxylic acid tert-butyl ester

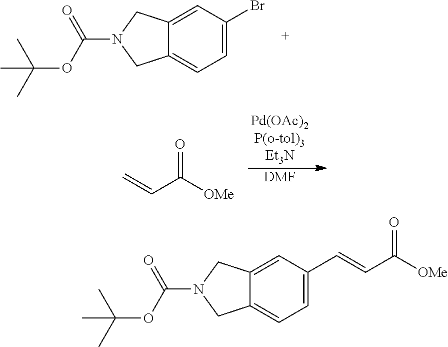

To a 100 mL 3-neck round bottom flask are added 5-bromo-1,3-dihydro-isoindole-2-carboxylic acid tert-butyl ester (13.42 mmol; 1.0 equiv), Pd(OAc)₂ (0.34 mmol; 0.025 equiv), and P(o-tol)₃ (0.67 mmol; 0.05 equiv) and the flask is evacuated and purged with N₂ three times. DMF (34 mL), methyl acrylate (14.76 mmol; 1.1 equiv), and Et₃N (67.1 mmol; 5.0 equiv) are added and the reaction mixture is heated to 130 C for 15 h. The reaction is cooled to room temperature, diluted with Et₂O (200 mL), and the organic layer is washed with 10% citric acid, saturated sodium hydrogen carbonate, and brine. The combined organic extracts are dried (MgSO₄), filtered, and concentrated to afford the crude product, which is then purified by silica gel chromatography to yield 5-(2-methoxycarbonyl-vinyl)-1,3-dihydro-isoindole-2-carboxylic acid tert-butyl ester as a yellow solid (1.7 g; 42% yield). ¹H NMR (400 MHz, CD₃OD): δ 7.68 (d, J=16 Hz, 1H), 7.53 (m, 2H), 7.32 (t, J=8.0 Hz, 1H), 6.52 (d, J=16 Hz, 1H), 4.64 (d, J=8 Hz, 4H), 3.78 (s, 3H), 1.52 (s, 9H). MS m/z 305.0 (M+1)⁺

### (E)-3-(2,3-Dihydro-1H-isoindol-5-yl)-acrylic acid methyl ester

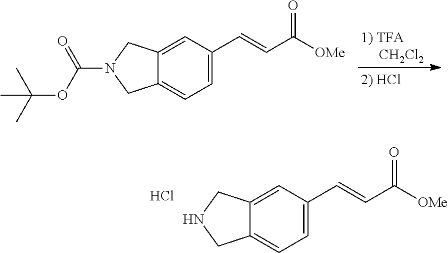

To a solution of 5-(2-methoxycarbonyl-vinyl)-1,3-dihydro-isoindole-2-carboxylic acid tert-butyl ester (988 mg; 3.26 mmol; 1.0 equiv) in anhydrous CH₂Cl₂ (10 mL) at 0 C is added trifluoroacetic acid drop-wise over 15 minutes. The reaction is allowed to warm slowly to room temperature over 1 h, before adding saturated sodium hydrogen carbonate. The water layer is extracted with CH₂Cl₂ and the combined organic extracts are dried (MgSO₄), filtered, and concentrated to dryness. The resulting yellow oil is taken up in anhydrous MeOH at room temperature and excess HCl in dioxane (4.0 M) is added. The solvent is removed in vacuo and the resulting gray solid is triturated with Et₂O. The precipitate is collected via filtration and dried to give (E)-3-(2,3-dihydro-1H-isoindol-5-yl)-acrylic acid methyl ester (853 mg). ¹H NMR (400 MHz, CD₃OD): δ 7.70 (d, J=16.0 Hz, 1H), 7.56 (s, 1H), 7.48 (d, J=8.0 Hz, 1H), 7.33 (d, J=8.0 Hz, 1H), 6.52 (d, J=16.0 Hz, 1H), 4.23 (s, 4H) 3.78 (s, 3H). MS m/z 204.0 (M+1)⁺

### Synthesis of (E)-3-(1,2,3,4-Tetrahydro-isoquinolin-7-yl)-acrylic acid methyl ester

### 7-((E)-2-Methoxycarbonyl-vinyl)-3,4-dihydro-1H-isoquinoline-2-carboxylic acid tert-butyl

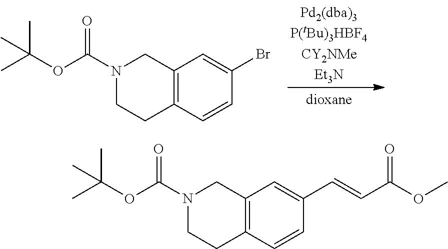

7-Bromo-3,4-dihydro-1H-isoquinoline-2-carboxylic acid tert-butyl ester (300 mg, 095 mmol), Pd₂(dba)₃ (8.7 mg, 0.0095 mmol) and P(tBu)₃HBF₄ (11.0 mg, 0.0379 mmol) are placed in a microwave reaction vial. The system is vacuumed and then filled with N₂ for several cycles. Dioxane (10 ml) is added in, followed by the addition of acrylic acid methyl ester (167 mg, 1.90 mmol) and Cy₂NMe (242 uL, 1.14 mmol). The vial is placed in microwave and heated at 100 C for 1 h. The mixture is diluted with EtOAc, washed with water, brine and dried. After concentrated, the crude material is purified by flash chromatography to afford 280 mg (92.7%) of the title compound.

### (E)-3-(1,2,3,4-Tetrahydro-isoquinolin-7-yl)-acrylic acid methyl ester

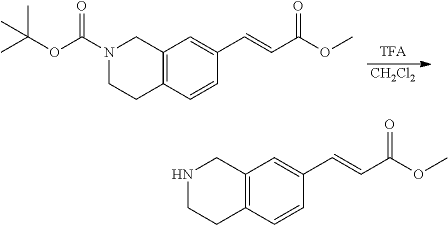

To the solution of 7-((E)-2-Methoxycarbonyl-vinyl)-3,4-dihydro-1H-isoquinoline-2-carboxylic acid tert-butyl ester (280 mg, 0.882 mmol) in dichloromethane (10 ml) is added TFA (4 ml). The reaction is stirred at room temperature for about 3 h. The solvent is removed to afford 250 mg of the title compound.

### Synthesis of 7-Bromo-1,2,4,5-tetrahydro-benzo[d]azepine-3-carboxylic acid tert-butyl ester and 7-Bromo-1,3,4,5-tetrahydro-benzo[c]azepine-2-carboxylic acid tert-butyl ester

### 7-Bromo-1,2,4,5-tetrahydro-benzo[d]azepine-3-carboxylic acid tert-butyl ester and 7-Bromo-1,3,4,5-tetrahydro-benzo[c]azepine-2-carboxylic acid tert-butyl ester

To a suspension of 7-Bromo-2,3,4,5-tetrahydro-1H-benzo[d]azepine and 7-Bromo-2,3,4,5-tetrahydro-1H-benzo[c]azepine mixture in anhydrous CH₂Cl₂ (40 mL) is added diisopropylethylamine (5.7 mL, 33 mmol), followed by di-tert-butyldicarbonate (3.6 g, 16.5 mmol) under nitrogen and stirred at room temperature for 2 h. The reaction mixture is diluted with ethyl acetate, washed with 1 N hydrochloric acid (2×), brine, dried (Na₂SO₄) and concentrated in vacuo. The residue was purified via silica gel chromatography to give the title compounds (3.37 g total, yield 45%, overall 3 steps).

7-Bromo-1,2,4,5-tetrahydro-benzo[d]azepine-3-carboxylic acid tert-butyl ester ¹H NMR (400 MHz, CDCl₃): δ 7.28 (s, 1H), 7.27 (d, J=8 Hz, 1H), 7.00 (d, J=8 Hz, 1H), 3.56 (t, J=4 Hz, 2H), 3.55 (t, J=4 Hz, 2H), 2.88-2.86 (m, 4H), 1.50 (s, 9H). LCMS: m/z 270/272 (M+1-tBu).

7-Bromo-1,3,4,5-tetrahydro-benzo[c]azepine-2-carboxylic acid tert-butyl ester ¹H NMR (400 MHz, CDCl₃): δ 7.31-27 (m, 2H), 7.11-7.01 (m, 1H), 4.35 (br s, 2H), 3.89 (br s, 2H), 2.94-2.91 (m, 2H), 1.79-1.77 (m, 2H), 1.51 (s, 9H). LC-MS: m/z 270/272 (M+1-tBu).

### 7-((E)-2-Methoxycarbonyl-vinyl)-1,2,4,5-tetrahydro-benzo[d]azepine-3-carboxylic acid tert-butyl ester

To a flame-dried microwave vial are added Pd₂(dba)₃ (14 mg, 0.015 mmol) and P(t-Bu)₃.HBF₄ (17 mg, 0.16 mmol), then the vial is evacuated and purged with N₂ three times. After that, a solution of 7-bromo-1,2,4,5-tetrahydro-benzo[d]azepine-3-carboxylic acid tert-butyl ester (510 mg, 1.56 mmol, 1.0 equiv) in dioxane (1.5 mL) and N-methyl-dicyclohexylamine (400 μL, 1.9 mmol) are added sequentially. The reaction mixture is stirred at room temperature for 0.5 h. Then methyl acrylate (280 μL, 3.12 mmol) is added under N₂ and the reaction vial is put in microwave reactor for 0.5 h at 100 C. The resulting mixture is diluted with ethyl acetate, filtered through Celite, concentrated and purified via silica gel chromatography to give the title compound (370 mg, 72% yield). ¹H NMR (400 MHz, CDCl₃): δ 7.67 (d, J=16 Hz, 1H), 7.32-7.28 (m, 2H), 7.15 (d, J=8 Hz, 1H), 6.42 (d, J=16 Hz, 1H), 3.82 (s, 3H), 3.59-3.56 (m, 4H), 2.94-2.92 (m, 4H), 1.50 (s, 9H). LCMS: m/z 276 (M+1-tBu), 232 (M+1-Boc).

### Synthesis of 7-((E)-2-Methoxycarbonyl-vinyl)-1,3,4,5-tetrahydro-benzo[c]azepine-2-carboxylic acid tert-butyl ester

The title compound is prepared from 7-Bromo-1,3,4,5-tetrahydro-benzo[c]azepine-2-carboxylic acid tert-butyl ester as described in preparation of 7-((E)-2-Methoxycarbonyl-vinyl)-1,2,4,5-tetrahydro-benzo[d]azepine-3-carboxylic acid tert-butyl ester. (yield 84%). ¹H NMR (400 MHz, CDCl₃): δ 6.77 (d, J=16 Hz, 1H), 6.43-6.36 (m, 2H), 5.54 (d, J=16 Hz, 1H), 3.51 (br s, 2H), 2.92 (s, 3H), 2.81 (br s, 2H), 2.07 (br s, 2H), 0.90 (br s, 2H), 0.51 (s, 9H). LCMS: m/z 276 (M+1-tBu), 232 (M+1-Boc).

### Synthesis of (E)-3-(2,3,4,5-Tetrahydro-1H-benzo[d]azepin-7-yl)-acrylic acid methyl ester

A solution of 7-((E)-2-Methoxycarbonyl-vinyl)-1,2,4,5-tetrahydro-benzo[d]azepine-3-carboxylic acid tert-butyl ester (370 mg, 1.11 mmol) in CH₂Cl₂ (2 mL) is cooled to −20 C and TFA (2 mL) is added to this solution. Then the solution is allowed to warm to room temperature and stirred for 1 hour, and concentrated in vacuo. The residue is diluted with CH₂Cl₂, washed with saturated sodium hydrogen carbonate solution, brine; dried (Na₂SO₄) and concentrated to give the title compound (200 mg, yield 77%). LCMS: m/z 232 (M+1)

### Synthesis of (E)-3-(2,3,4,5-Tetrahydro-1H-benzo[c]azepin-7-yl)-acrylic acid methyl ester

The title compound is prepared from 7-((E)-2-Methoxycarbonyl-vinyl)-1,3,4,5-tetrahydro-benzo[c]azepine-2-carboxylic acid tert-butyl ester as described in the preparation of (E)-3-(2,3,4,5-Tetrahydro-1H-benzo[d]azepin-7-yl)-acrylic acid methyl ester. (yield 93%). LCMS: m/z 232 (M+1).

### Preparation of N-Substituted-Heterocyclic Compounds

A typical procedure for reductive amination using sodium triacetoxyborohydride:

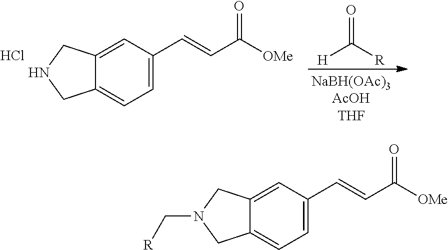

To a solution of (E)-3-(2,3-dihydro-1H-isoindol-5-yl)-acrylic acid methyl ester (1.0 equiv) in THF is added the aldehyde (1.0 equiv), and the reaction is stirred at room temperature for 1 h. Sodium triacetoxyborohydride (1.4 equiv) and excess AcOH (1 mL) is added and the reaction stirred at room temperature for 6 h. The reaction is diluted with EtOAc. The combined organic extracts are dried (MgSO₄), filtered, and concentrated. The crude product is purified by silica gel chromatography.

A typical procedure for reductive amination using titanium tetrachloride:

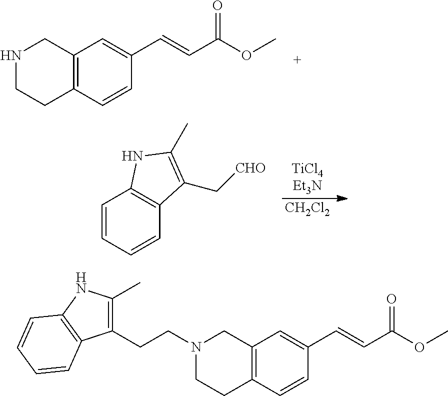

To a solution of (E)-3-(1,2,3,4-Tetrahydro-isoquinolin-7-yl)-acrylic acid methyl ester (249 mg, 1.15 mmol) in dichloromethane (10 ml) is added (2-Methyl-1H-indol-3-yl)-acetaldehyde (200 mg, 1.15 mmol), triethylamine (479 uL, 3.44 mmol). TiCl₄ (560 uL, 1 M in dichloromethane) is added dropwise. The reaction is monitored by TLC. Once the starting material is consumed, the reaction is quenched by NaCNBH₄(228 mg, 3.45 mmol) in MeOH (3 ml) and stirred for 12 h. The reaction is basified to pH 13 with 5 N NaOH, extracted with EtOAc, dried, concentrated, and purified by chromatography to afford 100 mg (yield: 23.3%) of (E)-3-{2-[2-(2-Methyl-1H-indol-3-yl)-ethyl]-1,2,3,4-tetrahydro-isoquinolin-7-yl}-acrylic acid methyl ester.
A typical procedure for sulfonamide formation:

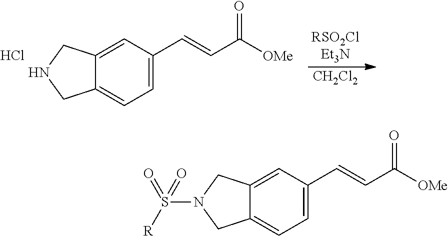

To a solution of (E)-3-(2,3-dihydro-1H-isoindol-5-yl)-acrylic acid methyl ester (1.0 equiv) in CH₂Cl₂ is added Et₃N (excess), followed by the sulfonyl chloride (1.0 equiv) and the reaction is stirred at room temperature for 5 h. The reaction mixture is washed with 10% citric acid, saturated sodium hydrogen carbonate, and brine. The combined organic extracts are dried (MgSO₄), filtered, and concentrated to yield the crude product which is purified by silica gel chromatography.
A typical procedure for acylation:

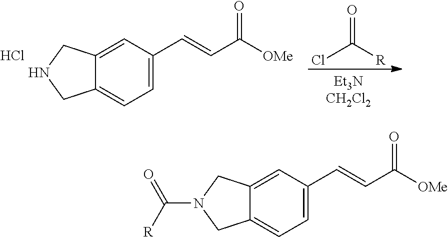

To a solution of (E)-3-(2,3-dihydro-1H-isoindol-5-yl)-acrylic acid methyl ester (1.0 equiv) in CH₂Cl₂ is added Et₃N (excess), followed by the acyl chloride (1.0 equiv) and the reaction is stirred at room temperature for 5 h. The reaction is diluted with saturated sodium hydrogen carbonate and the water layer is extracted with CH₂Cl₂. The combined organic extracts are dried (MgSO₄), filtered, and concentrated. The crude product is purified by silica gel chromatography.

A typical procedure for the conversion of methyl esters to N-hydroxy amides:

### Example 39

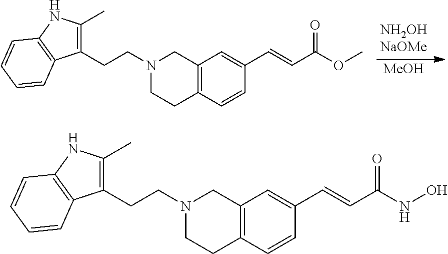

To a cooled (0 C) solution of (E)-3-{2-[2-(2-Methyl-1H-indol-3-yl)-ethyl]-1,2,3,4-tetrahydro-isoquinolin-7-yl}-acrylic acid methyl ester (100 mg, 0.264 mmol) in MeOH (2 mL) are added hydroxylamine (175 uL, 50% in H₂O, 2.65 mmol) and NaOMe (290 uL, 25% in MeOH, 1.34 mmol). The mixture is stirred at 0 C for 1.5 h, neutralized by 1 N HCl. Some solid crash out and form gum like stuff. The mixture is taken up in MeOH and purified by prep. HPLC to afford 15 mg (yield: 15%) of (E)-N-Hydroxy-3-{2-[2-(2-methyl-1H-indol-3-yl)-ethyl]-1,2,3,4-tetrahydro-isoquinolin-7-yl}-acrylamide.

### Spectroscopic data for representative N-substituted 2,3 dihidro-isoindoline compounds

### Example 1

### (E)-3-(2-Benzyl-2,3-dihydro-1H-isoindol-5-yl)-N-hydroxy-acrylamide

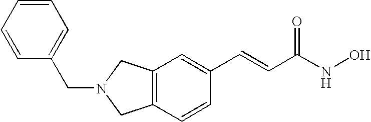

¹H NMR (400 MHz, CD₃OD): δ 7.56 (d, J=16 Hz, 1H), 7.39 (m, 8H), 6.45 (d, J=16 Hz, 1H), 4.21 (s, 4H), 4.18 (s, 2H), MS m/z 295.0 (M+1)⁺

### Example 8

### (E)-N-Hydroxy-3-[2-(toluene-4-sulfonyl)-2,3-dihydro-1H-isoindol-5-yl]-acrylamide

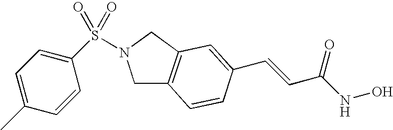

¹H NMR (400 MHz, CD₃OD): δ 7.77 (d, J=8.0 Hz, 2H). 7.51 (d, J=16.0 Hz, 1H), 7.41 (m, 4H,), 7.23 (d, J=8.0 Hz, 1H), 6.41 (d, J=16.0 Hz, 1H), 4.59 (s, 4H), 2.39 (s, 3H). MS m/z 359.0 (M+1)⁺

### Example 25

### (E)-N-Hydroxy-3-{2-[2-(2-methyl-1H-indol-3-yl)-acetyl]-2,3-dihydro-1H-isoindol-5-yl}-acrylamide

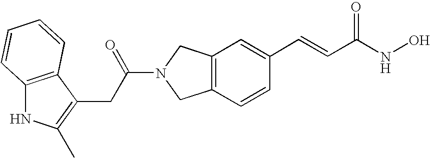

¹H NMR (400 MHz, CD₃OD): δ 7.51 (m, 4H), 7.29 (m, 2H), 6.99 (tt, J=8.0 Hz, 2H), 6.45 (dd, J=12.0 Hz, 1H), 4.86 (s, 2H), 4.77 (s, 2H), 3.86 (s, 2H), 2.43 (s, 3H), MS m/z 376.1 (M+1)⁺

### Example 80

### Synthesis of (E)-N-Hydroxy-3-{2-[2-(2-methyl-1H-indol-3-yl)-ethyl]-2,3,4,5-tetrahydro-1H-benzo[c]azepin-7-yl}-acrylamide

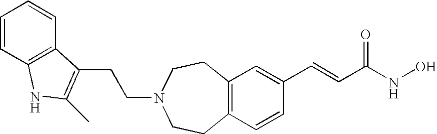

¹H NMR (400 MHz, DMSO): δ 10.58 (s, 1H), 7.34-7.18 (m, 5H), 7.11 (d, J=8 Hz, 1H), 6.88-6.77 (m, 2H), 6.36 (d, J=16 Hz, 1H), 3.92 (br s, 2H), 3.09 (br s, 2H), 2.83-2.81 (m, 2H), 2.70 (t, J=8 Hz, 2H), 2,36-2,32 (m, 2H), 2.18 (s, 3H), 1.57 (br s, 2H). ¹³C NMR (400 MHz, DMSO): δ 162.78, 143.31, 138.12, 135.07, 133.53, 131.55, 130.39, 128.16, 127.56, 124.95, 119.74, 118.41, 117392, 117.253, 110.27, 107.95, 58.41, 57.72, 53.01, 35.33, 24.15, 22.02, 11.19; HRMS calcd for C₂₄H₂₇N₃O₂.H⁺ (M+H⁺) 390.2182. Found 390.2173.

### Example 41

### (E)-N-Hydroxy-3-{3-[2-(2-methyl-1H-indol-3-yl)-ethyl]-2,3,4,5-tetrahydro-1H-enzo[d]azepin-7-yl}-acrylamide

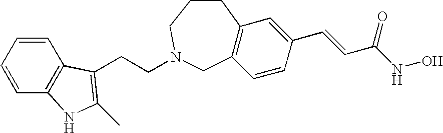

¹H NMR (400 MHz, DMSO): δ 10.14 (s, 1H), 7.48-7.35 (m, 4H), 7.27 (d, J=8 Hz, 1H), 7.22 (d, J=8 Hz, 1H), 7.04-6.95 (m, 2H), 6.47 (d, J=16 Hz, 1H), 2.96-2.84 (m, 6H), 2.77-2.66 (m, 6H), 2.38 (s, 3H). ¹³C NMR (400 MHz, DMSO): δ 162.85, 143.77, 142.58, 138.26, 135.11, 132.65, 131.62, 129.36, 128.19, 127.68, 125.40, 119.79, 118.01, 117.23, 110.31, 108.08, 59.35, 54.77, 35.75, 35.69, 21.17, 11.21; HRMS calcd for C₂₄H₂₇N₃O₂.H⁺ (M+H⁺) 390.2182. Found 390.2183.

### Biological Activity

### Materials and Methods

### Immunoprecipitation of Deacetylase from Stable Cell Lines and Elution

Conventional methods are used to express deacetylase enzymes and purify them from lysed cells. The following example describes an exemplary procedure, however equivalent procedures are within the scope of the invention.

The cell line used is a derivative of 293 cells overexpressing a fusion of the gene encoding each deacetylase protein with a nucleotide sequence encoding the Flag marker.

Cells are grown in Optimem, 2% Fetal Calf Serum, Pen/Strep. For enzyme preparation, Lysis buffer (IPLS) is 50 mM Tris-HCl, pH 7.5, 120 mM NaCl, 0.5 mM EDTA and 0.5% Nonidet P-40, to which is added one tablet of Protease inhibitors (Roche 11836170001) per 10 ml buffer. Other buffers are IPHS, which is IPLS containing 1 M NaCl; TBS (Sigma #T5912) dilute 10× stock to 1× with dH₂O; HD buffer: 10 mM Tris pH 8.0 (1M Stock) 10 mM NaCl (5M Stock), 10% glycerol, and for dialysis: 400 μM PMSF is added (for 2 L: use 8 ml 100 mM Stock). Protease inhibitors (Complete mini, Boehringer Mannheim), 1 tablet/10 mL are added to all buffers but not used in buffers for enzyme assays.

Cells are harvested without using trypsin, and most cells are obtained easily in PBS, with gentle striking or agitation of flasks if necessary. More adherent cells are scraped in PBS. Cells are grown in 500 cm² trays, from which about half of the media is aspirated (50 ml total), then cells are scraped in the rest of the media and transferred to a centrifuge tube. Trays are washed with 25 ml cold PBS, scraped again to collect additional cells, and centrifuged at 1500 rpm at 4° C. for 5 min. Cells are washed at least 3 times in PBS to remove growth media, pelleting cells after each wash by centrifugation at 1500 rpm for 5 minutes. After washing, PBS is removed and the resulting cell pellet frozen at −80° C. for storage prior to purification.

For purification, cells are resuspended in lysis buffer, 12 ml of IPLS for cells collected from 10 500 cm trays. Cells are lysed at 4° C. for 3 hrs with rocking, and debris is removed by centrifugation for 20 min at 17,000 rpm in 30 ml centrifuge tubes. If supernatant is not clear afterward, centrifugation of the supernatant is repeated. Protein concentration of the whole cell lysate is determined (generally in the range of about 2-5 mg/ml).

For immunoprecipitation per mg of protein, 15 μL of anti-Flag M2-Agarose Affinity beads (Sigma #A2220) is used. Beads are prepared by washing 3 times with 10× bead volume of PBS and 1 time with IPLS, with centrifugation of the washes at 1500 rpm for 5 min. Whole cell lysate is incubated with the Ab-beads overnight at 4° C. Then beads are centrifuged and washed in 5× volume of the following buffers: three times in IPLS (30 sec at 4° C., spin at 1500 RPM for 5 min); three times in IPHS; and three times in TBS buffer. After each centrifugation, the supernatant is aspirated, leaving the pellet as dry as possible but avoiding sucking up any of the beads.

To elute the enzyme, beads are resuspended in 5× bead volume of TBS with protease inhibitor (Roche 11836170001) 1 tablet/10 mL. Enzyme is eluted with 400 μg/mL Flag peptide (Sigma #F-3290) for 3 hrs at 4° C. on rotator. Then beads are centrifuged, and the supernatant is transferred to a new tube to which is added 1/10 volume of glycerol. The supernatant is transferred to a dialysis cassette (Pierce #66410) using a 3 cc syringe and 18 G needle, and is dialyze sup in 2 L HD buffer for 2 hrs at 4° C. (1 L/hour). The resulting purified deacetylase is divided into aliquots (300 μL/tube), is snap frozen in dry ice bath, and is stored at −80° C.

### Deacetylase Fluorescence Assay

For assay of deacetylase an assay based on Deacetylase Fluorescent Activity Assay/Drug Discovery Kit (BioMol # AK500) is used, however any equivalent deacetylase assay is within the scope of the invention.

The Fluorescent Assay Buffer (FAB) contains: 25 mM Tris-HCl, pH 8.0, 137 mM NaCl, 2.7 mM KCl and 1 mM MgCl₂. To prepare 20× Developer: 27 mg/mL Trypsin (Sigma #T-8003) is dissolved in Fluorescent Assay Buffer, and is divided into aliquots and stored at −80 C (250 μL/96-well plate). For use, the Developer is diluted to 1× and added 10 μL/mL 0.2 mM TSA

Final assay concentrations are: up to 15 μL deacetylase isoform enzyme, 25 μL of substrate (25 uM of rhodamine, 50 uM Fluor de lys substrate, BIOMOL, Plymouth Meeting PA available as kit AK-500), and ±10 μL inhibitor diluted in FAB. The final reaction volume of 50 μL is obtained by adding FAB.

All reaction components are prepared in Fluorescent Assay Buffer; enzyme and diluted inhibitors (total volume is 25 μL) are added to clear bottom 96-well ISOPLATE (Wallac #1450-514). The reactions are initiated by adding 25 μL of 100 μM substrate. Negative control wells contain buffer and substrate only or with potent levels of LAQ824 inhibitor.

Enzyme reactions with DMSO are used as positive controls.

The reaction is run for 1-2 hours at 37 C, and reactions are stopped with 50 μL/well of 1× developer containing TSA. Reactions are developed at room temperature for 10 min, and are read with a pre-warmed lamp of Cytofluor Fluorescence Reader. For Fluor de Lys: plates are read at Excitation 360 nm, Emission 460 nm, Gain 65. For Rhodamine: plates are read at Excitation 485 nm, Emission 530 nm, Gain 60.

### p21 Promoter Luciferase Assay Using Stably Transfected p21-Luc in H1299 Cells

### Reagents and General Conditions

The cell lines used are derived from H1299 (p21-luc). The growth media used is RPMI 1640, 10% FBS, 1% Pen/Strep and the selection media added is 500 μg/mL Geneticin (Gibco). The buffer used is 5× cell culture lysis buffer (Promega #E1531), stored at −20 C and the Luciferase assay reagent (Promega #E1483) is stored at −70° C. The results of the assay are analyzed using Wallac Software.

To assay Luciferase, the cell culture medium is removed after one day of growth and the flasks are washed once with PBS. The cells are trypsinized in 20 mL of media and the trypsin is neutralized. The cells are counted (0.5-1 mL) on a Vi-Cell XR cell viability analyzer.

Cells are then diluted to a concentration of approximately 5000 cells/200 μL, and 190 μL samples are aliquoted into each well of a Costar white 96-well TC treated white bottom plate with lid (Costar #3917). Plates are then incubated overnight at 37 C.

After a further day, a sample of the compound is added to the wells for assay.

After a further day, the cells are lysed and the luciferase activity of the lysed cells is measured. Each well is washed twice with PBS and 20 μL/well of 1× cell culture lysis buffer (dilute 5× to 1× in distilled water) is added to each well. The microtiter plates are then shaken on a microtiter plate shaker for 20 minutes at room temperate at a speed setting of 5-6. After removal from the shaker, 100 μL of Luciferase Reagent is added to each well. Each microtiter plate is then read on Wallac Envision instrument.

Results from this experiment for compounds 1-118 are shown in Table A.

### Screening Inhibitory Activity of Examples 1-118

The general procedure to determine the IC50 of the compound using an in vitro cell based assay is as follows: cells are seeded into wells of 96-well plates as described above, and are incubated for growth for 24 hours, after which an aliquot of the compound is added at a variety of dilutions to the cells in each well. After further incubation of 72 hours, plates are read.

In general, serial dilutions of the compound are made in cell growth media, and 10 ul samples of diluations of the compound are added to the cells, in triplicate (3 rows). Plates are incubated at 37° C. for 72 hours. For determination of activity, CellTiter 96® AQueous One Solution Reagent (Promega), stored frozen, is thawed, protected from light. A sample of 10 μl of CellTiter 96® AQueous One Solution Reagent is added into wells of the 96-well assay plate. Plates are incubated for 3 hours at 37° C. in a humidified, 5% CO₂ atmosphere, and the absorbance at 490 nm is recorded using a 96-well plate reader.

Results from this experiment for compounds 1-118 are shown in Table A.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method of treating a deacetylase-associated disorder comprising administering to a subject in need thereof a pharmaceutically acceptable amount of a heterocyclic compound such that the deacetylase-associated disorder is treated.

2. The method of claim 1, wherein in the heterocyclic compound is an isoindoline derivative, a tetrahydro-isoquinoline derivative, or a tetrahydro-benzazepine derivative.

3. The method of claim 1, wherein the heterocyclic compound is a compound of the Formula I: 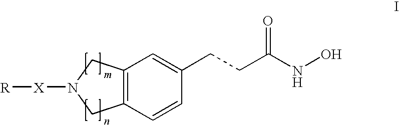
wherein
the dashed line indicates a single or double bond,
n and m are each, independently, 1, 2, or 3, and the sum of n and m is 2, 3 or 4;
wherein X is (CH₂)ⱼ wherein each CH₂ may be independently replaced one or more times with C(O), S(O)₂, S(O), O, or NR², wherein R² is selected from the group consisting of H, alkyl, aryl, heterocycle, C₁₋₄-alkyl, and C₃₋₆-cycloalkyl;
j is an integer between 0 and 6.
R is selected from the group consisting of C₁₋₄-alkyl, C₃₋₆-cycloalkyl and aryl, wherein cycloalkyl and aryl may be further independently substituted one or more times with aryl, heterocycle, C₁₋₄-alkyl, C₁₋₄-alkoxy, halogen, amino, nitro, cyano, pyrrolidinyl or CF₃.

4. The method of claim 3, wherein the compound is selected from the group consisting of (E)-3-(2-Benzyl-2,3-dihydro-1H-isoindol-5-yl)-N-hydroxy-acrylamide, 5-((E)-2-Hydroxycarbamoyl-vinyl)-1,3-dihydro-isoindole-2-carboxylic acid tert-butyl ester, (E)-3-[2-(2,2-Dimethyl-propionyl)-2,3-dihydro-1H-isoindol-5-yl]-N-hydroxy-acrylamide, (E)-N-Hydroxy-3-(2-methanesulfonyl-2,3-dihydro-1H-isoindol-5-yl)-acryl amide, (E)-3-(2-Benzenesulfonyl-2,3-dihydro-1H-isoindol-5-yl)-N-hydroxy-acrylamide, (E)-N-Hydroxy-3-[2-(pyridine-4-sulfonyl)-2,3-dihydro-1H-isoindol-5-yl]-acrylamide, (E)-N-Hydroxy-3-[2-(pyridine-3-sulfonyl)-2,3-dihydro-1H-isoindol-5-yl]-acrylamide, (E)-N-Hydroxy-3-[2-(toluene-4-sulfonyl)-2,3-dihydro-1H-isoindol-5-yl]-acrylamide, (E)-N-Hydroxy-3-[2-(4-methoxy-benzenesulfonyl)-2,3-dihydro-1H-isoindol-5-yl]-acrylamide, (E)-N-Hydroxy-3-[2-(4-trifluoromethyl-benzenesulfonyl)-2,3-dihydro-1H-isoindol-5-yl]-acrylamide, (E)-N-Hydroxy-3-[2-(toluene-3-sulfonyl)-2,3-dihydro-1H-isoindol-5-yl]-acrylamide, (E)-N-Hydroxy-3-[2-(3-trifluoromethyl-benzenesulfonyl)-2,3-dihydro-1H-isoindol-5-yl]-acrylamide, (E)-N-Hydroxy-3-[2-(3-methoxy-benzenesulfonyl)-2,3-dihydro-1H-isoindol-5-yl]-acrylamide, (E)-N-Hydroxy-3-[2-(toluene-2-sulfonyl)-2,3-dihydro-1H-isoindol-5-yl]-acrylamide, (E)-N-Hydroxy-3-[2-(1-methyl-1H-imidazole-4-sulfonyl)-2,3-dihydro-1H-isoindol-5-yl]-acrylamide, (E)-N-Hydroxy-3-[2-(3-trifluoromethyl-benzoyl)-2,3-dihydro-1H-isoindol-5-yl]-acrylamide, (E)-N-Hydroxy-3-[2-(pyridine-3-carbonyl)-2,3-dihydro-1H-isoindol-5-yl]-acrylamide, (E)-N-Hydroxy-3-[2-(thiophene-2-carbonyl)-2,3-dihydro-1H-isoindol-5-yl]-acrylamide, (E)-3-[2-(Butane-1-sulfonyl)-2,3-dihydro-1H-isoindol-5-yl]-N-hydroxy-acrylamide, (E)-N-Hydroxy-3-(2-phenylmethanesulfonyl-2,3-dihydro-1H-isoindol-5-yl)-acrylamide, (E)-3-(2-Cyclohexanecarbonyl-2,3-dihydro-1H-isoindol-5-yl)-N-hydroxy-acrylamide, (E)-3-(2-Cyclopentanecarbonyl-2,3-dihydro-1H-isoindol-5-yl)-N-hydroxy-acrylamide, (E)-3-(2-Cyclopropanecarbonyl-2,3-dihydro-1H-isoindol-5-yl)-N-hydroxy-acrylamide, (E)-3-(2-Cyclopropanesulfonyl-2,3-dihydro-1H-isoindol-5-yl)-N-hydroxy-acrylamide, (E)-N-Hydroxy-3-{2-[2-(2-methyl-1H-indol-3-yl)-acetyl]-2,3-dihydro-1H-isoindol-5-yl}-acrylamide, (E)-N-Hydroxy-3-{2-[2-(1H-indol-3-yl)-ethyl]-2,3-dihydro-1H-isoindol-5-yl}-acrylamide, (E)-N-Hydroxy-3-[2-(2-pyrazolo[1,5-a]pyridin-3-yl-ethyl)-2,3-dihydro-1H-isoindol-5-yl]-acrylamide, (E)-N-Hydroxy-3-{2-[2-(2-methyl-1H-indol-3-yl)-ethyl]-2,3-dihydro-1H-isoindol-5-yl}-acrylamide, (E)-N-Hydroxy-3-{2-[2-(2-methyl-pyrazolo[1,5-a]pyridin-3-yl)-ethyl]-2,3-dihydro-1H-isoindol-5-yl}-acrylamide, (E)-N-Hydroxy-3-{2-[2-(2-methyl-1H-pyrrolo[2,3-b]pyridin-3-yl)-ethyl]-2,3-dihydro-1H-isoindol-5-yl}-acrylamide, (E)-3-{2-[2-(2-Ethyl-pyrazolo[1,5-a]pyridin-3-yl)-ethyl]-2,3-dihydro-1H-isoindol-5-yl}-N-hydroxy-acrylamide, (E)-3-{2-[2-(2-tert-Butyl-1H-indol-3-yl)-ethyl]-2,3-dihydro-1H-isoindol-5-yl}-N-hydroxy-acrylamide, (E)-N-Hydroxy-3-{2-[2-(2-methyl-1H-pyrrolo[2,3-b]pyridin-3-yl)-ethyl]-2,3-dihydro-1H-isoindol-5-yl}-acrylamide, (E)-3-{2-[2-(2-tert-Butyl-1H-pyrrolo[2,3-b]pyridin-3-yl)-ethyl]-2,3-dihydro-1H-isoindol-5-yl}-N-hydroxy-acrylamide, (E)-N-Hydroxy-3-(2-phenethyl-2,3-dihydro-1H-isoindol-5-yl)-acrylamide, (E) —N-Hydroxy-3-{2-[2-(3-methyl-5-phenyl-isoxazol-4-yl)-ethyl]-2,3-dihydro-1H-isoindol-5-yl}-acrylamide, (E)-3-[2-(2-Cyclohexyl-ethyl)-2,3-dihydro-1H-isoindol-5-yl]-N-hydroxy-acrylamide, (E)-N-Hydroxy-3-{2-[2-(1H-indol-3-yl)-ethyl]-1,2,3,4-tetrahydro-isoquinolin-7-yl}-acrylamide, N-Hydroxy-3-{2-[2-(1H-indol-3-yl)-ethyl]-1,2,3,4-tetrahydro-isoquinolin-7-yl}-propionamide, (E)-N-Hydroxy-3-[2-(2-pyrazolo[1,5-a]pyridin-3-yl-ethyl)-1,2,3,4-tetrahydro-isoquinolin-7-yl]-acrylamide, (E)-N-Hydroxy-3-{2-[2-(3-methyl-5-phenyl-isoxazol-4-yl)-ethyl]-1,2,3,4-tetrahydro-isoquinolin-7-yl}-acrylamide, (E)-N-Hydroxy-3-{2-[2-(2-methyl-1H-indol-3-yl)-ethyl]-1,2,3,4-tetrahydro-isoquinolin-7-yl}-acrylamide, (E)-N-Hydroxy-3-{2-[2-(2-methyl-1H-pyrrolo[2,3-b]pyridin-3-yl)-ethyl]-1,2,3,4-tetrahydro-isoquinolin-7-yl}-acrylamide, (E)-N-Hydroxy-3-{2-[2-(2-methyl-pyrazolo[1,5-a]pyridin-3-yl)-ethyl]-1,2,3,4-tetrahydro-isoquinolin-7-yl}-acrylamide, (E)-3-{2-[2-(2-Ethyl-pyrazolo[1,5-a]pyridin-3-yl)-ethyl]-1,2,3,4-tetrahydro-isoquinolin-7-yl}-N-hydroxy-acrylamide, (E)-3-{2-[2-(2-tert-Butyl-1H-indol-3-yl)-ethyl]-1,2,3,4-tetrahydro-isoquinolin-7-yl}-N-hydroxy-acrylamide, (E)-3-{2-[2-(2-tert-Butyl-1H-pyrrolo[2,3-b]pyridin-3-yl)-ethyl]-1,2,3,4-tetrahydro-isoquinolin-7-yl}-N-hydroxy-acrylamide, (E)-N-Hydroxy-3-(2-phenethyl-1,2,3,4-tetrahydro-isoquinolin-7-yl)-acrylamide, (E)-3-[2-(2-Cyclohexyl-ethyl)-1,2,3,4-tetrahydro-isoquinolin-7-yl]-N-hydroxy-acrylamide, (E)-N-Hydroxy-3-{2-[2-(1-methyl-3-phenyl-1H-pyrazol-4-yl)-ethyl]-1,2,3,4-tetrahydro-isoquinolin-7-yl}-acrylamide, (E)-N-Hydroxy-3-{2-[2-(3-phenyl-1H-pyrazol-4-yl)-ethyl]-1,2,3,4-tetrahydro-isoquinolin-7-yl}-acrylamide, (E)-N-Hydroxy-3-{2-[2-(2-phenyl-2H-pyrazol-3-yl)-ethyl]-1,2,3,4-tetrahydro-isoquinolin-7-yl}-acrylamide, (E)-N-Hydroxy-3-{2-[2-(5-methyl-1-phenyl-1H-pyrazol-4-yl)-ethyl]-1,2,3,4-tetrahydro-isoquinolin-7-yl}-acrylamide, (E)-N-Hydroxy-3-{2-[2-(1,3,5-trimethyl-1H-pyrazol-4-yl)-ethyl]-1,2,3,4-tetrahydro-isoquinolin-7-yl}-acrylamide, (E)-3-(2-Benzyl-1,2,3,4-tetrahydro-isoquinolin-7-yl)-N-hydroxy-acrylamide, (E)-3-[2-(4-Dimethylamino-benzoyl)-1,2,3,4-tetrahydro-isoquinolin-7-yl]-N-hydroxy-acrylamide, (E)-3-(2-Benzenesulfonyl-1,2,3,4-tetrahydro-isoquinolin-7-yl)-N-hydroxy-acrylamide, (E)-N-Hydroxy-3-[2-(3-methoxy-benzenesulfonyl)-1,2,3,4-tetrahydro-isoquinolin-7-yl]-acrylamide, (E)-N-Hydroxy-3-[2-(4-methoxy-benzenesulfonyl)-1,2,3,4-tetrahydro-isoquinolin-7-yl]-acrylamide, (E)-N-Hydroxy-3-[2-(4-methoxy-benzoyl)-1,2,3,4-tetrahydro-isoquinolin-7-yl]-acrylamide, (E)-N-Hydroxy-3-[2-(3-methoxy-benzoyl)-1,2,3,4-tetrahydro-isoquinolin-7-yl]-acrylamide, N-Hydroxy-3-[2-(3-methoxy-benzoyl)-1,2,3,4-tetrahydro-isoquinolin-7-yl]-propionamide, (E)-N-Hydroxy-3-{2-[2-(2-methyl-1H-indol-3-yl)-acetyl]-1,2,3,4-tetrahydro-isoquinolin-7-yl}-acrylamide, (E)-N-Hydroxy-3-{2-[2-(1H-indol-3-yl)-ethyl]-1,2,3,4-tetrahydro-isoquinolin-6-yl}-acrylamide, (E)-N-Hydroxy-3-[2-(2-pyrazolo[1,5-a]pyridin-3-yl-ethyl)-1,2,3,4-tetrahydro-isoquinolin-6-yl]-acrylamide, (E)-N-Hydroxy-3-{2-[2-(2-methyl-1H-indol-3-yl)-ethyl]-1,2,3,4-tetrahydro-isoquinolin-6-yl}-acrylamide, (E)-N-Hydroxy-3-{2-[2-(2-methyl-1H-pyrrolo[2,3-b]pyridin-3-yl)-ethyl]-1,2,3,4-tetrahydro-isoquinolin-6-yl}acrylamide, (E)-N-Hydroxy-3-{2-[2-(2-methyl-pyrazolo[1,5-a]pyridin-3-yl)-ethyl]-1,2,3,4-tetrahydro-isoquinolin-6-yl}-acrylamide, (E)-3-{2-[2-(2-Ethyl-pyrazolo[1,5-a]pyridin-3-yl)-ethyl]-1,2,3,4-tetrahydro-isoquinolin-6-yl}-N-hydroxy-acrylamide, 3-{2-[2-(2-Ethyl-pyrazolo[1,5-a]pyridin-3-yl)-ethyl]-1,2,3,4-tetrahydro-isoquinolin-6-yl}-N-hydroxy-propionamide, (E)-3-{2-[2-(2-tert-Butyl-1H-indol-3-yl)-ethyl]-1,2,3,4-tetrahydro-isoquinolin-6-yl}-N-hydroxy-acrylamide, (E)-3-{2-[2-(2-tert-Butyl-1H-pyrrolo[2,3-b]pyridin-3-yl)-ethyl]-1,2,3,4-tetrahydro-isoquinolin-6-yl}-N-hydroxy-acrylamide, (E)-N-Hydroxy-3-(2-phenethyl-1,2,3,4-tetrahydro-isoquinolin-6-yl)-acrylamide, (E)-3-[2-(2-Cyclohexyl-ethyl)-1,2,3,4-tetrahydro-isoquinolin-6-yl]-N-hydroxy-acrylamide, (E)-3-(2-Benzyl-1,2,3,4-tetrahydro-isoquinolin-6-yl)-N-hydroxy-acrylamide, (E)-N-Hydroxy-3-{2-[2-(3-methyl-5-phenyl-isoxazol-4-yl)-ethyl]-1,2,3,4-tetrahydro-isoquinolin-6-yl}-acrylamide, (E)-N-Hydroxy-3-{2-[2-(3-methyl-1-phenyl-1H-pyrazol-4-yl)-ethyl]-1,2,3,4-tetrahydro-isoquinolin-6-yl}-acrylamide, (E)-N-Hydroxy-3-{2-[2-(1-methyl-3-phenyl-1H-pyrazol-4-yl)-ethyl]-1,2,3,4-tetrahydro-isoquinolin-6-yl}-acrylamide, (E)-N-Hydroxy-3-{2-[2-(3-phenyl-1H-pyrazol-4-yl)-ethyl]-1,2,3,4-tetrahydro-isoquinolin-6-yl}-acrylamide, (E)-N-Hydroxy-3-{2-[2-(1,3,5-trimethyl-1H-pyrazol-4-yl)-ethyl]-1,2,3,4-tetrahydro-isoquinolin-6-yl}-acrylamide, (E)-N-Hydroxy-3-{2-[2-(2-phenyl-2H-pyrazol-3-yl)-ethyl]-1,2,3,4-tetrahydro-isoquinolin-6-yl}-acrylamide, (E)-3-(2-Benzenesulfonyl-1,2,3,4-tetrahydro-isoquinolin-6-yl)-N-hydroxy-acrylamide, (E)-N-Hydroxy-3-[2-(4-methoxy-benzenesulfonyl)-1,2,3,4-tetrahydro-isoquinolin-6-yl]-acrylamide, (E)-N-Hydroxy-3-[2-(3-methoxy-benzenesulfonyl)-1,2,3,4-tetrahydro-isoquinolin-6-yl]-acrylamide, N-Hydroxy-3-[2-(3-methoxy-benzenesulfonyl)-1,2,3,4-tetrahydro-isoquinolin-6-yl]-propionamide, (E)-N-Hydroxy-3-{2-[2-(5-methyl-1-phenyl-1H-pyrazol-4-yl)-ethyl]-1,2,3,4-tetrahydro-isoquinolin-6-yl}-acrylamide, (E)-3-[2-(4-Dimethylamino-benzoyl)-1,2,3,4-tetrahydro-isoquinolin-6-yl]-N-hydroxy-acrylamide, 3-[2-(4-Dimethylamino-benzoyl)-1,2,3,4-tetrahydro-isoquinolin-6-yl]-N-hydroxy-propionamide, (E)-N-Hydroxy-3-[2-(3-methoxy-benzoyl)-1,2,3,4-tetrahydro-isoquinolin-6-yl]-acrylamide, (E)-N-Hydroxy-3-[2-(4-methoxy-benzoyl)-1,2,3,4-tetrahydro-isoquinolin-6-yl]-acrylamide, (E)-N-Hydroxy-3-{2-[2-(2-methyl-1H-indol-3-yl)-acetyl]-1,2,3,4-tetrahydro-isoquinolin-6-yl}-acrylamide, (E)-N-Hydroxy-3-{3-[2-(2-methyl-1H-indol-3-yl)-ethyl]-2,3,4,5-tetrahydro-1H-benzo[d]azepin-7-yl}-acrylamide, (E)-3-{3-[2-(2-tert-Butyl-1H-indol-3-yl)-ethyl]-2,3,4,5-tetrahydro-1H-benzo[d]azepin-7-yl}-N-hydroxy-acrylamide, (E)-N-Hydroxy-3-[3-(2-pyrazolo[1,5-a]pyridin-3-yl-ethyl)-2,3,4,5-tetrahydro-1H-benzo[d]azepin-7-yl]-acrylamide, (E)-N-Hydroxy-3-{3-[2-(2-methyl-pyrazolo[1,5-a]pyridin-3-yl)-ethyl]-2,3,4,5-tetrahydro-1H-benzo[d]azepin-7-yl}-acrylamide, (E)-3-{3-[2-(2-Ethyl-pyrazolo[1,5-a]pyridin-3-yl)-ethyl]2,3,4,5-tetrahydro-1H-benzo[d]azepin-7-yl}-N-hydroxy-acrylamide, (E)-N-Hydroxy-3-{3-[2-(2-methyl-1H-pyrrolo[2,3-b]pyridin-3-yl)-ethyl]-2,3,4,5-tetrahydro-1H-benzo[d]azepin-7-yl}-acrylamide, (E)-3-{3-[2-(2-tert-Butyl-1H-pyrrolo[2,3-b]pyridin-3-yl)-ethyl]-2,3,4,5-tetrahydro-1H-benzo[d]azepin-7-yl}-N-hydroxy-acrylamide, (E)-N-Hydroxy-3-{3-[2-(1,3,5-trimethyl-1H-pyrazol-4-yl)-ethyl]-2,3,4,5-tetrahydro-1H-benzo[d]azepin-7-yl}-acrylamide, (E)-N-Hydroxy-3-{3-[2-(1-methyl-3-phenyl-1H-pyrazol-4-yl)-ethyl]-2,3,4,5-tetrahydro-1H-benzo[d]azepin-7-yl}-acrylamide, (E)-N-Hydroxy-3-{3-[2-(3-phenyl-1H-pyrazol-4-yl)-ethyl]-2,3,4,5-tetrahydro-1H-benzo[d]azepin-7-yl}-acrylamide, (E)-N-Hydroxy-3-{3-[2-(2-phenyl-2H-pyrazol-3-yl)-ethyl]-2,3,4,5-tetrahydro-1H-benzo[d]azepin-7-yl}-acrylamide, (E) —N-Hydroxy-3-{3-[2-(3-methyl-1-phenyl-1H-pyrazol-4-yl)-ethyl]-2,3,4,5-tetrahydro-1H-benzo[d]azepin-7-yl}-acrylamide, (E)-N-Hydroxy-3-{3-[2-(3-methyl-5-phenyl-isoxazol-4-yl)-ethyl]-2,3,4,5-tetrahydro-1H-benzo[d]azepin-7-yl}-acrylamide, (E)-N-Hydroxy-3-{2-[2-(2-methyl-1H-indol-3-yl)-ethyl]-2,3,4,5-tetrahydro-1H-benzo[c]azepin-7-yl}-acrylamide, (E)-3-{2-[2-(2-tert-Butyl-1H-indol-3-yl)-ethyl]-2,3,4,5-tetrahydro-1H-benzo[c]azepin-7-yl}-N-hydroxy-acrylamide, (E)-N-Hydroxy-3-[2-(2-pyrazolo[1,5-a]pyridin-3-yl-ethyl)-2,3,4,5-tetrahydro-1H-benzo[c]azepin-7-yl]-acrylamide, (E)-N-Hydroxy-3-{2-[2-(2-methyl-pyrazolo[1,5-a]pyridin-3-yl)-ethyl]-2,3,4,5-tetrahydro-1H-benzo[c]azepin-7-yl}-acrylamide, (E)-3-{2-[2-(2-Ethyl-pyrazolo[1,5-a]pyridin-3-yl)-ethyl]-2,3,4,5-tetrahydro-1H-benzo[c]azepin-7-yl}-N-hydroxy-acrylamide, (E)-N-Hydroxy-3-{2-[2-(2-methyl-1H-pyrrolo[2,3-b]pyridin-3-yl)-ethyl]-2,3,4,5-tetrahydro-1H-benzo[c]azepin-7-yl}-acrylamide, (E)-3-{2-[2-(2-tert-Butyl-1H-pyrrolo[2,3-b]pyridin-3-yl)-ethyl]-2,3,4,5-tetrahydro-1H-benzo[c]azepin-7-yl}-N-hydroxy-acrylamide, (E)-N-Hydroxy-3-{2-[2-(1,3,5-trimethyl-1H-pyrazol-4-yl)-ethyl]-2,3,4,5-tetrahydro-1H-benzo[c]azepin-7-yl}-acrylamide, (E)-N-Hydroxy-3-{2-[2-(1-methyl-3-phenyl-1H-pyrazol-4-yl)-ethyl]-2,3,4,5-tetrahydro-1H-benzo[c]azepin-7-yl}-acrylamide, (E)-N-Hydroxy-3-{2-[2-(3-phenyl-1H-pyrazol-4-yl)-ethyl]-2,3,4,5-tetrahydro-1H-benzo[c]azepin-7-yl}-acrylamide, (E)-N-Hydroxy-3-{2-[2-(2-phenyl-2H-pyrazol-3-yl)-ethyl]-2,3,4,5-tetrahydro-1H-benzo[c]azepin-7-yl}-acrylamide, (E) —N-Hydroxy-3-{2-[2-(3-methyl-1-phenyl-1H-pyrazol-4-yl)-ethyl]-2,3,4,5-tetrahydro-1H-benzo[c]azepin-7-yl}-acrylamide, (E)-N-Hydroxy-3-{2-[2-(3-methyl-5-phenyl-isoxazol-4-yl)-ethyl]-2,3,4,5-tetrahydro-1H-benzo[c]azepin-7-yl}-acrylamide

5. A method of treating a deacetylase-associated disorder comprising administering to a subject in need thereof a pharmaceutically acceptable amount of a compound such that the deacetylase-associated disorder is treated, wherein the compound is of the Formula I.

6. The method of claim 5, wherein the compound is selected from the group consisting of (E)-3-(2-Benzyl-2,3-dihydro-1H-isoindol-5-yl)-N-hydroxy-acrylamide, 5-((E)-2-Hydroxycarbamoyl-vinyl)-1,3-dihydro-isoindole-2-carboxylic acid tert-butyl ester, (E)-3-[2-(2,2-Dimethyl-propionyl)-2,3-dihydro-1H-isoindol-5-yl]-N-hydroxy-acrylamide, (E)-N-Hydroxy-3-(2-methanesulfonyl-2,3-dihydro-1H-isoindol-5-yl)-acryl amide, (E)-3-(2-Benzenesulfonyl-2,3-dihydro-1H-isoindol-5-yl)-N-hydroxy-acrylamide, (E)-N-Hydroxy-3-[2-(pyridine-4-sulfonyl)-2,3-dihydro-1H-isoindol-5-yl]-acrylamide, (E)-N-Hydroxy-3-[2-(pyridine-3-sulfonyl)-2,3-dihydro-1H-isoindol-5-yl]-acrylamide, (E)-N-Hydroxy-3-[2-(toluene-4-sulfonyl)-2,3-dihydro-1H-isoindol-5-yl]-acrylamide, (E)-N-Hydroxy-3-[2-(4-methoxy-benzenesulfonyl)-2,3-dihydro-1H-isoindol-5-yl]-acrylamide, (E)-N-Hydroxy-3-[2-(4-trifluoromethyl-benzenesulfonyl)-2,3-dihydro-1H-isoindol-5-yl]-acrylamide, (E)-N-Hydroxy-3-[2-(toluene-3-sulfonyl)-2,3-dihydro-1H-isoindol-5-yl]-acrylamide, (E)-N-Hydroxy-3-[2-(3-trifluoromethyl-benzenesulfonyl)-2,3-dihydro-1H-isoindol-5-yl]-acrylamide, (E)-N-Hydroxy-3-[2-(3-methoxy-benzenesulfonyl)-2,3-dihydro-1H-isoindol-5-yl]-acrylamide, (E)-N-Hydroxy-3-[2-(toluene-2-sulfonyl)-2,3-dihydro-1H-isoindol-5-yl]-acrylamide, (E)-N-Hydroxy-3-[2-(1-methyl-1H-imidazole-4-sulfonyl)-2,3-dihydro-1H-isoindol-5-yl]-acrylamide, (E)-N-Hydroxy-3-[2-(3-trifluoromethyl-benzoyl)-2,3-dihydro-1H-isoindol-5-yl]-acrylamide, (E)-N-Hydroxy-3-[2-(pyridine-3-carbonyl)-2,3-dihydro-1H-isoindol-5-yl]-acrylamide, (E)-N-Hydroxy-3-[2-(thiophene-2-carbonyl)-2,3-dihydro-1H-isoindol-5-yl]-acrylamide, (E)-3-[2-(Butane-1-sulfonyl)-2,3-dihydro-1H-isoindol-5-yl]-N-hydroxy-acrylamide, (E)-N-Hydroxy-3-(2-phenylmethanesulfonyl-2,3-dihydro-1H-isoindol-5-yl)-acrylamide, (E)-3-(2-Cyclohexanecarbonyl-2,3-dihydro-1H-isoindol-5-yl)-N-hydroxy-acrylamide, (E)-3-(2-Cyclopentanecarbonyl-2,3-dihydro-1H-isoindol-5-yl)-N-hydroxy-acrylamide, (E)-3-(2-Cyclopropanecarbonyl-2,3-dihydro-1H-isoindol-5-yl)-N-hydroxy-acrylamide, (E)-3-(2-Cyclopropanesulfonyl-2,3-dihydro-1H-isoindol-5-yl)-N-hydroxy-acrylamide, (E)-N-Hydroxy-3-{2-[2-(2-methyl-1H-indol-3-yl)-acetyl]-2,3-dihydro-1H-isoindol-5-yl}-acrylamide, (E)-N-Hydroxy-3-{2-[2-(1H-indol-3-yl)-ethyl]-2,3-dihydro-1H-isoindol-5-yl}-acrylamide, (E)-N-Hydroxy-3-[2-(2-pyrazolo[1,5-a]pyridin-3-yl-ethyl)-2,3-dihydro-1H-isoindol-5-yl]-acrylamide, (E)-N-Hydroxy-3-{2-[2-(2-methyl-1H-indol-3-yl)-ethyl]-2,3-dihydro-1H-isoindol-5-yl}-acrylamide, (E)-N-Hydroxy-3-{2-[2-(2-methyl-pyrazolo[1,5-a]pyridin-3-yl)-ethyl]-2,3-dihydro-1H-isoindol-5-yl}-acrylamide, (E)-N-Hydroxy-3-{2-[2-(2-methyl-1H-pyrrolo[2,3-b]pyridin-3-yl)-ethyl]-2,3-dihydro-1H-isoindol-5-yl}-acrylamide, (E)-3-{2-[2-(2-Ethyl-pyrazolo[1,5-a]pyridin-3-yl)-ethyl]-2,3-dihydro-1H-isoindol-5-yl}-N-hydroxy-acrylamide, (E)-3-{2-[2-(2-tert-Butyl-1H-indol-3-yl)-ethyl]-2,3-dihydro-1H-isoindol-5-yl}-N-hydroxy-acrylamide, (E)-N-Hydroxy-3-{2-[2-(2-methyl-1H-pyrrolo[2,3-b]pyridin-3-yl)-ethyl]-2,3-dihydro-1H-isoindol-5-yl}-acrylamide, (E)-3-{2-[2-(2-tert-Butyl-1H-pyrrolo[2,3-b]pyridin-3-yl)-ethyl]-2,3-dihydro-1H-isoindol-5-yl}-N-hydroxy-acrylamide, (E)-N-Hydroxy-3-(2-phenethyl-2,3-dihydro-1H-isoindol-5-yl)-acrylamide, (E) —N-Hydroxy-3-{2-[2-(3-methyl-5-phenyl-isoxazol-4-yl)-ethyl]-2,3-dihydro-1H-isoindol-5-yl}-acrylamide, (E)-3-[2-(2-Cyclohexyl-ethyl)-2,3-dihydro-1H-isoindol-5-yl]-N-hydroxy-acrylamide, (E)-N-Hydroxy-3-{2-[2-(1H-indol-3-yl)-ethyl]-1,2,3,4-tetrahydro-isoquinolin-7-yl}-acrylamide, N-Hydroxy-3-{2-[2-(1H-indol-3-yl)-ethyl]-1,2,3,4-tetrahydro-isoquinolin-7-yl}-propionamide, (E)-N-Hydroxy-3-[2-(2-pyrazolo[1,5-a]pyridin-3-yl-ethyl)-1,2,3,4-tetrahydro-isoquinolin-7-yl]-acrylamide, (E)-N-Hydroxy-3-{2-[2-(3-methyl-5-phenyl-isoxazol-4-yl)-ethyl]-1,2,3,4-tetrahydro-isoquinolin-7-yl}-acrylamide, (E)-N-Hydroxy-3-{2-[2-(2-methyl-1H-indol-3-yl)-ethyl]-1,2,3,4-tetrahydro-isoquinolin-7-yl}-acrylamide, (E)-N-Hydroxy-3-{2-[2-(2-methyl-1H-pyrrolo[2,3-b]pyridin-3-yl)-ethyl]-1,2,3,4-tetrahydro-isoquinolin-7-yl}-acrylamide, (E)-N-Hydroxy-3-{2-[2-(2-methyl-pyrazolo[1,5-a]pyridin-3-yl)-ethyl]-1,2,3,4-tetrahydro-isoquinolin-7-yl}-acrylamide, (E)-3-{2-[2-(2-Ethyl-pyrazolo[1,5-a]pyridin-3-yl)-ethyl]-1,2,3,4-tetrahydro-isoquinolin-7-yl}-N-hydroxy-acrylamide, (E)-3-{2-[2-(2-tert-Butyl-1H-indol-3-yl)-ethyl]-1,2,3,4-tetrahydro-isoquinolin-7-yl}-N-hydroxy-acrylamide, (E)-3-{2-[2-(2-tert-Butyl-1H-pyrrolo[2,3-b]pyridin-3-yl)-ethyl]-1,2,3,4-tetrahydro-isoquinolin-7-yl}-N-hydroxy-acrylamide, (E)-N-Hydroxy-3-(2-phenethyl-1,2,3,4-tetrahydro-isoquinolin-7-yl)-acrylamide, (E)-3-[2-(2-Cyclohexyl-ethyl)-1,2,3,4-tetrahydro-isoquinolin-7-yl]-N-hydroxy-acrylamide, (E)-N-Hydroxy-3-{2-[2-(1-methyl-3-phenyl-1H-pyrazol-4-yl)-ethyl]-1,2,3,4-tetrahydro-isoquinolin-7-yl}-acrylamide, (E)-N-Hydroxy-3-{2-[2-(3-phenyl-1H-pyrazol-4-yl)-ethyl]-1,2,3,4-tetrahydro-isoquinolin-7-yl}-acrylamide, (E)-N-Hydroxy-3-{2-[2-(2-phenyl-2H-pyrazol-3-yl)-ethyl]-1,2,3,4-tetrahydro-isoquinolin-7-yl}-acrylamide, (E)-N-Hydroxy-3-{2-[2-(5-methyl-1-phenyl-1H-pyrazol-4-yl)-ethyl]-1,2,3,4-tetrahydro-isoquinolin-7-yl}-acrylamide, (E)-N-Hydroxy-3-{2-[2-(1,3,5-trimethyl-1H-pyrazol-4-yl)-ethyl]-1,2,3,4-tetrahydro-isoquinolin-7-yl}-acrylamide, (E)-3-(2-Benzyl-1,2,3,4-tetrahydro-isoquinolin-7-yl)-N-hydroxy-acrylamide, (E)-3-[2-(4-Dimethylamino-benzoyl)-1,2,3,4-tetrahydro-isoquinolin-7-yl]-N-hydroxy-acrylamide, (E)-3-(2-Benzenesulfonyl-1,2,3,4-tetrahydro-isoquinolin-7-yl)-N-hydroxy-acrylamide, (E)-N-Hydroxy-3-[2-(3-methoxy-benzenesulfonyl)-1,2,3,4-tetrahydro-isoquinolin-7-yl]-acrylamide, (E)-N-Hydroxy-3-[2-(4-methoxy-benzenesulfonyl)-1,2,3,4-tetrahydro-isoquinolin-7-yl]-acrylamide, (E)-N-Hydroxy-3-[2-(4-methoxy-benzoyl)-1,2,3,4-tetrahydro-isoquinolin-7-yl]-acrylamide, (E)-N-Hydroxy-3-[2-(3-methoxy-benzoyl)-1,2,3,4-tetrahydro-isoquinolin-7-yl]-acrylamide, N-Hydroxy-3-[2-(3-methoxy-benzoyl)-1,2,3,4-tetrahydro-isoquinolin-7-yl]-propionamide, (E)-N-Hydroxy-3-{2-[2-(2-methyl-1H-indol-3-yl)-acetyl]-1,2,3,4-tetrahydro-isoquinolin-7-yl}-acrylamide, (E)-N-Hydroxy-3-{2-[2-(1H-indol-3-yl)-ethyl]-1,2,3,4-tetrahydro-isoquinolin-6-yl}-acrylamide, (E)-N-Hydroxy-3-[2-(2-pyrazolo[1,5-a]pyridin-3-yl-ethyl)-1,2,3,4-tetrahydro-isoquinolin-6-yl]-acrylamide, (E)-N-Hydroxy-3-{2-[2-(2-methyl-1H-indol-3-yl)-ethyl]-1,2,3,4-tetrahydro-isoquinolin-6-yl}-acrylamide, (E)-N-Hydroxy-3-{2-[2-(2-methyl-1H-pyrrolo[2,3-b]pyridin-3-yl)-ethyl]-1,2,3,4-tetrahydro-isoquinolin-6-yl}-acrylamide, (E)-N-Hydroxy-3-{2-[2-(2-methyl-pyrazolo[1,5-a]pyridin-3-yl)-ethyl]-1,2,3,4-tetrahydro-isoquinolin-6-yl}-acrylamide, (E)-3-{2-[2-(2-Ethyl-pyrazolo[1,5-a]pyridin-3-yl)-ethyl]-1,2,3,4-tetrahydro-isoquinolin-6-yl}-N-hydroxy-acrylamide, 3-{2-[2-(2-Ethyl-pyrazolo[1,5-a]pyridin-3-yl)-ethyl]-1,2,3,4-tetrahydro-isoquinolin-6-yl}-N-hydroxy-propionamide, (E)-3-{2-[2-(2-tert-Butyl-1H-indol-3-yl)-ethyl]-1,2,3,4-tetrahydro-isoquinolin-6-yl}-N-hydroxy-acrylamide, (E)-3-{2-[2-(2-tert-Butyl-1H-pyrrolo[2,3-b]pyridin-3-yl)-ethyl]-1,2,3,4-tetrahydro-isoquinolin-6-yl}-N-hydroxy-acrylamide, (E)-N-Hydroxy-3-(2-phenethyl-1,2,3,4-tetrahydro-isoquinolin-6-yl)-acrylamide, (E)-3-[2-(2-Cyclohexyl-ethyl)-1,2,3,4-tetrahydro-isoquinolin-6-yl]-N-hydroxy-acrylamide, (E)-3-(2-Benzyl-1,2,3,4-tetrahydro-isoquinolin-6-yl)-N-hydroxy-acrylamide, (E)-N-Hydroxy-3-{2-[2-(3-methyl-5-phenyl-isoxazol-4-yl)-ethyl]-1,2,3,4-tetrahydro-isoquinolin-6-yl}-acrylamide, (E)-N-Hydroxy-3-{2-[2-(3-methyl-1-phenyl-1H-pyrazol-4-yl)-ethyl]-1,2,3,4-tetrahydro-isoquinolin-6-yl}-acrylamide, (E)-N-Hydroxy-3-{2-[2-(1-methyl-3-phenyl-1H-pyrazol-4-yl)-ethyl]-1,2,3,4-tetrahydro-isoquinolin-6-yl}-acrylamide, (E)-N-Hydroxy-3-{2-[2-(3-phenyl-1H-pyrazol-4-yl)-ethyl]-1,2,3,4-tetrahydro-isoquinolin-6-yl}-acrylamide, (E)-N-Hydroxy-3-{2-[2-(1,3,5-trimethyl-1H-pyrazol-4-yl)-ethyl]-1,2,3,4-tetrahydro-isoquinolin-6-yl}-acrylamide, (E)-N-Hydroxy-3-{2-[2-(2-phenyl-2H-pyrazol-3-yl)-ethyl]-1,2,3,4-tetrahydro-isoquinolin-6-yl}-acrylamide, (E)-3-(2-Benzenesulfonyl-1,2,3,4-tetrahydro-isoquinolin-6-yl)-N-hydroxy-acrylamide, (E)-N-Hydroxy-3-[2-(4-methoxy-benzenesulfonyl)-1,2,3,4-tetrahydro-isoquinolin-6-yl]-acrylamide, (E)-N-Hydroxy-3-[2-(3-methoxy-benzenesulfonyl)-1,2,3,4-tetrahydro-isoquinolin-6-yl]-acrylamide, N-Hydroxy-3-[2-(3-methoxy-benzenesulfonyl)-1,2,3,4-tetrahydro-isoquinolin-6-yl]-propionamide, (E)-N-Hydroxy-3-{2-[2-(5-methyl-1-phenyl-1H-pyrazol-4-yl)-ethyl]-1,2,3,4-tetrahydro-isoquinolin-6-yl}-acrylamide, (E)-3-[2-(4-Dimethylamino-benzoyl)-1,2,3,4-tetrahydro-isoquinolin-6-yl]-N-hydroxy-acrylamide, 3-[2-(4-Dimethylamino-benzoyl)-1,2,3,4-tetrahydro-isoquinolin-6-yl]-N-hydroxy-propionamide, (E)-N-Hydroxy-3-[2-(3-methoxy-benzoyl)-1,2,3,4-tetrahydro-isoquinolin-6-yl]-acrylamide, (E)-N-Hydroxy-3-[2-(4-methoxy-benzoyl)-1,2,3,4-tetrahydro-isoquinolin-6-yl]-acrylamide, (E)-N-Hydroxy-3-{2-[2-(2-methyl-1H-indol-3-yl)-acetyl]-1,2,3,4-tetrahydro-isoquinolin-6-yl}-acrylamide, (E)-N-Hydroxy-3-{3-[2-(2-methyl-1H-indol-3-yl)-ethyl]-2,3,4,5-tetrahydro-1H-benzo[d]azepin-7-yl}-acrylamide, (E)-3-{3-[2-(2-tert-Butyl-1H-indol-3-yl)-ethyl]-2,3,4,5-tetrahydro-1H-benzo[d]azepin-7-yl}-N-hydroxy-acrylamide, (E)-N-Hydroxy-3-[3-(2-pyrazolo[1,5-a]pyridin-3-yl-ethyl)-2,3,4,5-tetrahydro-1H-benzo[d]azepin-7-yl]-acrylamide, (E)-N-Hydroxy-3-{3-[2-(2-methyl-pyrazolo[1,5-a]pyridin-3-yl)-ethyl]-2,3,4,5-tetrahydro-1H-benzo[d]azepin-7-yl}-acrylamide, (E)-3-{3-[2-(2-Ethyl-pyrazolo[1,5-a]pyridin-3-yl)-ethyl]-2,3,4,5-tetrahydro-1H-benzo[d]azepin-7-yl}-N-hydroxy-acrylamide, (E)-N-Hydroxy-3-{3-[2-(2-methyl-1H-pyrrolo[2,3-b]pyridin-3-yl)-ethyl]-2,3,4,5-tetrahydro-1H-benzo[d]azepin-7-yl}-acrylamide, (E)-3-{3-[2-(2-tert-Butyl-1H-pyrrolo[2,3-b]pyridin-3-yl)-ethyl]-2,3,4,5-tetrahydro-1H-benzo[d]azepin-7-yl}-N-hydroxy-acrylamide, (E)-N-Hydroxy-3-{3-[2-(1,3,5-trimethyl-1H-pyrazol-4-yl)-ethyl]-2,3,4,5-tetrahydro-1H-benzo[d]azepin-7-yl}-acrylamide, (E)-N-Hydroxy-3-{3-[2-(1-methyl-3-phenyl-1H-pyrazol-4-yl)-ethyl]-2,3,4,5-tetrahydro-1H-benzo[d]azepin-7-yl}-acrylamide, (E)-N-Hydroxy-3-{3-[2-(3-phenyl-1H-pyrazol-4-yl)-ethyl]-2,3,4,5-tetrahydro-1H-benzo[d]azepin-7-yl}-acrylamide, (E)-N-Hydroxy-3-{3-[2-(2-phenyl-2H-pyrazol-3-yl)-ethyl]-2,3,4,5-tetrahydro-1H-benzo[d]azepin-7-yl}-acrylamide, (E) —N-Hydroxy-3-{3-[2-(3-methyl-1-phenyl-1H-pyrazol-4-yl)-ethyl]-2,3,4,5-tetrahydro-1H-benzo[d]azepin-7-yl}-acrylamide, (E)-N-Hydroxy-3-{3-[2-(3-methyl-5-phenyl-isoxazol-4-yl)-ethyl]-2,3,4,5tetrahydro-1H-benzo[d]azepin-7-yl}-acrylamide, (E)-N-Hydroxy-3-{2-[2-(2-methyl-1H-indol-3-yl)-ethyl]-2,3,4,5-tetrahydro-1H-benzo[c]azepin-7-yl}-acrylamide, (E)-3-{2-[2-(2-tert-Butyl-1H-indol-3-yl)-ethyl]-2,3,4,5-tetrahydro-1H-benzo[c]azepin-7-yl}-N-hydroxy-acrylamide, (E)-N-Hydroxy-3-[2-(2-pyrazolo[1,5-a]pyridin-3-yl-ethyl)-2,3,4,5-tetrahydro-1H-benzo[c]azepin-7-yl]-acrylamide, (E)-N-Hydroxy-3-{2-[2-(2-methyl-pyrazolo[1,5-a]pyridin-3-yl)-ethyl]-2,3,4,5-tetrahydro-1H-benzo[c]azepin-7-yl}-acrylamide, (E)-3-{2-[2-(2-Ethyl-pyrazolo[1,5-a]pyridin-3-yl)-ethyl]-2,3,4,5-tetrahydro-1H-benzo[c]azepin-7-yl}-N-hydroxy-acrylamide, (E)-N-Hydroxy-3-{2-[2-(2-methyl-1H-pyrrolo[2,3-b]pyridin-3-yl)-ethyl]-2,3,4,5-tetrahydro-1H-benzo[c]azepin-7-yl}-acrylamide, (E)-3-{2-[2-(2-tert-Butyl-1H-pyrrolo[2,3-b]pyridin-3-yl)-ethyl]-2,3,4,5-tetrahydro-1H-benzo[c]azepin-7-yl}-N-hydroxy-acrylamide, (E)-N-Hydroxy-3-{2-[2-(1,3,5-trimethyl-1H-pyrazol-4-yl)-ethyl]-2,3,4,5-tetrahydro-1H-benzo[c]azepin-7-yl}-acrylamide, (E)-N-Hydroxy-3-{2-[2-(1-methyl-3-phenyl-1H-pyrazol-4-yl)-ethyl]-2,3,4,5-tetrahydro-1H-benzo[c]azepin-7-yl}-acrylamide, (E)-N-Hydroxy-3-{2-[2-(3-phenyl-1H-pyrazol-4-yl)-ethyl]-2,3,4,5-tetrahydro-1H-benzo[c]azepin-7-yl}-acrylamide, (E)-N-Hydroxy-3-{2-[2-(2-phenyl-2H-pyrazol-3-yl)-ethyl]-2,3,4,5-tetrahydro-1H-benzo[c]azepin-7-yl}-acrylamide, (E)-N-Hydroxy-3-{2-[2-(3-methyl-1-phenyl-1H-pyrazol-4-yl)-ethyl]-2,3,4,5-tetrahydro-1H-benzo[c]azepin-7-yl}-acrylamide, (E)-N-Hydroxy-3-{2-[2-(3-methyl-5-phenyl-isoxazol-4-yl)-ethyl]-2,3,4,5-tetrahydro-1H-benzo[c]azepin-7-yl}-acrylamide.

7. The method of claim 5, wherein the deacetylase-associated disorder is selected from a group consisting of a proliferative disease, a hyperproliferative disease, a disease of the immune system, a disease of the central nervous system, a disease associated with misexpression of a gene, or peripheral nervous system.

8. The method of claim 7, wherein the deacetylase-associated disorder is an HDAC dependent disease, wherein the HDAC is selected from the group of HDAC1, HDAC2, HDAC3, HDAC4, HDAC5, HDAC6, HDAC7, HDAC8, HDAC9, HDAC10 and HDAC11, or any combinations thereof.

9. The method of claim 8, wherein the proliferative disease is selected from the group consisting of a hyperproliferative disease, a benign tumor, a malignant tumor, a carcinoma of the brain, kidney, liver, adrenal gland, bladder, breast, stomach, ovaries, esophagus, colon, rectum, prostate, pancreas, lung, vagina, or thyroid, a sarcoma, glioblastomas, multiple myeloma, gastrointestinal cancer, colon carcinoma, colorectal adenoma, a tumor of the neck or head, an epidermal hyperproliferation, psoriasis, prostate hyperplasia, a neoplasia, mammary carcinoma, and a leukemia.

10. The method of claims 5, wherein the deacetylase-associated disorder is triggered by persistent angiogenesis, such as psoriasis; Kaposi's sarcoma; restenosis, e.g., stent-induced restenosis; endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

11. The method of claim 5, wherein the deacetylase-associated disorder is a disease of the immune system.

12. The method of claim 5, wherein the hyperproliferative disease is selected from the group consisting of leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

13. A method of treating a proliferative disease comprising administering to a subject in need thereof a pharmaceutically acceptable amount of a compound such that the proliferative disease is treated, wherein the compound is of the Formula I.

14. The method of claim 13, wherein the compound is selected from the group consisting of (E)-3-(2-Benzyl-2,3-dihydro-1H-isoindol-5-yl)-N-hydroxy-acrylamide, 5-((E)-2-Hydroxycarbamoyl-vinyl)-1,3-dihydro-isoindole-2-carboxylic acid tert-butyl ester, (E)-3-[2-(2,2-Dimethyl-propionyl)-2,3-dihydro-1H-isoindol-5-yl]-N-hydroxy-acrylamide, (E)-N-Hydroxy-3-(2-methanesulfonyl-2,3-dihydro-1H-isoindol-5-yl)-acryl amide, (E)-3-(2-Benzenesulfonyl-2,3-dihydro-1H-isoindol-5-yl)-N-hydroxy-acrylamide, (E)-N-Hydroxy-3-[2-(pyridine-4-sulfonyl)-2,3-dihydro-1H-isoindol-5-yl]-acrylamide, (E)-N-Hydroxy-3-[2-(pyridine-3-sulfonyl)-2,3-dihydro-1H-isoindol-5-yl]-acrylamide, (E)-N-Hydroxy-3-[2-(toluene-4-sulfonyl)-2,3-dihydro-1H-isoindol-5-yl]-acrylamide, (E)-N-Hydroxy-3-[2-(4-methoxy-benzenesulfonyl)-2,3-dihydro-1H-isoindol-5-yl]-acrylamide, (E)-N-Hydroxy-3-[2-(4-trifluoromethyl-benzenesulfonyl)-2,3-dihydro-1H-isoindol-5-yl]-acrylamide, (E)-N-Hydroxy-3-[2-(toluene-3-sulfonyl)-2,3-dihydro-1H-isoindol-5-yl]-acrylamide, (E)-N-Hydroxy-3-[2-(3-trifluoromethyl-benzenesulfonyl)-2,3-dihydro-1H-isoindol-5-yl]-acrylamide, (E)-N-Hydroxy-3-[2-(3-methoxy-benzenesulfonyl)-2,3-dihydro-1H-isoindol-5-yl]-acrylamide, (E)-N-Hydroxy-3-[2-(toluene-2-sulfonyl)-2,3-dihydro-1H-isoindol-5-yl]-acrylamide, (E)-N-Hydroxy-3-[2-(1-methyl-1H-imidazole-4-sulfonyl)-2,3-dihydro-1H-isoindol-5-yl]-acrylamide, (E)-N-Hydroxy-3-[2-(3-trifluoromethyl-benzoyl)-2,3-dihydro-1H-isoindol-5-yl]-acrylamide, (E)-N-Hydroxy-3-[2-(pyridine-3-carbonyl)-2,3-dihydro-1H-isoindol-5-yl]-acrylamide, (E)-N-Hydroxy-3-[2-(thiophene-2-carbonyl)-2,3-dihydro-1H-isoindol-5-yl]-acrylamide, (E)-3-[2-(Butane-1-sulfonyl)-2,3-dihydro-1H-isoindol-5-yl]-N-hydroxy-acrylamide, (E)-N-Hydroxy-3-(2-phenylmethanesulfonyl-2,3-dihydro-1H-isoindol-5-yl)-acrylamide, (E)-3-(2-Cyclohexanecarbonyl-2,3-dihydro-1H-isoindol-5-yl)-N-hydroxy-acrylamide, (E)-3-(2-Cyclopentanecarbonyl-2,3-dihydro-1H-isoindol-5-yl)-N-hydroxy-acrylamide, (E)-3-(2-Cyclopropanecarbonyl-2,3-dihydro-1H-isoindol-5-yl)-N-hydroxy-acrylamide, (E)-3-(2-Cyclopropanesulfonyl-2,3-dihydro-1H-isoindol-5-yl)-N-hydroxy-acrylamide, (E)-N-Hydroxy-3-{2-[2-(2-methyl-1H-indol-3-yl)-acetyl]-2,3-dihydro-1H-isoindol-5-yl}-acrylamide, (E)-N-Hydroxy-3-{2-[2-(1H-indol-3-yl)-ethyl]-2,3-dihydro-1H-isoindol-5-yl}-acrylamide, (E)-N-Hydroxy-3-[2-(2-pyrazolo[1,5-a]pyridin-3-yl-ethyl)-2,3-dihydro-1H-isoindol-5-yl]-acrylamide, (E)-N-Hydroxy-3-{2-[2-(2-methyl-1H-indol-3-yl)-ethyl]-2,3-dihydro-1H-isoindol-5-yl}-acrylamide, (E)-N-Hydroxy-3-{2-[2-(2-methyl-pyrazolo[1,5-a]pyridin-3-yl)-ethyl]-2,3-dihydro-1H-isoindol-5-yl}-acrylamide, (E)-N-Hydroxy-3-{2-[2-(2-methyl-1H-pyrrolo[2,3-b]pyridin-3-yl)-ethyl]-2,3-dihydro-1H-isoindol-5-yl}-acrylamide, (E)-3-{2-[2-(2-Ethyl-pyrazolo[1,5-a]pyridin-3-yl)-ethyl]-2,3-dihydro-1H-isoindol-5-yl}-N-hydroxy-acrylamide, (E)-3-{2-[2-(2-tert-Butyl-1H-indol-3-yl)-ethyl]-2,3-dihydro-1H-isoindol-5-yl}-N-hydroxy-acrylamide, (E)-N-Hydroxy-3-{2-[2-(2-methyl-1H-pyrrolo[2,3-b]pyridin-3-yl)-ethyl]-2,3-dihydro-1H-isoindol-5-yl}-acrylamide, (E)-3-{2-[2-(2-tert-Butyl-1H-pyrrolo[2,3-b]pyridin-3-yl)-ethyl]-2,3-dihydro-1H-isoindol-5-yl}-N-hydroxy-acrylamide, (E)-N-Hydroxy-3-(2-phenethyl-2,3-dihydro-1H-isoindol-5-yl)-acrylamide, (E) —N-Hydroxy-3-{2-[2-(3-methyl-5-phenyl-isoxazol-4-yl)-ethyl]-2,3-dihydro-1H-isoindol-5-yl}-acrylamide, (E)-3-[2-(2-Cyclohexyl-ethyl)-2,3-dihydro-1H-isoindol-5-yl]-N-hydroxy-acrylamide, (E)-N-Hydroxy-3-{2-[2-(1H-indol-3-yl)-ethyl]-1,2,3,4-tetrahydro-isoquinolin-7-yl}-acrylamide, N-Hydroxy-3-{2-[2-(1H-indol-3-yl)-ethyl]-1,2,3,4-tetrahydro-isoquinolin-7-yl}-propionamide, (E)-N-Hydroxy-3-[2-(2-pyrazolo[1,5-a]pyridin-3-yl-ethyl)-1,2,3,4-tetrahydro-isoquinolin-7-yl]-acrylamide, (E)-N-Hydroxy-3-{2-[2-(3-methyl-5-phenyl-isoxazol-4-yl)-ethyl]-1,2,3,4-tetrahydro-isoquinolin-7-yl}-acrylamide, (E)-N-Hydroxy-3-{2-[2-(2-methyl-1H-indol-3-yl)-ethyl]-1,2,3,4-tetrahydro-isoquinolin-7-yl}-acrylamide, (E)-N-Hydroxy-3-{2-[2-(2-methyl-1H-pyrrolo[2,3-b]pyridin-3-yl)-ethyl]-1,2,3,4-tetrahydro-isoquinolin-7-yl}-acrylamide, (E)-N-Hydroxy-3-{2-[2-(2-methyl-pyrazolo[1,5-a]pyridin-3-yl)-ethyl]-1,2,3,4-tetrahydro-isoquinolin-7-yl}-acrylamide, (E)-3-{2-[2-(2-Ethyl-pyrazolo[1,5-a]pyridin-3-yl)-ethyl]-1,2,3,4-tetrahydro-isoquinolin-7-yl}-N-hydroxy-acrylamide, (E)-3-{2-[2-(2-tert-Butyl-1H-indol-3-yl)-ethyl]-1,2,3,4-tetrahydro-isoquinolin-7-yl}-N-hydroxy-acrylamide, (E)-3-{2-[2-(2-tert-Butyl-1H-pyrrolo[2,3-b]pyridin-3-yl)-ethyl]1,2,3,4-tetrahydro-isoquinolin-7-yl}-N-hydroxy-acrylamide, (E)-N-Hydroxy-3-(2-phenethyl-1,2,3,4-tetrahydro-isoquinolin-7-yl)-acrylamide, (E)-3-[2-(2-Cyclohexyl-ethyl)-1,2,3,4-tetrahydro-isoquinolin-7-yl]-N-hydroxy-acrylamide, (E)-N-Hydroxy-3-{2-[2-(1-methyl-3-phenyl-1H-pyrazol-4-yl)-ethyl]-1,2,3,4-tetrahydro-isoquinolin-7-yl}-acrylamide, (E)-N-Hydroxy-3-{2-[2-(3-phenyl-1H-pyrazol-4-yl)-ethyl]-1,2,3,4-tetrahydro-isoquinolin-7-yl}-acrylamide, (E)-N-Hydroxy-3-{2-[2-(2-phenyl-2H-pyrazol-3-yl)-ethyl]-1,2,3,4-tetrahydro-isoquinolin-7-yl}-acrylamide, (E)-N-Hydroxy-3-{2-[2-(5-methyl-1-phenyl-1H-pyrazol-4-yl)-ethyl]-1,2,3,4-tetrahydro-isoquinolin-7-yl}-acrylamide, (E)-N-Hydroxy-3-{2-[2-(1,3,5-trimethyl-1H-pyrazol-4-yl)-ethyl]-1,2,3,4-tetrahydro-isoquinolin-7-yl}-acrylamide, (E)-3-(2-Benzyl-1,2,3,4-tetrahydro-isoquinolin-7-yl)-N-hydroxy-acrylamide, (E)-3-[2-(4-Dimethylamino-benzoyl)-1,2,3,4-tetrahydro-isoquinolin-7-yl]-N-hydroxy-acrylamide, (E)-3-(2-Benzenesulfonyl-1,2,3,4-tetrahydro-isoquinolin-7-yl)-N-hydroxy-acrylamide, (E)-N-Hydroxy-3-[2-(3-methoxy-benzenesulfonyl)-1,2,3,4-tetrahydro-isoquinolin-7-yl]-acrylamide, (E)-N-Hydroxy-3-[2-(4-methoxy-benzenesulfonyl)-1,2,3,4-tetrahydro-isoquinolin-7-yl]-acrylamide, (E)-N-Hydroxy-3-[2-(4-methoxy-benzoyl)-1,2,3,4-tetrahydro-isoquinolin-7-yl]-acrylamide, (E)-N-Hydroxy-3-[2-(3-methoxy-benzoyl)-1,2,3,4-tetrahydro-isoquinolin-7-yl]-acrylamide, N-Hydroxy-3-[2-(3-methoxy-benzoyl)-1,2,3,4-tetrahydro-isoquinolin-7-yl]-propionamide, (E)-N-Hydroxy-3-{2-[2-(2-methyl-1H-indol-3-yl)-acetyl]-1,2,3,4-tetrahydro-isoquinolin-7-yl}-acrylamide, (E)-N-Hydroxy-3-{2-[2-(1H-indol-3-yl)-ethyl]-1,2,3,4-tetrahydro-isoquinolin-6-yl}-acrylamide, (E)-N-Hydroxy-3-[2-(2-pyrazolo[1,5-a]pyridin-3-yl-ethyl)-1,2,3,4-tetrahydro-isoquinolin-6-yl]-acrylamide, (E)-N-Hydroxy-3-{2-[2-(2-methyl-1H-indol-3-yl)-ethyl]-1,2,3,4-tetrahydro-isoquinolin-6-yl}-acrylamide, (E)-N-Hydroxy-3-{2-[2-(2-methyl-1H-pyrrolo[2,3-b]pyridin-3-yl)-ethyl]-1,2,3,4-tetrahydro-isoquinolin-6-yl}-acrylamide, (E)-N-Hydroxy-3-{2-[2-(2-methyl-pyrazolo[1,5-a]pyridin-3-yl)-ethyl]-1,2,3,4-tetrahydro-isoquinolin-6-yl}-acrylamide, (E)-3-{2-[2-(2-Ethyl-pyrazolo[1,5-a]pyridin-3-yl)-ethyl]-1,2,3,4-tetrahydro-isoquinolin-6-yl}-N-hydroxy-acrylamide, 3-{2-[2-(2-Ethyl-pyrazolo[1,5-a]pyridin-3-yl)-ethyl]-1,2,3,4-tetrahydro-isoquinolin-6-yl}-N-hydroxy-propionamide, (E)-3-{2-[2-(2-tert-Butyl-1H-indol-3-yl)-ethyl]-1,2,3,4-tetrahydro-isoquinolin-6-yl}-N-hydroxy-acrylamide, (E)-3-{2-[2-(2-tert-Butyl-1H-pyrrolo[2,3-b]pyridin-3-yl)-ethyl]-1,2,3,4-tetrahydro-isoquinolin-6-yl}-N-hydroxy-acrylamide, (E)-N-Hydroxy-3-(2-phenethyl-1,2,3,4-tetrahydro-isoquinolin-6-yl)-acrylamide, (E)-3-[2-(2-Cyclohexyl-ethyl)-1,2,3,4-tetrahydro-isoquinolin-6-yl]-N-hydroxy-acrylamide, (E)-3-(2-Benzyl-1,2,3,4-tetrahydro-isoquinolin-6-yl)-N-hydroxy-acrylamide, (E)-N-Hydroxy-3-{2-[2-(3-methyl-5-phenyl-isoxazol-4-yl)-ethyl]-1,2,3,4-tetrahydro-isoquinolin-6-yl}-acrylamide, (E)-N-Hydroxy-3-{2-[2-(3-methyl-1-phenyl-1H-pyrazol-4-yl)-ethyl]-1,2,3,4-tetrahydro-isoquinolin-6-yl}-acrylamide, (E)-N-Hydroxy-3-{2-[2-(1-methyl-3-phenyl-1H-pyrazol-4-yl)-ethyl]-1,2,3,4-tetrahydro-isoquinolin-6-yl}-acrylamide, (E)-N-Hydroxy-3-{2-[2-(3-phenyl-1H-pyrazol-4-yl)-ethyl]-1,2,3,4-tetrahydro-isoquinolin-6-yl}-acrylamide, (E)-N-Hydroxy-3-{2-[2-(1,3,5-trimethyl-1H-pyrazol-4-yl)-ethyl]-1,2,3,4-tetrahydro-isoquinolin-6-yl}-acrylamide, (E)-N-Hydroxy-3-{2-[2-(2-phenyl-2H-pyrazol-3-yl)-ethyl]-1,2,3,4-tetrahydro-isoquinolin-6-yl}-acrylamide, (E)-3-(2-Benzenesulfonyl-1,2,3,4-tetrahydro-isoquinolin-6-yl)-N-hydroxy-acrylamide, (E)-N-Hydroxy-3-[2-(4-methoxy-benzenesulfonyl)-1,2,3,4-tetrahydro-isoquinolin-6-yl]-acrylamide, (E)-N-Hydroxy-3-[2-(3-methoxy-benzenesulfonyl)-1,2,3,4-tetrahydro-isoquinolin-6-yl]-acrylamide, N-Hydroxy-3-[2-(3-methoxy-benzenesulfonyl)-1,2,3,4-tetrahydro-isoquinolin-6-yl]-propionamide, (E)-N-Hydroxy-3-{2-[2-(5-methyl-1-phenyl-1H-pyrazol-4-yl)-ethyl]-1,2,3,4-tetrahydro-isoquinolin-6-yl}-acrylamide, (E)-3-[2-(4-Dimethylamino-benzoyl)-1,2,3,4-tetrahydro-isoquinolin-6-yl]-N-hydroxy-acrylamide, 3-[2-(4-Dimethylamino-benzoyl)-1,2,3,4-tetrahydro-isoquinolin-6-yl]-N-hydroxy-propionamide, (E)-N-Hydroxy-3-[2-(3-methoxy-benzoyl)-1,2,3,4-tetrahydro-isoquinolin-6-yl]-acrylamide, (E)-N-Hydroxy-3-[2-(4-methoxy-benzoyl)-1,2,3,4-tetrahydro-isoquinolin-6-yl]-acrylamide, (E)-N-Hydroxy-3-{2-[2-(2-methyl-1H-indol-3-yl)-acetyl]-1,2,3,4-tetrahydro-isoquinolin-6-yl}-acrylamide, (E)-N-Hydroxy-3-{3-[2-(2-methyl-1H-indol-3-yl)-ethyl]-2,3,4,5-tetrahydro-1H-benzo[d]azepin-7-yl}-acrylamide, (E)-3-{3-[2-(2-tert-Butyl-1H-indol-3-yl)-ethyl]-2,3,4,5-tetrahydro-1H-benzo[d]azepin-7-yl}-N-hydroxy-acrylamide, (E)-N-Hydroxy-3-[3-(2-pyrazolo[1,5-a]pyridin-3-yl-ethyl)-2,3,4,5-tetrahydro-1H-benzo[d]azepin-7-yl]-acrylamide, (E)-N-Hydroxy-3-{3-[2-(2-methyl-pyrazolo[1,5-a]pyridin-3-yl)-ethyl]-2,3,4,5-tetrahydro-1H-benzo[d]azepin-7-yl}-acrylamide, (E)-3-{3-[2-(2-Ethyl-pyrazolo[1,5-a]pyridin-3-yl)-ethyl]-2,3,4,5-tetrahydro-1H-benzo[d]azepin-7-yl}-N-hydroxy-acrylamide, (E)-N-Hydroxy-3-{3-[2-(2-methyl-1H-pyrrolo[2,3-b]pyridin-3-yl)-ethyl]-2,3,4,5-tetrahydro-1H-benzo[d]azepin-7-yl}-acrylamide, (E)-3-{3-[2-(2-tert-Butyl-1H-pyrrolo[2,3-b]pyridin-3-yl)-ethyl]-2,3,4,5-tetrahydro-1H-benzo[d]azepin-7-yl}-N-hydroxy-acrylamide, (E)-N-Hydroxy-3-{3-[2-(1,3,5-trimethyl-1H-pyrazol-4-yl)-ethyl]-2,3,4,5-tetrahydro-1H-benzo[d]azepin-7-yl}-acrylamide, (E)-N-Hydroxy-3-{3-[2-(1-methyl-3-phenyl-1H-pyrazol-4-yl)-ethyl]-2,3,4,5-tetrahydro-1H-benzo[d]azepin-7-yl}-acrylamide, (E)-N-Hydroxy-3-{3-[2-(3-phenyl-1H-pyrazol-4-yl)-ethyl]-2,3,4,5-tetrahydro-1H-benzo[d]azepin-7-yl}-acrylamide, (E)-N-Hydroxy-3-{3-[2-(2-phenyl-2H-pyrazol-3-yl)-ethyl]-2,3,4,5-tetrahydro-1H-benzo[d]azepin-7-yl}-acrylamide, (E) —N-Hydroxy-3-{3-[2-(3-methyl-1-phenyl-1H-pyrazol-4-yl)-ethyl]-2,3,4,5-tetrahydro-1H-benzo[d]azepin-7-yl}-acrylamide, (E)-N-Hydroxy-3-{3-[2-(3-methyl-5-phenyl-isoxazol-4-yl)-ethyl]-2,3,4,5tetrahydro-1H-benzo[d]azepin-7-yl}-acrylamide, (E)-N-Hydroxy-3-{2-[2-(2-methyl-1H-indol-3-yl)-ethyl]-2,3,4,5-tetrahydro-1H-benzo[c]azepin-7-yl}-acrylamide, (E)-3-{2-[2-(2-tert-Butyl-1H-indol-3-yl)-ethyl]-2,3,4,5-tetrahydro-1H-benzo[c]azepin-7-yl}-N-hydroxy-acrylamide, (E)-N-Hydroxy-3-[2-(2-pyrazolo[1,5-a]pyridin-3-yl-ethyl)-2,3,4,5-tetrahydro-1H-benzo[c]azepin-7-yl]-acrylamide, (E)-N-Hydroxy-3-{2-[2-(2-methyl-pyrazolo[1,5-a]pyridin-3-yl)-ethyl]-2,3,4,5-tetrahydro-1H-benzo[c]azepin-7-yl}-acrylamide, (E)-3-{2-[2-(2-Ethyl-pyrazolo[1,5-a]pyridin-3-yl)-ethyl]-2,3,4,5-tetrahydro-1H-benzo[c]azepin-7-yl}-N-hydroxy-acrylamide, (E)-N-Hydroxy-3-{2-[2-(2-methyl-1H-pyrrolo[2,3-b]pyridin-3-yl)-ethyl]-2,3,4,5-tetrahydro-1H-benzo[c]azepin-7-yl}-acrylamide, (E)-3-{2-[2-(2-tert-Butyl-1H-pyrrolo[2,3-b]pyridin-3-yl)-ethyl]-2,3,4,5-tetrahydro-1H-benzo[c]azepin-7-yl}-N-hydroxy-acrylamide, (E)-N-Hydroxy-3-{2-[2-(1,3,5-trimethyl-1H-pyrazol-4-yl)-ethyl]-2,3,4,5-tetrahydro-1H-benzo[c]azepin-7-yl}-acrylamide, (E)-N-Hydroxy-3-{2-[2-(1-methyl-3-phenyl-1H-pyrazol-4-yl)-ethyl]-2,3,4,5-tetrahydro-1H-benzo[c]azepin-7-yl}-acrylamide, (E)-N-Hydroxy-3-{2-[2-(3-phenyl-1H-pyrazol-4-yl)-ethyl]-2,3,4,5-tetrahydro-1H-benzo[c]azepin-7-yl}-acrylamide, (E)-N-Hydroxy-3-{2-[2-(2-phenyl-2H-pyrazol-3-yl)-ethyl]-2,3,4,5-tetrahydro-1H-benzo[c]azepin-7-yl}-acrylamide, (E) —N-Hydroxy-3-{2-[2-(3-methyl-1-phenyl-1H-pyrazol-4-yl)-ethyl]-2,3,4,5-tetrahydro-1H-benzo[c]azepin-7-yl}-acrylamide, (E)-N-Hydroxy-3-{2-[2-(3-methyl-5-phenyl-isoxazol-4-yl)-ethyl]-2,3,4,5-tetrahydro-1H-benzo[c]azepin-7-yl}-acrylamide.

15. The method of claim 14, wherein the proliferative disease is selected from the group consisting of a hyperproliferative disease, a benign tumor, a malignant tumor, a carcinoma of the brain, kidney, liver, adrenal gland, bladder, breast, stomach, ovaries, esophagus, colon, rectum, prostate, pancreas, lung, vagina, or thyroid, a sarcoma, glioblastomas, multiple myeloma, gastrointestinal cancer, colon carcinoma, colorectal adenoma, a tumor of the neck or head, an epidermal hyperproliferation, psoriasis, prostate hyperplasia, a neoplasia, mammary carcinoma, and a leukemia.

16. The method of claim 15, wherein the hyperproliferative disease is selected from the group consisting of leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

17. A packaged deacetylase-associated disorder treatment, comprising a deacetylase-modulating compound of the Formula I.

18. The packaged deacetylase-associated disorder treatment of claim 17, wherein the compound is selected from the group consisting of (E)-3-(2-Benzyl-2,3-dihydro-1H-isoindol-5-yl)-N-hydroxy-acrylamide, 5-((E)-2-Hydroxycarbamoyl-vinyl)-1,3-dihydro-isoindole-2-carboxylic acid tert-butyl ester, (E)-3-[2-(2,2-Dimethyl-propionyl)-2,3-dihydro-1H-isoindol-5-yl]-N-hydroxy-acrylamide, (E)-N-Hydroxy-3-(2-methanesulfonyl-2,3-dihydro-1H-isoindol-5-yl)-acryl amide, (E)-3-(2-Benzenesulfonyl-2,3-dihydro-1H-isoindol-5-yl)-N-hydroxy-acrylamide, (E)-N-Hydroxy-3-[2-(pyridine-4-sulfonyl)-2,3-dihydro-1H-isoindol-5-yl]-acrylamide, (E)-N-Hydroxy-3-[2-(pyridine-3-sulfonyl)-2,3-dihydro-1H-isoindol-5-yl]-acrylamide, (E)-N-Hydroxy-3-[2-(toluene-4-sulfonyl)-2,3-dihydro-1H-isoindol-5-yl]-acrylamide, (E)-N-Hydroxy-3-[2-(4-methoxy-benzenesulfonyl)-2,3-dihydro-1H-isoindol-5-yl]-acrylamide, (E)-N-Hydroxy-3-[2-(4-trifluoromethyl-benzenesulfonyl)-2,3-dihydro-1H-isoindol-5-yl]-acrylamide, (E)-N-Hydroxy-3-[2-(toluene-3-sulfonyl)-2,3-dihydro-1H-isoindol-5-yl]-acrylamide, (E)-N-Hydroxy-3-[2-(3-trifluoromethyl-benzenesulfonyl)-2,3-dihydro-1H-isoindol-5-yl]-acrylamide, (E)-N-Hydroxy-3-[2-(3-methoxy-benzenesulfonyl)-2,3-dihydro-1H-isoindol-5-yl]-acrylamide, (E)-N-Hydroxy-3-[2-(toluene-2-sulfonyl)-2,3-dihydro-1H-isoindol-5-yl]-acrylamide, (E)-N-Hydroxy-3-[2-(1-methyl-1H-imidazole-4-sulfonyl)-2,3-dihydro-1H-isoindol-5-yl]-acrylamide, (E)-N-Hydroxy-3-[2-(3-trifluoromethyl-benzoyl)-2,3-dihydro-1H-isoindol-5-yl]-acrylamide, (E)-N-Hydroxy-3-[2-(pyridine-3-carbonyl)-2,3-dihydro-1H-isoindol-5-yl]-acrylamide, (E)-N-Hydroxy-3-[2-(thiophene-2-carbonyl)-2,3-dihydro-1H-isoindol-5-yl]-acrylamide, (E)-3-[2-(Butane-1-sulfonyl)-2,3-dihydro-1H-isoindol-5-yl]-N-hydroxy-acrylamide, (E)-N-Hydroxy-3-(2-phenylmethanesulfonyl-2,3-dihydro-1H-isoindol-5-yl)-acrylamide, (E)-3-(2-Cyclohexanecarbonyl-2,3-dihydro-1H-isoindol-5-yl)-N-hydroxy-acrylamide, (E)-3-(2-Cyclopentanecarbonyl-2,3-dihydro-1H-isoindol-5-yl)-N-hydroxy-acrylamide, (E)-3-(2-Cyclopropanecarbonyl-2,3-dihydro-1H-isoindol-5-yl)-N-hydroxy-acrylamide, (E)-3-(2-Cyclopropanesulfonyl-2,3-dihydro-1H-isoindol-5-yl)-N-hydroxy-acrylamide, (E)-N-Hydroxy-3-{2-[2-(2-methyl-1H-indol-3-yl)-acetyl]-2,3-dihydro-1H-isoindol-5-yl}-acrylamide, (E)-N-Hydroxy-3-{2-[2-(1H-indol-3-yl)-ethyl]-2,3-dihydro-1H-isoindol-5-yl}-acrylamide, (E)-N-Hydroxy-3-[2-(2-pyrazolo[1,5-a]pyridin-3-yl-ethyl)-2,3-dihydro-1H-isoindol-5-yl]-acrylamide, (E)-N-Hydroxy-3-{2-[2-(2-methyl-1H-indol-3-yl)-ethyl]-2,3-dihydro-1H-isoindol-5-yl}-acrylamide, (E)-N-Hydroxy-3-{2-[2-(2-methyl-pyrazolo[1,5-a]pyridin-3-yl)-ethyl]-2,3-dihydro-1H-isoindol-5-yl}-acrylamide, (E)-N-Hydroxy-3-{2-[2-(2-methyl-1H-pyrrolo[2,3-b]pyridin-3-yl)-ethyl]-2,3-dihydro-1H-isoindol-5-yl}-acrylamide, (E)-3-{2-[2-(2-Ethyl-pyrazolo[1,5-a]pyridin-3-yl)-ethyl]-2,3-dihydro-1H-isoindol-5-yl}-N-hydroxy-acrylamide, (E)-3-{2-[2-(2-tert-Butyl-1H-indol-3-yl)-ethyl]-2,3-dihydro-1H-isoindol-5-yl}-N-hydroxy-acrylamide, (E)-N-Hydroxy-3-{2-[2-(2-methyl-1H-pyrrolo[2,3-b]pyridin-3-yl)-ethyl]-2,3-dihydro-1H-isoindol-5-yl}-acrylamide, (E)-3-{2-[2-(2-tert-Butyl-1H-pyrrolo[2,3-b]pyridin-3-yl)-ethyl]-2,3-dihydro-1H-isoindol-5-yl}-N-hydroxy-acrylamide, (E)-N-Hydroxy-3-(2-phenethyl-2,3-dihydro-1H-isoindol-5-yl)-acrylamide, (E)-N-Hydroxy-3-{2-[2-(3-methyl-5-phenyl-isoxazol-4-yl)-ethyl]-2,3-dihydro-1H-isoindol-5-yl}-acrylamide, (E)-3-[2-(2-Cyclohexyl-ethyl)-2,3-dihydro-1H-isoindol-5-yl]-N-hydroxy-acrylamide, (E)-N-Hydroxy-3-{2-[2-(1H-indol-3-yl)-ethyl]-1,2,3,4-tetrahydro-isoquinolin-7-yl}-acrylamide, N-Hydroxy-3-{2-[2-(1H-indol-3-yl)-ethyl]-1,2,3,4-tetrahydro-isoquinolin-7-yl}-propionamide, (E)-N-Hydroxy-3-[2-(2-pyrazolo[1,5-a]pyridin-3-yl-ethyl)-1,2,3,4-tetrahydro-isoquinolin-7-yl]-acrylamide, (E)-N-Hydroxy-3-{2-[2-(3-methyl-5-phenyl-isoxazol-4-yl)-ethyl]-1,2,3,4-tetrahydro-isoquinolin-7-yl}-acrylamide, (E)-N-Hydroxy-3-{2-[2-(2-methyl-1H-indol-3-yl)-ethyl]-1,2,3,4-tetrahydro-isoquinolin-7-yl}-acrylamide, (E)-N-Hydroxy-3-{2-[2-(2-methyl-1H-pyrrolo[2,3-b]pyridin-3-yl)-ethyl]-1,2,3,4-tetrahydro-isoquinolin-7-yl}-acrylamide, (E) —N-Hydroxy-3-{2-[2-(2-methyl-pyrazolo[1,5-a]pyridin-3-yl)-ethyl]-1,2,3,4-tetrahydro-isoquinolin-7-yl}-acrylamide, (E)-3-{2-[2-(2-Ethyl-pyrazolo[1,5-a]pyridin-3-yl)-ethyl]-1,2,3,4-tetrahydro-isoquinolin-7-yl}-N-hydroxy-acrylamide, (E)-3-{2-[2-(2-tert-Butyl-1H-indol-3-yl)-ethyl]-1,2,3,4-tetrahydro-isoquinolin-7-yl}-N-hydroxy-acrylamide, (E)-3-{2-[2-(2-tert-Butyl-1H-pyrrolo[2,3-b]pyridin-3-yl)-ethyl]-1,2,3,4-tetrahydro-isoquinolin-7-yl}-N-hydroxy-acrylamide, (E)-N-Hydroxy-3-(2-phenethyl-1,2,3,4-tetrahydro-isoquinolin-7-yl)-acrylamide, (E)-3-[2-(2-Cyclohexyl-ethyl)-1,2,3,4-tetrahydro-isoquinolin-7-yl]-N-hydroxy-acrylamide, (E)-N-Hydroxy-3-{2-[2-(1-methyl-3-phenyl-1H-pyrazol-4-yl)-ethyl]-1,2,3,4-tetrahydro-isoquinolin-7-yl}-acrylamide, (E)-N-Hydroxy-3-{2-[2-(3-phenyl-1H-pyrazol-4-yl)-ethyl]-1,2,3,4-tetrahydro-isoquinolin-7-yl}-acrylamide, (E)-N-Hydroxy-3-{2-[2-(2-phenyl-2H-pyrazol-3-yl)-ethyl]-1,2,3,4-tetrahydro-isoquinolin-7-yl}-acrylamide, (E)-N-Hydroxy-3-{2-[2-(5-methyl-1-phenyl-1H-pyrazol-4-yl)-ethyl]-1,2,3,4-tetrahydro-isoquinolin-7-yl}-acrylamide, (E)-N-Hydroxy-3-{2-[2-(1,3,5-trimethyl-1H-pyrazol-4-yl)-ethyl]-1,2,3,4-tetrahydro-isoquinolin-7-yl}-acrylamide, (E)-3-(2-Benzyl-1,2,3,4-tetrahydro-isoquinolin-7-yl)-N-hydroxy-acrylamide, (E)-3-[2-(4-Dimethylamino-benzoyl)-1,2,3,4-tetrahydro-isoquinolin-7-yl]-N-hydroxy-acrylamide, (E)-3-(2-Benzenesulfonyl-1,2,3,4-tetrahydro-isoquinolin-7-yl)-N-hydroxy-acrylamide, (E)-N-Hydroxy-3-[2-(3-methoxy-benzenesulfonyl)-1,2,3,4-tetrahydro-isoquinolin-7-yl]-acrylamide, (E)-N-Hydroxy-3-[2-(4-methoxy-benzenesulfonyl)-1,2,3,4-tetrahydro-isoquinolin-7-yl]-acrylamide, (E)-N-Hydroxy-3-[2-(4-methoxy-benzoyl)-1,2,3,4-tetrahydro-isoquinolin-7-yl]-acrylamide, (E)-N-Hydroxy-3-[2-(3-methoxy-benzoyl)-1,2,3,4-tetrahydro-isoquinolin-7-yl]-acrylamide, N-Hydroxy-3-[2-(3-methoxy-benzoyl)-1,2,3,4-tetrahydro-isoquinolin-7-yl]-propionamide, (E)-N-Hydroxy-3-{2-[2-(2-methyl-1H-indol-3-yl)-acetyl]-1,2,3,4-tetrahydro-isoquinolin-7-yl}-acrylamide, (E)-N-Hydroxy-3-{2-[2-(1H-indol-3-yl)-ethyl]-1,2,3,4-tetrahydro-isoquinolin-6-yl}-acrylamide, (E)-N-Hydroxy-3-[2-(2-pyrazolo[1,5-a]pyridin-3-yl-ethyl)-1,2,3,4-tetrahydro-isoquinolin-6-yl]-acrylamide, (E)-N-Hydroxy-3-{2-[2-(2-methyl-1H-indol-3-yl)-ethyl]-1,2,3,4-tetrahydro-isoquinolin-6-yl}-acrylamide, (E)-N-Hydroxy-3-{2-[2-(2-methyl-1H-pyrrolo[2,3-b]pyridin-3-yl)-ethyl]-1,2,3,4-tetrahydro-isoquinolin-6-yl}-acrylamide, (E)-N-Hydroxy-3-{2-[2-(2-methyl-pyrazolo[1,5-a]pyridin-3-yl)-ethyl]-1,2,3,4-tetrahydro-isoquinolin-6-yl}-acrylamide, (E)-3-{2-[2-(2-Ethyl-pyrazolo[1,5-a]pyridin-3-yl)-ethyl]-1,2,3,4-tetrahydro-isoquinolin-6-yl}-N-hydroxy-acrylamide, 3-{2-[2-(2-Ethyl-pyrazolo[1,5-a]pyridin-3-yl)-ethyl]-1,2,3,4-tetrahydro-isoquinolin-6-yl}-N-hydroxy-propionamide, (E)-3-{2-[2-(2-tert-Butyl-1H-indol-3-yl)-ethyl]-1,2,3,4-tetrahydro-isoquinolin-6-yl}-N-hydroxy-acrylamide, (E)-3-{2-[2-(2-tert-Butyl-1H-pyrrolo[2,3-b]pyridin-3-yl)-ethyl]-1,2,3,4-tetrahydro-isoquinolin-6-yl}-N-hydroxy-acrylamide, (E)-N-Hydroxy-3-(2-phenethyl-1,2,3,4-tetrahydro-isoquinolin-6-yl)-acrylamide, (E)-3-[2-(2-Cyclohexyl-ethyl)-1,2,3,4-tetrahydro-isoquinolin-6-yl]-N-hydroxy-acrylamide, (E)-3-(2-Benzyl-1,2,3,4-tetrahydro-isoquinolin-6-yl)-N-hydroxy-acrylamide, (E)-N-Hydroxy-3-{2-[2-(3-methyl-5-phenyl-isoxazol-4-yl)-ethyl]-1,2,3,4-tetrahydro-isoquinolin-6-yl}-acrylamide, (E)-N-Hydroxy-3-{2-[2-(3-methyl-1-phenyl-1H-pyrazol-4-yl)-ethyl]-1,2,3,4-tetrahydro-isoquinolin-6-yl}-acrylamide, (E)-N-Hydroxy-3-{2-[2-(1-methyl-3-phenyl-1H-pyrazol-4-yl)-ethyl]-1,2,3,4-tetrahydro-isoquinolin-6-yl}-acrylamide, (E)-N-Hydroxy-3-{2-[2-(3-phenyl-1H-pyrazol-4-yl)-ethyl]-1,2,3,4-tetrahydro-isoquinolin-6-yl}-acrylamide, (E)-N-Hydroxy-3-{2-[2-(1,3,5-trimethyl-1H-pyrazol-4-yl)-ethyl]-1,2,3,4-tetrahydro-isoquinolin-6-yl}-acrylamide, (E)-N-Hydroxy-3-{2-[2-(2-phenyl-2H-pyrazol-3-yl)-ethyl]-1,2,3,4-tetrahydro-isoquinolin-6-yl}-acrylamide, (E)-3-(2-Benzenesulfonyl-1,2,3,4-tetrahydro-isoquinolin-6-yl)-N-hydroxy-acrylamide, (E)-N-Hydroxy-3-[2-(4-methoxy-benzenesulfonyl)-1,2,3,4-tetrahydro-isoquinolin-6-yl]-acrylamide, (E)-N-Hydroxy-3-[2-(3-methoxy-benzenesulfonyl)-1,2,3,4-tetrahydro-isoquinolin-6-yl]-acrylamide, N-Hydroxy-3-[2-(3-methoxy-benzenesulfonyl)-1,2,3,4-tetrahydro-isoquinolin-6-yl]-propionamide, (E)-N-Hydroxy-3-{2-[2-(5-methyl-1-phenyl-1H-pyrazol-4-yl)-ethyl]-1,2,3,4-tetrahydro-isoquinolin-6-yl}-acrylamide, (E)-3-[2-(4-Dimethylamino-benzoyl)-1,2,3,4-tetrahydro-isoquinolin-6-yl]N-hydroxy-acrylamide, 3-[2-(4-Dimethylamino-benzoyl)-1,2,3,4-tetrahydro-isoquinolin-6-yl]-N-hydroxy-propionamide, (E)-N-Hydroxy-3-[2-(3-methoxy-benzoyl)-1,2,3,4-tetrahydro-isoquinolin-6-yl]-acrylamide, (E)-N-Hydroxy-3-[2-(4-methoxy-benzoyl)-1,2,3,4-tetrahydro-isoquinolin-6-yl]-acrylamide, (E)-N-Hydroxy-3-{2-[2-(2-methyl-1H-indol-3-yl)-acetyl]-1,2,3,4-tetrahydro-isoquinolin-6-yl}-acrylamide, (E)-N-Hydroxy-3-{3-[2-(2-methyl-1H-indol-3-yl)-ethyl]-2,3,4,5-tetrahydro-1H-benzo[d]azepin-7-yl}-acrylamide, (E)-3-{3-[2-(2-tert-Butyl-1H-indol-3-yl)-ethyl]-2,3,4,5-tetrahydro-1H-benzo[d]azepin-7-yl}-N-hydroxy-acrylamide, (E)-N-Hydroxy-3-[3-(2-pyrazolo[1,5-a]pyridin-3-yl-ethyl)-2,3,4,5-tetrahydro-1H-benzo[d]azepin-7-yl]-acrylamide, (E)-N-Hydroxy-3-{3-[2-(2-methyl-pyrazolo[1,5-a]pyridin-3-yl)-ethyl]-2,3,4,5-tetrahydro-1H-benzo[d]azepin-7-yl}-acrylamide, (E)-3-{3-[2-(2-Ethyl-pyrazolo[1,5-a]pyridin-3-yl)-ethyl]-2,3,4,5-tetrahydro-1H-benzo[d]azepin-7-yl}-N-hydroxy-acrylamide, (E)-N-Hydroxy-3-{3-[2-(2-methyl-1H-pyrrolo[2,3-b]pyridin-3-yl)-ethyl]-2,3,4,5-tetrahydro-1H-benzo[d]azepin-7-yl}-acrylamide, (E)-3-{3-[2-(2-tert-Butyl-1H-pyrrolo[2,3-b]pyridin-3-yl)-ethyl]-2,3,4,5-tetrahydro-1H-benzo[d]azepin-7-yl}-N-hydroxy-acrylamide, (E)-N-Hydroxy-3-{3-[2-(1,3,5-trimethyl-1H-pyrazol-4-yl)-ethyl]-2,3,4,5-tetrahydro-1H-benzo[d]azepin-7-yl}-acrylamide, (E)-N-Hydroxy-3-{3-[2-(1-methyl-3-phenyl-1H-pyrazol-4-yl)-ethyl]-2,3,4,5-tetrahydro-1H-benzo[d]azepin-7-yl}-acrylamide, (E) —N-Hydroxy-3-{3-[2-(3-phenyl-1H-pyrazol-4-yl)-ethyl]-2,3,4,5-tetrahydro-1H-benzo[d]azepin-7-yl}-acryl amide, (E)-N-Hydroxy-3-{3-[2-(2-phenyl-2H-pyrazol-3-yl)-ethyl]-2,3,4,5-tetrahydro-1H-benzo[d]azepin-7-yl}-acryl amide, (E)-N-Hydroxy-3-{3-[2-(3-methyl-1-phenyl-1H-pyrazol-4-yl)-ethyl]-2,3,4,5-tetrahydro-1H-benzo[d]azepin-7-yl}-acrylamide, (E)-N-Hydroxy-3-{3-[2-(3-methyl-5-phenyl-isoxazol-4-yl)-ethyl]-2,3,4,5tetrahydro-1H-benzo[d]azepin-7-yl}-acrylamide, (E)-N-Hydroxy-3-{2-[2-(2-methyl-1H-indol-3-yl)-ethyl]-2,3,4,5-tetrahydro-1H-benzo[c]azepin-7-yl}-acrylamide, (E)-3-{2-[2-(2-tert-Butyl-1H-indol-3-yl)-ethyl]-2,3,4,5-tetrahydro-1H-benzo[c]azepin-7-yl}-N-hydroxy-acrylamide, (E)-N-Hydroxy-3-[2-(2-pyrazolo[1,5-a]pyridin-3-yl-ethyl)-2,3,4,5-tetrahydro-1H-benzo[c]azepin-7-yl]-acrylamide, (E)-N-Hydroxy-3-{2-[2-(2-methyl-pyrazolo[1,5-a]pyridin-3-yl)-ethyl]-2,3,4,5-tetrahydro-1H-benzo[c]azepin-7-yl}-acrylamide, (E)-3-{2-[2-(2-Ethyl-pyrazolo[1,5-a]pyridin-3-yl)-ethyl]-2,3,4,5-tetrahydro-1H-benzo[c]azepin-7-yl}-N-hydroxy-acrylamide, (E)-N-Hydroxy-3-{2-[2-(2-methyl-1H-pyrrolo[2,3-b]pyridin-3-yl)-ethyl]-2,3,4,5-tetrahydro-1H-benzo[c]azepin-7-yl}-acrylamide, (E)-3-{2-[2-(2-tert-Butyl-1H-pyrrolo[2,3-b]pyridin-3-yl)-ethyl]-2,3,4,5-tetrahydro-1H-benzo[c]azepin-7-yl}-N-hydroxy-acrylamide, (E)-N-Hydroxy-3-{2-[2-(1,3,5-trimethyl-1H-pyrazol-4-yl)-ethyl]-2,3,4,5-tetrahydro-1H-benzo[c]azepin-7-yl}-acrylamide, (E)-N-Hydroxy-3-{2-[2-(1-methyl-3-phenyl-1H-pyrazol-4-yl)-ethyl]-2,3,4,5-tetrahydro-1H-benzo[c]azepin-7-yl}-acrylamide, (E) —N-Hydroxy-3-{2-[2-(3-phenyl-1H-pyrazol-4-yl)-ethyl]-2,3,4,5-tetrahydro-1H-benzo[c]azepin-7-yl}-acrylamide, (E)-N-Hydroxy-3-{2-[2-(2-phenyl-2H-pyrazol-3-yl)-ethyl]-2,3,4,5-tetrahydro-1H-benzo[c]azepin-7-yl}-acrylamide, (E)-N-Hydroxy-3-{2-[2-(3-methyl-1-phenyl-1H-pyrazol-4-yl)-ethyl]-2,3,4,5-tetrahydro-1H-benzo[c]azepin-7-yl}-acrylamide, (E)-N-Hydroxy-3-{2-[2-(3-methyl-5-phenyl-isoxazol-4-yl)-ethyl]-2,3,4,5-tetrahydro-1H-benzo[c]azepin-7-yl}-acrylamide.

19. The treatment of claim 18, wherein the deacetylase-associated disorder is selected from a group consisting of a proliferative disease, a hyperproliferative disease; a disease of the immune system, a disease of the central nervous system, a disease associated with misexpression of a gene, or peripheral nervous system.

20. The treatment of claim 19, wherein the deacetylase-associated disorder is an HDAC dependent disease, wherein the HDAC is selected from the group of HDAC1, HDAC2, HDAC3, HDAC4, HDAC5, HDAC6, HDAC7, HDAC8, HDAC9, HDAC10 and HDAC11, or any combinations thereof.

21. The treatment of claim 20, wherein the proliferative disease is selected from the group consisting of a hyperproliferative disease, a benign tumor, a malignant tumor, a carcinoma of the brain, kidney, liver, adrenal gland, bladder, breast, stomach, ovaries, esophagus, colon, rectum, prostate, pancreas, lung, vagina, or thyroid, a sarcoma, glioblastomas, multiple myeloma, gastrointestinal cancer, colon carcinoma, colorectal adenoma, a tumor of the neck or head, an epidermal hyperproliferation, psoriasis, prostate hyperplasia, a neoplasia, mammary carcinoma, and a leukemia.

22. The treatment of claim 21, wherein the deacetylase-associated disorder is triggered by persistent angiogenesis, such as psoriasis; Kaposi's sarcoma; restenosis, endometriosis; Crohn's disease; Hodgkin's disease; leukemia; arthritis, such as rheumatoid arthritis; hemangioma; angiofibroma; eye diseases, such as diabetic retinopathy and neovascular glaucoma; renal diseases, such as glomerulonephritis; diabetic nephropathy; malignant nephrosclerosis; thrombotic microangiopathic syndromes; transplant rejections and glomerulopathy; fibrotic diseases, such as cirrhosis of the liver; mesangial cell-proliferative diseases; arteriosclerosis; injuries of the nerve tissue; and for inhibiting the re-occlusion of vessels after balloon catheter treatment, for use in vascular prosthetics or after inserting mechanical devices for holding vessels open, as immunosuppressants, as an aid in scar-free wound healing, and for treating age spots and contact dermatitis.

23. The treatment according to claim 21, wherein the deacetylase-associated disorder is a disease of the immune system.

24. The treatment according to claim 23, wherein the hyperproliferative disease is selected from the group consisting of leukemias, hyperplasias, fibrosis (including pulmonary, but also other types of fibrosis, such as renal fibrosis), angiogenesis, psoriasis, atherosclerosis and smooth muscle proliferation in the blood vessels, such as stenosis or restenosis following angioplasty.

25. A compound of formula I: 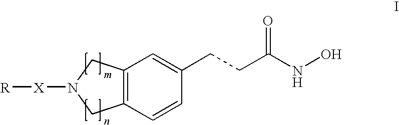
and pharmaceutically acceptable salts, wherein
the dashed line indicates a single or double bond,
n and m are each, independently, 1, 2, or 3, and the sum of n and m is 2, 3 or 4;
wherein X is (CH₂)ⱼ wherein each CH₂ may be independently replaced one or more times with C(O), S(O)₂, S(O), O, or NR², wherein R² is selected from the group consisting of H, alkyl, aryl, heterocycle, C₁₋₄-alkyl, and C₃₋₆-cycloalkyl;
j is an integer between 0 and 6.
R is selected from the group consisting of C₁₋₄-alkyl, C₃₋₆-cycloalkyl and aryl, wherein cycloalkyl and aryl may be further independently substituted one or more times with aryl, heterocycle, C₁₋₄-alkyl, C₁₋₄-alkoxy, halogen, amino, nitro, cyano, pyrrolidinyl or CF₃.
